SEQUENCE LISTING (120 sequences)

SEQ ID NO: 1 - DNA, 114 bp, Artificial sequence
  Regulator DNA Sequence
gttttaactcttgattactaatcctacctttcctctttatccacaaagtgtggataagttgtggattgatttcacacagcttgtgtagaaggttgtccacaagttgtgaaattt

SEQ ID NO: 2 - DNA, 72 bp, Artificial sequence
  Regulator DNA Sequence
ggataaatataaataatattcatatatgatttgcacttatcgccgctctcgtcctttgggcgggagcttttt

SEQ ID NO: 3 - DNA, 160 bp, Artificial sequence
  Regulator DNA Sequence
cgttgtaactttttataggtccattcatgacaaaggagggcggagtttccagaaaatccggtgaaaaccgtgagaaaagaactgatttttttggttatatgatgctaatccattcaaaaagatccatttctcaaagacaaaagactcccctttcctaaac

SEQ ID NO: 4 - DNA, 216 bp, Artificial sequence
  Regulator DNA Sequence
gcctgcctgttcagggcatggaattaagactcggaatcggagcttggcattgccttgcacagtggtgaacacaacaggtgacaatgcttgtcatcagtacggaaactgaaaagaacaaacagtcggagcaaacgctcatcatcggtttaaccaaacatttggaagcaacaaatgctataatacaaacagtgcctaatgttgggcactgtttatttt

SEQ ID NO: 5 - DNA, 93 bp, Artificial sequence
  Regulator DNA Sequence
atattatcggagtctgggagcgagttggcctgactccggcaaacggccttgccaaagagggcggagcgagcttcatatctgtcctcgtttttt

SEQ ID NO: 6 - DNA, 95 bp, Artificial sequence
  Regulator DNA Sequence
ggtttccaaatggttgtaataaaaatgatatggtgcgaaacataaatattccggcttgccgcggtgtatttattgttgtgtgatgccttgatttt

SEQ ID NO: 7 - DNA, 130 bp, Artificial sequence
  Regulator DNA Sequence
acaatttataatcaaactttgacttttggaggtggtatgatggggccctctgcttaggcaacaatacaccgcgtacctcaatgttaaggaaaaatgattatatcaggaggtgactccttatcctttgttg

SEQ ID NO: 8 - DNA, 98 bp, Artificial sequence
  Regulator DNA Sequence
ggtgttaaatacacacgcttaacgatttatgcagagggtgctgcaggcggcagttctgtacaaaaatgacctaagcggaggaaaaaaaccattatatt

SEQ ID NO: 9 - DNA, 222 bp, Artificial sequence
  Regulator DNA Sequence
gaacagctttttttgaataccttgtccagctgggtttcggccgttcgatttgcgaaggtctgctgctttatatataataaacaaacatacgttctcatagtgtaaaaacagcactagccgatgttttgagaagaactgattcgttagctctatgttgcgattgcaaacatattggttataatgaggagtccaaggcagtactggctcgaggcgaggtttata

SEQ ID NO: 10 - DNA, 93 bp, Artificial sequence
  Regulator DNA Sequence
tgaatttcaactggcgataaaggactatattagatggaagagaaatgatttgtcattgctacatactcatttatttagttctttgctccagtt

SEQ ID NO: 11 - DNA, 90 bp, Artificial sequence
  Regulator DNA Sequence
tggagatattcacaggcgaaatgtttacccgccttgaatatgagcacgatacccgcttcttcaggctcctaaagccagggcgggttttct

SEQ ID NO: 12 - DNA, 150 bp, Artificial sequence
  Regulator DNA Sequence
ataaaattttaccatatttaaggagggagttgaatttttttccttaggtaatgtgaattcattaaagcataaaatatccagttagtccaagacggaaagcctgcggacactgaacttacagtattacgctgtatgtttggtgtccgtttt

SEQ ID NO: 13 - DNA, 157 bp, Artificial sequence
  Regulator DNA Sequence
agtgttgatggatttgtaataggatcttgttatgagtctatcgttttataaaaatcaaggattggtctgttagtaagacttgctagtgaactattccgccatgtgtgtttacaccttcttagaattaagggcaggtgtatctatacatggctttttt

SEQ ID NO: 14 - DNA, 144 bp, Artificial sequence
  Regulator DNA Sequence
gtgtgaaatattgcagcctttttgttcagcttctatgttttcattttgttccttatcaataaggtgtatcgtgtaactctctgctgctggagcgaggatacatgaaaacaaaatgtgcatggtcgaatagagcagacggttttt

SEQ ID NO: 15 - DNA, 111 bp, Artificial sequence
  Regulator DNA Sequence
gattatatgctcattcattcacataattataataaaataatatcatttcccctgaaagggagaatattagaatgaaagctaatggtatgcaaaatctattagcttcttttg

SEQ ID NO: 16 - DNA, 82 bp, Artificial sequence
  Regulator DNA Sequence
taacgaatggtcttagagagcggatcaaaacgcgaatgatatacgttcagcaaagcttggtcgttcagcaccaggctttctt

SEQ ID NO: 17 - DNA, 131 bp, Artificial sequence
  Regulator DNA Sequence
cggggaaacccatacagcagaaagggagtttttttcatgattatccaattcattcgttatagataatgacaggtgtagacaaactgccagacctggccaatttttgagataggcctaggtttatttgtcta

SEQ ID NO: 18 - DNA, 89 bp, Artificial sequence
  Regulator DNA Sequence
agtaaaatttagagatgatataaaaacattcggtgcaaagcatgttgcagcgatgaacaaaagagcagtcgtttatgctgctcttttct

SEQ ID NO: 19 - DNA, 124 bp, Artificial sequence
  Regulator DNA Sequence
atatcccaaagtttaagccacctatagtttctactgcaaaacgtataatttagttcccacatatactaaaaaacgtgtccttaactctctctgtcagattagttgtaggtggcttaaacttagt

SEQ ID NO: 20 - DNA, 103 bp, Artificial sequence
  Regulator DNA Sequence
tataaacaaatatccttattcaggaatgttaccaacttgggcataacctagataaatggagaactggctttttgggctagttttcaatttatctgggttttta

SEQ ID NO: 21 - DNA, 185 bp, Artificial sequence
  Regulator DNA Sequence
atcccatagagattgtcatttgaaataagctaattgaaaataaattacttcccccatagaaatattcttaattgcttatttcaaataaacactcctttaagatagttgtagcacagaatttcccagattctgtgctataattatgtagaaatagagatgctctgaaaatgaggctctcttttttt

SEQ ID NO: 22 - DNA, 83 bp, Artificial sequence
  Regulator DNA Sequence
tagagcgctagttttttatacttaatcatgtgataattgtcacttagtaagcgtgaagccaaattgctttgcgtttttttcgc

SEQ ID NO: 23 - DNA, 207 bp, Artificial sequence
  Regulator DNA Sequence
atcaaatgaaaaggagtgttatcttaagccaatgaaattctaatcctgtattatatgaaaaaatgaagaaagctatccacttctcggatagaaactattttttcggacgggattcgtatgttcatttcgcatgataactcttgctataggcctttttggcgtagccccgcggattcctctctgaaaatattcaggaggttatttttt

SEQ ID NO: 24 - DNA, 122 bp, Artificial sequence
  Regulator DNA Sequence
tgtgttgaaatgaatagcaattttttatggtgagacaattaatgacaaagatgtcagttaatgctttttttataaaaagactccagagtgcctatttagaggtgttcttgggtctttttttg

SEQ ID NO: 25 - DNA, 93 bp, Artificial sequence
  Regulator DNA Sequence
tggaaaacaatattgcgcagtctgtccagttataattcggatagactggcattttgtcttcttgtgggaaaatgcggttgatttgtgtttcat

SEQ ID NO: 26 - DNA, 125 bp, Artificial sequence
  Regulator DNA Sequence
ttgagtaaaatgaatgagtgtgttgtttacccccagatttaattctgtggtatatcgcgtaaccggcggctgctatggtgatggtatatgaaggtaaactgccctgatttggttttacgtcacgt

SEQ ID NO: 27 - DNA, 214 bp, Artificial sequence
  Regulator DNA Sequence
cgttgcataataaaacaaaggaggaatggacgatgaataattatatgaagacttcaaacaaggcattattgcgcttaaaaatagaatttatttcaccggaccatttcgcgagcaagggaactctcctttaatttggttttattttatacaaagtggtgacaaataaatatattttttcgggtaataatgggcagaaataggtccattattttta

SEQ ID NO: 28 - DNA, 102 bp, Artificial sequence
  Regulator DNA Sequence
cgtgaaataaatcacaaacaaataccccctttatttgattgtgtgaatttctccttttgattgcgctgtaagaagccgtggcattttgctacggctttttac

SEQ ID NO: 29 - DNA, 121 bp, Artificial sequence
  Regulator DNA Sequence
caataactaaaaatatacattggctatttcagcatgattatagaagacaagtcgcatgatgtggatattttccaccattttttctttatgaaaataaggataagatgatgcgttttttcta

SEQ ID NO: 30 - DNA, 240 bp, Artificial sequence
  Regulator DNA Sequence
attaatgaaaagaggtgagatgacatgtttcttttgaatgcgcgaactagtatagctaaatttttgtatattgtccgtgcaattttttccggtattgcagttacattggcgaagtctgcccaaaaaactgtataacgagaaaaaatgagagaagcatggagtcacgcaatcggtttgtatagtgctaaattagtattatacttataattggcgtcgggcgcttgtctgacgcttttttgt

SEQ ID NO: 31 - DNA, 97 bp, Artificial sequence
  Regulator DNA Sequence
gtggaatgaaatttttgttactcattgtactttagttcatcggaatgctcgctcattctttcacttgttgagcaataaaaattttattcaggagtac

SEQ ID NO: 32 - DNA, 176 bp, Artificial sequence
  Regulator DNA Sequence
tagctagacaaagaagagtacggaacaaaagtgaaccactttttaaccgcctgattttcggctgtttaaaagtagcccctgcggaattaagccaaaattttctaaaaatcgtaaaacaatttttgaaaatcctttcttaattcttggtgctgaccactttttaaccgccttttctt

SEQ ID NO: 33 - DNA, 98 bp, Artificial sequence
  Regulator DNA Sequence
atcaataaatagacagattatttaatcgctgggttgaataattgaaggattctgatttttgcgtaagccttgaagcaaaaagaatgagtccttttttt

SEQ ID NO: 34 - DNA, 111 bp, Artificial sequence
  Regulator DNA Sequence
agtaaagtaaataaaggtttctcaaaaacagcgtaatgacctgggagagaacaacgataacaatgaaccattcccatgagtgttgcctggcaatatttgtggggatttttt

SEQ ID NO: 35 - DNA, 107 bp, Artificial sequence
  Regulator DNA Sequence
attgccggaagatgacgagaaaaattgaatcgtagtaaataaaatagatctatttctatgtttaataataaatgtgaagctgaacgaccacgtttggcttttctaat

SEQ ID NO: 36 - DNA, 110 bp, Artificial sequence
  Regulator DNA Sequence
aggttaagcaatctctttttttggaacaagagaaacttttggaatcatttcctataaatcatgaagagattatgacaaacgttcttctaatccaagaacgtcttttgtcc

SEQ ID NO: 37 - DNA, 76 bp, Artificial sequence
  Regulator DNA Sequence
gggcacctttaagaaggtcagaagtttgaacgaacttacaagctccctattcgtaatggatagggagcttttctat

SEQ ID NO: 38 - DNA, 171 bp, Artificial sequence
  Regulator DNA Sequence
aattaaacgaggaggtataattgtatgttggcatctatcagaatttggattgactttttgacaaaataaattgaataaatcttatcaaaaagtctgaaattaaaattcatgggtggcgtgtgtacttttatacgaaactacaaggatagcgattaggctgtccttttttgc

SEQ ID NO: 39 - DNA, 143 bp, Artificial sequence
  Regulator DNA Sequence
gtattgaataaagaatggttgggcgaaagcttcaaggattcagttcttcaaggggtgagaagaacaatttttgagtttgcgcctttttgcatacctaaaacaaaagggctgagcgtatctctatttcgctagtcctttttctt

SEQ ID NO: 40 - DNA, 86 bp, Artificial sequence
  Regulator DNA Sequence
tgaaagctgaaataaaaatgttcagtcgttgatctgaagaattaagcgtttgggacataagtcattgcggcttatgtcccaaacgc

SEQ ID NO: 41 - DNA, 144 bp, Artificial sequence
  Regulator DNA Sequence
taataaaagacgaaaagagggattcgtgcgttgaaaatttaagttaattttttagagaaactgtataactttgcctgaatataacgggtggagtgtgcggattttcggaaattagcttaaagaattcaaccacgggtctttttg

SEQ ID NO: 42 - DNA, 233 bp, Artificial sequence
  Regulator DNA Sequence
cataaatgattgatgagaaagacaactgaacttgcccgcgatctattagagaggaaaatcttggctgaaaattttccagattggcacaagggatgaccactttcgaaacctttgttgaaacaaaggcggtcgcaccgttaacacgctagaggaacacagattatttgtgttcaaaattaggtggaaccacgatattttcgtcctagtattcgttagaatactaggactttttg

SEQ ID NO: 43 - DNA, 118 bp, Artificial sequence
  Regulator DNA Sequence
gaaaagtacctttaagatagtccgagaggctaacaaggcaaagttctgtaaaattctgtatttttgtatagtgaatgatacgagatgtcttgccctttttttgtgtgtaaggcttttt

SEQ ID NO: 44 - DNA, 98 bp, Artificial sequence
  Regulator DNA Sequence
caaatgaaacatgtctatacaagttcacaaggaggcgccaaacatgaccattttagaattaacattcgcaacgaaccaactaacgacgacttttttta

SEQ ID NO: 45 - RNA, 114 nt, Artificial sequence
  Regulator RNA sequence
guuuuaacucuugauuacuaauccuaccuuuccucuuuauccacaaaguguggauaaguuguggauugauuucacacagcuuguguagaagguuguccacaaguugugaaauuu

SEQ ID NO: 46 - RNA, 72 nt, Artificial sequence
  Regulator RNA sequence
ggauaaauauaaauaauauucauauaugauuugcacuuaucgccgcucucguccuuugggcgggagcuuuuu

SEQ ID NO: 47 - RNA, 160 nt, Artificial sequence
  Regulator RNA sequence
cguuguaacuuuuuauagguccauucaugacaaaggagggcggaguuuccagaaaauccggugaaaaccgugagaaaagaacugauuuuuuugguuauaugaugcuaauccauucaaaaagauccauuucucaaagacaaaagacuccccuuuccuaaac

SEQ ID NO: 48 - RNA, 216 nt, Artificial sequence
  Regulator RNA sequence
gccugccuguucagggcauggaauuaagacucggaaucggagcuuggcauugccuugcacaguggugaacacaacaggugacaaugcuugucaucaguacggaaacugaaaagaacaaacagucggagcaaacgcucaucaucgguuuaaccaaacauuuggaagcaacaaaugcuauaauacaaacagugccuaauguugggcacuguuuauuuu

SEQ ID NO: 49 - RNA, 93 nt, Artificial sequence
  Regulator RNA sequence
auauuaucggagucugggagcgaguuggccugacuccggcaaacggccuugccaaagagggcggagcgagcuucauaucuguccucguuuuuu

SEQ ID NO: 50 - RNA, 95 nt, Artificial sequence
  Regulator RNA sequence
gguuuccaaaugguuguaauaaaaaugauauggugcgaaacauaaauauuccggcuugccgcgguguauuuauuguugugugaugccuugauuuu

SEQ ID NO: 51 - RNA, 130 nt, Artificial sequence
  Regulator RNA sequence
acaauuuauaaucaaacuuugacuuuuggaggugguaugauggggcccucugcuuaggcaacaauacaccgcguaccucaauguuaaggaaaaaugauuauaucaggaggugacuccuuauccuuuguug

SEQ ID NO: 52 - RNA, 98 nt, Artificial sequence
  Regulator RNA sequence
gguguuaaauacacacgcuuaacgauuuaugcagagggugcugcaggcggcaguucuguacaaaaaugaccuaagcggaggaaaaaaaccauuauauu

SEQ ID NO: 53 - RNA, 222 nt, Artificial sequence
  Regulator RNA sequence
gaacagcuuuuuuugaauaccuuguccagcuggguuucggccguucgauuugcgaaggucugcugcuuuauauauaauaaacaaacauacguucucauaguguaaaaacagcacuagccgauguuuugagaagaacugauucguuagcucuauguugcgauugcaaacauauugguuauaaugaggaguccaaggcaguacuggcucgaggcgagguuuaua

SEQ ID NO: 54 - RNA, 93 nt, Artificial sequence
  Regulator RNA sequence
ugaauuucaacuggcgauaaaggacuauauuagauggaagagaaaugauuugucauugcuacauacucauuuauuuaguucuuugcuccaguu

SEQ ID NO: 55 - RNA, 90 nt, Artificial sequence
  Regulator RNA sequence
uggagauauucacaggcgaaauguuuacccgccuugaauaugagcacgauacccgcuucuucaggcuccuaaagccagggcggguuuucu

SEQ ID NO: 56 - RNA, 150 nt, Artificial sequence
  Regulator RNA sequence
auaaaauuuuaccauauuuaaggagggaguugaauuuuuuuccuuagguaaugugaauucauuaaagcauaaaauauccaguuaguccaagacggaaagccugcggacacugaacuuacaguauuacgcuguauguuugguguccguuuu

SEQ ID NO: 57 - RNA, 157 nt, Artificial sequence
  Regulator RNA sequence
aguguugauggauuuguaauaggaucuuguuaugagucuaucguuuuauaaaaaucaaggauuggucuguuaguaagacuugcuagugaacuauuccgccauguguguuuacaccuucuuagaauuaagggcagguguaucuauacauggcuuuuuu

SEQ ID NO: 58 - RNA, 144 nt, Artificial sequence
  Regulator RNA sequence
gugugaaauauugcagccuuuuuguucagcuucuauguuuucauuuuguuccuuaucaauaagguguaucguguaacucucugcugcuggagcgaggauacaugaaaacaaaaugugcauggucgaauagagcagacgguuuuu

SEQ ID NO: 59 - RNA, 111 nt, Artificial sequence
  Regulator RNA sequence
gauuauaugcucauucauucacauaauuauaauaaaauaauaucauuuccccugaaagggagaauauuagaaugaaagcuaaugguaugcaaaaucuauuagcuucuuuug

SEQ ID NO: 60 - RNA, 82 nt, Artificial sequence
  Regulator RNA sequence
uaacgaauggucuuagagagcggaucaaaacgcgaaugauauacguucagcaaagcuuggucguucagcaccaggcuuucuu

SEQ ID NO: 61 - RNA, 131 nt, Artificial sequence
  Regulator RNA sequence
cggggaaacccauacagcagaaagggaguuuuuuucaugauuauccaauucauucguuauagauaaugacagguguagacaaacugccagaccuggccaauuuuugagauaggccuagguuuauuugucua

SEQ ID NO: 62 - RNA, 89 nt, Artificial sequence
  Regulator RNA sequence
aguaaaauuuagagaugauauaaaaacauucggugcaaagcauguugcagcgaugaacaaaagagcagucguuuaugcugcucuuuucu

SEQ ID NO: 63 - RNA, 124 nt, Artificial sequence
  Regulator RNA sequence
auaucccaaaguuuaagccaccuauaguuucuacugcaaaacguauaauuuaguucccacauauacuaaaaaacguguccuuaacucucucugucagauuaguuguagguggcuuaaacuuagu

SEQ ID NO: 64 - RNA, 103 nt, Artificial sequence
  Regulator RNA sequence
uauaaacaaauauccuuauucaggaauguuaccaacuugggcauaaccuagauaaauggagaacuggcuuuuugggcuaguuuucaauuuaucuggguuuuua

SEQ ID NO: 65 - RNA, 185 nt, Artificial sequence
  Regulator RNA sequence
aucccauagagauugucauuugaaauaagcuaauugaaaauaaauuacuucccccauagaaauauucuuaauugcuuauuucaaauaaacacuccuuuaagauaguuguagcacagaauuucccagauucugugcuauaauuauguagaaauagagaugcucugaaaaugaggcucucuuuuuuu

SEQ ID NO: 66 - RNA, 83 nt, Artificial sequence
  Regulator RNA sequence
uagagcgcuaguuuuuuauacuuaaucaugugauaauugucacuuaguaagcgugaagccaaauugcuuugcguuuuuuucgc

SEQ ID NO: 67 - RNA, 207 nt, Artificial sequence
  Regulator RNA sequence
aucaaaugaaaaggaguguuaucuuaagccaaugaaauucuaauccuguauuauaugaaaaaaugaagaaagcuauccacuucucggauagaaacuauuuuuucggacgggauucguauguucauuucgcaugauaacucuugcuauaggccuuuuuggcguagccccgcggauuccucucugaaaauauucaggagguuauuuuuu

SEQ ID NO: 68 - RNA, 122 nt, Artificial sequence
  Regulator RNA sequence
uguguugaaaugaauagcaauuuuuuauggugagacaauuaaugacaaagaugucaguuaaugcuuuuuuuauaaaaagacuccagagugccuauuuagagguguucuugggucuuuuuuug

SEQ ID NO: 69 - RNA, 93 nt, Artificial sequence
  Regulator RNA sequence
uggaaaacaauauugcgcagucuguccaguuauaauucggauagacuggcauuuugucuucuugugggaaaaugcgguugauuuguguuucau

SEQ ID NO: 70 - RNA, 125 nt, Artificial sequence
  Regulator RNA sequence
uugaguaaaaugaaugaguguguuguuuacccccagauuuaauucugugguauaucgcguaaccggcggcugcuauggugaugguauaugaagguaaacugcccugauuugguuuuacgucacgu

SEQ ID NO: 71 - RNA, 214 nt, Artificial sequence
  Regulator RNA sequence
cguugcauaauaaaacaaaggaggaauggacgaugaauaauuauaugaagacuucaaacaaggcauuauugcgcuuaaaaauagaauuuauuucaccggaccauuucgcgagcaagggaacucuccuuuaauuugguuuuauuuuauacaaaguggugacaaauaaauauauuuuuucggguaauaaugggcagaaauagguccauuauuuuua

SEQ ID NO: 72 - RNA, 102 nt, Artificial sequence
  Regulator RNA sequence
cgugaaauaaaucacaaacaaauacccccuuuauuugauugugugaauuucuccuuuugauugcgcuguaagaagccguggcauuuugcuacggcuuuuuac

SEQ ID NO: 73 - RNA, 121 nt, Artificial sequence
  Regulator RNA sequence
caauaacuaaaaauauacauuggcuauuucagcaugauuauagaagacaagucgcaugauguggauauuuuccaccauuuuuucuuuaugaaaauaaggauaagaugaugcguuuuuucua

SEQ ID NO: 74 - RNA, 240 nt, Artificial sequence
  Regulator RNA sequence
auuaaugaaaagaggugagaugacauguuucuuuugaaugcgcgaacuaguauagcuaaauuuuuguauauuguccgugcaauuuuuuccgguauugcaguuacauuggcgaagucugcccaaaaaacuguauaacgagaaaaaaugagagaagcauggagucacgcaaucgguuuguauagugcuaaauuaguauuauacuuauaauuggcgucgggcgcuugucugacgcuuuuuugu

SEQ ID NO: 75 - RNA, 97 nt, Artificial sequence
  Regulator RNA sequence
guggaaugaaauuuuuguuacucauuguacuuuaguucaucggaaugcucgcucauucuuucacuuguugagcaauaaaaauuuuauucaggaguac

SEQ ID NO: 76 - RNA, 176 nt, Artificial sequence
  Regulator RNA sequence
uagcuagacaaagaagaguacggaacaaaagugaaccacuuuuuaaccgccugauuuucggcuguuuaaaaguagccccugcggaauuaagccaaaauuuucuaaaaaucguaaaacaauuuuugaaaauccuuucuuaauucuuggugcugaccacuuuuuaaccgccuuuucuu

SEQ ID NO: 77 - RNA, 98 nt, Artificial sequence
  Regulator RNA sequence
aucaauaaauagacagauuauuuaaucgcuggguugaauaauugaaggauucugauuuuugcguaagccuugaagcaaaaagaaugaguccuuuuuuu

SEQ ID NO: 78 - RNA, 111 nt, Artificial sequence
  Regulator RNA sequence
aguaaaguaaauaaagguuucucaaaaacagcguaaugaccugggagagaacaacgauaacaaugaaccauucccaugaguguugccuggcaauauuuguggggauuuuuu

SEQ ID NO: 79 - RNA, 107 nt, Artificial sequence
  Regulator RNA sequence
auugccggaagaugacgagaaaaauugaaucguaguaaauaaaauagaucuauuucuauguuuaauaauaaaugugaagcugaacgaccacguuuggcuuuucuaau

SEQ ID NO: 80 - RNA, 110 nt, Artificial sequence
  Regulator RNA sequence
agguuaagcaaucucuuuuuuuggaacaagagaaacuuuuggaaucauuuccuauaaaucaugaagagauuaugacaaacguucuucuaauccaagaacgucuuuugucc

SEQ ID NO: 81 - RNA, 76 nt, Artificial sequence
  Regulator RNA sequence
gggcaccuuuaagaaggucagaaguuugaacgaacuuacaagcucccuauucguaauggauagggagcuuuucuau

SEQ ID NO: 82 - RNA, 171 nt, Artificial sequence
  Regulator RNA sequence
aauuaaacgaggagguauaauuguauguuggcaucuaucagaauuuggauugacuuuuugacaaaauaaauugaauaaaucuuaucaaaaagucugaaauuaaaauucauggguggcguguguacuuuuauacgaaacuacaaggauagcgauuaggcuguccuuuuuugc

SEQ ID NO: 83 - RNA, 143 nt, Artificial sequence
  Regulator RNA sequence
guauugaauaaagaaugguugggcgaaagcuucaaggauucaguucuucaaggggugagaagaacaauuuuugaguuugcgccuuuuugcauaccuaaaacaaaagggcugagcguaucucuauuucgcuaguccuuuuucuu

SEQ ID NO: 84 - RNA, 86 nt, Artificial sequence
  Regulator RNA sequence
ugaaagcugaaauaaaaauguucagucguugaucugaagaauuaagcguuugggacauaagucauugcggcuuaugucccaaacgc

SEQ ID NO: 85 - RNA, 144 nt, Artificial sequence
  Regulator RNA sequence
uaauaaaagacgaaaagagggauucgugcguugaaaauuuaaguuaauuuuuuagagaaacuguauaacuuugccugaauauaacggguggagugugcggauuuucggaaauuagcuuaaagaauucaaccacgggucuuuuug

SEQ ID NO: 86 - RNA, 233 nt, Artificial sequence
  Regulator RNA sequence
cauaaaugauugaugagaaagacaacugaacuugcccgcgaucuauuagagaggaaaaucuuggcugaaaauuuuccagauuggcacaagggaugaccacuuucgaaaccuuuguugaaacaaaggcggucgcaccguuaacacgcuagaggaacacagauuauuuguguucaaaauuagguggaaccacgauauuuucguccuaguauucguuagaauacuaggacuuuuug

SEQ ID NO: 87 - RNA, 118 nt, Artificial sequence
  Regulator RNA sequence
gaaaaguaccuuuaagauaguccgagaggcuaacaaggcaaaguucuguaaaauucuguauuuuuguauagugaaugauacgagaugucuugcccuuuuuuuguguguaaggcuuuuu

SEQ ID NO: 88 - RNA, 98 nt, Artificial sequence
  Regulator RNA sequence
caaaugaaacaugucuauacaaguucacaaggaggcgccaaacaugaccauuuuagaauuaacauucgcaacgaaccaacuaacgacgacuuuuuuua

SEQ ID NO: 89 - DNA, 217 bp, Artificial sequence
  Regulator DNA Sequence
gtaagaaaaaatgaaagaaaggagtatgtgtcatgatcttagtaactaaaaccaaacaagccaaacaacacccactccttttagacgtcatgctatagacaagtgccatactaggccttatagccaagaatatagaagatgataagacgctaagctggagtggcaaggattaggactagattctgtgccttgtactgtccatcttagggtctttttt

SEQ ID NO: 90 - DNA, 38 bp, Artificial sequence
  single stranded  DNA oligonucleotide adaptor
  misc_feature (1)..(18): n is a, c, g, or t
  misc_feature (1)..(1): 5′ phosphorylated
  misc_feature (3)..(14): 12mer_index
  misc_feature (38)..(38): 3′ amino blocked
nnnnnnnnnnnnnnnnnnagatcggaagagcgtcgtgt

SEQ ID NO: 91 - DNA, 30 bp, Artificial sequence
  single stranded  DNA oligonucleotide adaptor
tctacactctttccctacacgacgctcttc

SEQ ID NO: 92 - DNA, 36 bp, Artificial sequence
  single stranded  DNA oligonucleotide adaptor
  misc_feature (1)..(1): 5′ phosphorylated
  misc_feature (2)..(2): n is a, c, g, or t
  misc_feature (36)..(36): 3′ amino blocked
nnagatcggaagagcacacgtctgaactccagtcac

SEQ ID NO: 93 - DNA, 50 bp, Artificial sequence
  single stranded  DNA oligonucleotide adaptor
aatgatacggcgaccaccgagatctacactctttccctacacgacgctct

SEQ ID NO: 94 - DNA, 49 bp, Artificial sequence
  single stranded  DNA oligonucleotide adaptor
  misc_feature (25)..(32): n is a, c, g, or t
  misc_feature (25)..(32): 8mer_index
caagcagaagacggcatacgagatnnnnnnnngtgactggagttcagac

SEQ ID NO: 95 - DNA, 22 bp, Artificial sequence
  single stranded  DNA oligonucleotide adaptor
gtcaatacgactcactataggg

SEQ ID NO: 96 - DNA, 19 bp, Artificial sequence
  single stranded  DNA oligonucleotide adaptor
caaaagctggtaccgggcc

SEQ ID NO: 97 - DNA, 36 bp, Artificial sequence
  single stranded  DNA oligonucleotide adaptor
atcaacccgggatcattttaacgacaaaccgagatg

SEQ ID NO: 98 - DNA, 50 bp, Artificial sequence
  single stranded  DNA oligonucleotide adaptor
ttttatttagcttgaataaaaagacaacagccgtgtcgtttgaaatacac

SEQ ID NO: 99 - DNA, 50 bp, Artificial sequence
  single stranded  DNA oligonucleotide adaptor
gtgtatttcaaacgacacggctgttgtctttttattcaagctaaataaaa

SEQ ID NO: 100 - DNA, 32 bp, Artificial sequence
  single stranded  DNA oligonucleotide adaptor
atcaaggatccccgccagcaagcgctatattt

SEQ ID NO: 101 - DNA, 230 bp, Artificial sequence
  Regulator DNA Sequence
aactatataaaaccacttaataaatatcgccgaacctattgttaaataaagtctgaatttcatactcccaagaaataattacatctgggtttttaccaccccttacttttttctcgggactttaacaatagttttatgatctcattatcaactatagacaaaatatgtctatcgttatcattctcgatttatttgggactcataacactatgtctgagtcccatttttta

SEQ ID NO: 102 - DNA, 254 bp, Artificial sequence
  Regulator DNA Sequence
attccagaaaaggagtaaaaaatatgcggcaaggtattcttaaataaaactataatcaaatagtgggaacaaagaattatgatagctccttttgtgggggcttggttttttgtacccaatttaagaatacttttgccttatcaattttgacatattcaaaaaacagcaatcacaaataggtgtatgctgtatatgtgtatgtccgcaaattagaatccccagtggtaaaagtattttactgctggggattttta

SEQ ID NO: 103 - DNA, 107 bp, Artificial sequence
  Regulator DNA Sequence
gttacaagcagaagccaccgcttctcacctgcgggcacacagcgcgttgggtcgaatgaaagatgacagagatttttcaagggtggctttgtgccgcccttttctat

SEQ ID NO: 104 - DNA, 169 bp, Artificial sequence
  Regulator DNA Sequence
aattaaaactacaaaccacaatctatgatcaatcatctaaaagaaaagactagtgacctatatgatagcagacaagtggtttgtacgagacgagtctaatttatttttcatcaaaaaataaataaaagaactaacgtgaccagacctttattgtctgttcccgtttttt

SEQ ID NO: 105 - DNA, 89 bp, Artificial sequence
  Regulator DNA Sequence
gtaacctcattgatttacctcaaacctgttgtgagttaagttaatgaagttgtaaccacgctgtttgctgagcttgactccgggcagtg

SEQ ID NO: 106 - DNA, 143 bp, Artificial sequence
  Regulator DNA Sequence
aacaaagagaaaggagaaaaccatcatgaacaaagtagatgttttgatgacacaattgaatacacagcttctccaatctcagttgagttctatgtaaaaattgaatgaacccaagatggaggagtctgtcttgggttttttgt

SEQ ID NO: 107 - DNA, 271 bp, Artificial sequence
  Regulator DNA Sequence
agaatgatttcggaggtacacggagacatgaaaaattaagtctattttttaaaggaaactttatttagtagattttgtcatgtttctgcatatccgcgggactttcgtttttttgtgctttcaagctcctatagcttatatttagattgatcctgtcactcctatgacagtgcaaattgtaagtctacaaacgaatgcccctatttagacactcctgtgtcttctttgtgcaatctcaacagctctgctaaataaagcagggctgtatttt

SEQ ID NO: 108 - DNA, 164 bp, Artificial sequence
  Regulator DNA Sequence
aataacatttcaaaaggagtcagtcaaacatgaagtcaactcaattttcagactttgctttgttgttgcgttactcgggctagtgcgaaagcattagtcctgtttggcttaccaagcgggagtaaatcaacatctcgcttggcttccagcgagatgtttttatt

SEQ ID NO: 109 - DNA, 157 bp, Artificial sequence
  Regulator DNA Sequence
tattttttatgccttgacattcctcatctctaacgatataatgtttacaatttaacctttaatcaagtccagtgaggctgcaaggtaggaaaaacggtataaggggcaatttgactgcctcgttttttgcaaaaccttgctgtggcgaggttttttc

SEQ ID NO: 110 - DNA, 272 bp, Artificial sequence
  Regulator DNA Sequence
agaactatttcggaggtacacggagacatgaaaaattaagtctattttttaaaggaaactttatttagtagattttgtcatgtttctgcatatccgcgggatgttcattttttgtgctttctagcttctatttgcttatctttagatgaatcctgtcgctcctatgacagtgtaagctggaagcctatagacgaatattcctacttagacactcctgtgtctgctttgtgcaactaaaaacagctctgctaaataaagcagggctgtatttt

SEQ ID NO: 111 - DNA, 315 bp, Artificial sequence
  Regulator DNA Sequence
tttaaaaaattctgaaatattcttgacaggtgatgaaaaaggtggtaagataggaaacatcaaaaaagaaagcagaaaaagaaatgaataagagacaagctgtaactatgaatcaaaacttttactttagctactttagatagtgtttgtagacatgtcaccatggatgcaaacagttcaagcaatttgtttgtatctatgtggctaagatttttgattgtggtccatatagatgaatatctaaatggactagctaagttgtaacttgttagattatttcaagcatccgtttagatagtatctaggcggtttttt

SEQ ID NO: 112 - DNA, 270 bp, Artificial sequence
  Regulator DNA Sequence
actaatactcggaggtaagggagacatgaacaactaatctatcaaataaagaacctttatttagtagatcttgtttttgtctcttttggtgtgctctttttgtactagattttctagtactttatcatctatgaaaatcaaaaaactagaaagctatgctcctcttgggttgggttaggcaactccaagttagtttgacgagtttttcaacgaggataatagagttttgacagtgactttgggctctactagataaagtagagctttttg

SEQ ID NO: 113 - DNA, 258 bp, Artificial sequence
  Regulator DNA Sequence
caaataatcaggaggtagggtgcattgaaaaattaattttcttcttgtttgattggactttttgacgagactgattgagttggcttcgaccgactggattcagttaccttgtctcgttttgaagaaaatcgccaaagttgtgagactttatctagaagaaaattgattgcatgcacattatcataaattataagctagatactagtcttttttgtgcggtcaaacaagttctactggataaggtaggacttgtttttt

SEQ ID NO: 114 - DNA, 271 bp, Artificial sequence
  Regulator DNA Sequence
agtaatagtgccacctaacccacagatcaagacgtaactgtatgaaataaacaatgtttatttactacatgttctttttctgtgtttttctctcgtgtttttatagtacattttgtactatgttctggtgattcaaattgaaaaaagtacaaacgtattgtggtgttcccttcccttaccgaagtggaacgtactttcagcactttttgaagcaccataatacacttttcagactcagtttcccgtgtagtacctaaactacacgtttttc

SEQ ID NO: 115 - DNA, 271 bp, Artificial sequence
  Regulator DNA Sequence
aagaatatttcggaggtacacggagacatgaaaaattaagtctattttttaaaggaaactttatttagtagattttgtcatgtttctgcatatccgcaggatgttcgttttcgatgctttctaacttctatttgcttatctttagattaatcctgtcgctcctatgacagtgtaagctggaagcctatgaacgattaatcctacttgaacactcctatgtctgatttgtgcaatttcaacagctctgctaaataaagcagggctgtatttt

SEQ ID NO: 116 - DNA, 247 bp, Artificial sequence
  Regulator DNA Sequence
aagaagaaacacaaaccacaaaaggatgatcaagaaactacatcttttcatcagagaattaaatagaggagttgtggaatgtgacttcacttggatctaaagaatcaatcaaagggaacatccaccactgtctgttcccttttttctatcgatttttcggtcggttaagtcaattcctcataccgatgacaaccgggaactctccagtcacgagctagtgacgtggtagttcccttttttgtctcgt

SEQ ID NO: 117 - DNA, 179 bp, Artificial sequence
  Regulator DNA Sequence
caggctgtcccgcaaacgggccaagcgaacgccacaaatgggaggtccatagggcggtgtgcagacacaccatcggcgggatgaccccgcggcgcgaccccatgccctagcagcctgaagagggctctctcttcaggtatccaacggtggctcgcgctgcgctaagccaccgttttgct

SEQ ID NO: 118 - DNA, 202 bp, Artificial sequence
  Regulator DNA Sequence
aaaagaagactaatagctatatgcaacaagtccagtctatgatgattatgcgaatgtatgctgacacgatgatgtggctcagcatgatgacgcatatccataggcgaagtagcttgttgtatacctgttagtatatagcatacccatacaaagagtttgtaagaagcgcttctcctggatttcctcgagaagcgcttcttcg

SEQ ID NO: 119 - DNA, 132 bp, Artificial sequence
  Regulator DNA Sequence
atcaaatatatgaacttagatattaggatacaattaggtttcgagaacctaagcgttgctccgctattacgttcatctacgattagtgtatgatagaagaaaacgggccacgcagacgttgcccgtttttct

SEQ ID NO: 120 - DNA, 181 bp, Artificial sequence
  Regulator DNA Sequence
gattttaagaattatcgtttaactaaatacaaaaatacgcacgaagaaatgaaatatatcaagttacatatcgcatctgtattacctttccacaaggtaacgcttcgtggcgtatccgcttgattagaacaatagtataatatatagaagagcccgaagcaccccgcttcgggctttttgt

(19) United States
(12) Patent Application Publication
(10) Pub. No.: US 2018/0237774 A1
(43) Pub. Date: Aug. 23, 2018

(54) METHODS OF SCREENING FOR RIBOSWITCHES AND ATTENUATORS

(71) Applicant: Yeda Research and Development Co. Ltd., Rehovot (IL)

(72) Inventors: Rotem SOREK, Rehovot (IL); Daniel DAR, Rehovot (IL)

(21) Appl. No.: 15/750,193

(22) PCT Filed: Aug. 2, 2016
(86) PCT No.: PCT/IL2016/050843

§ 371 Date: Feb. 4, 2018

Related U.S. Application Data
(60) Provisional application No. 62/200,662, filed on Aug. 4, 2015.
(60) Provisional application No. 62/289,261, filed on Jan. 31, 2016.

(51) Int. Cl. C12N 15/113; C12N 15/115; C12N 15/67; C07H 21/02; C12Q 1/04; G01N 33/58

(52) U.S. Cl. CPC C12N 15/113; C12N 15/115; G01N 33/582; C07H 21/02; C12Q 1/04; C12N 15/67

(57) ABSTRACT

A method of determining a transcription termination site in bacterial DNA is disclosed. Uses of sequences comprising transcription termination sites are also disclosed.

### FIELD AND BACKGROUND OF THE INVENTION

The present invention, in some embodiments thereof, relates to isolated polynucleotides that serve as bacterial transcription terminators, methods of identifying same and uses thereof.

Riboswitches and attenuators are 5′UTR-residing, cis-regulatory RNA elements that tune gene expression in bacteria by sensing key metabolites, amino acids, nucleotides and ions. These RNA elements can regulate the expression of the downstream gene either at the transcription or the translation level. When riboswitches and attenuators control transcription they usually generate a condition-specific, regulated transcriptional terminator, such that termination results in a prematurely aborted transcript whereas read-through generates a full length, productive mRNA (FIG. 1A). In the case of riboswitches, the 5′UTR RNA sensor differentially folds to form a terminator or an antiterminator in the presence or absence of the regulating metabolite, respectively; in attenuators, the formation of a transcriptional terminator is mediated by the rate of translation of an upstream ORF (uORF), as exemplified in the classic case of the Trp operon. Regulation by conditional termination is known to control key processes in bacteria including core metabolism, motility and biofilm formation, and virulence. Riboswitches enable optimization of metabolite production in bacterial expression systems, are readily applicable components for synthetic biology applications, and also form potential therapeutic targets for novel classes of antibiotics.

Significant efforts have been invested in the discovery of new riboswitches and attenuators that sense novel metabolites. However, only ˜25 classes of naturally occurring riboswitches have been described so far, although it is estimated that hundreds more exist in bacteria. To date, almost all known riboswitches have been discovered via comparative-genomics-based approaches in which intergenic regions are compared across multiple bacterial phyla to identify conserved sequences and structures. Once such a conserved 5′UTR is detected, its possible ligand is predicted based on the identity of the downstream gene, and extensive in-vitro verification experiments are then performed. This approach has been highly successful at identifying riboswitches that are conserved across a wide phylogenetic range. However, most of the yet to be discovered elements are predicted to be restricted to specific clades of bacteria, and for such elements current conservation-based approaches perform poorly. There is currently no experimental method that enables genome-wide discovery of riboswitches and other conditional termination regulators. Furthermore, given a metabolite of interest, there is no efficient approach that can identify natural riboswitches or attenuators that sense and respond to it.

Background art includes U.S. Pat. No. 8,440,810, Topp S, et al., ACS Chemical biology. 2010; 5(1):139-148 and Blount et al., Antimicrob. Agents Chemother. doi:10.1128/AAC.01282-15.

### SUMMARY OF THE INVENTION

According to an aspect of some embodiments of the present invention there is provided an isolated polynucleotide comprising a nucleic acid sequence as set forth in SEQ ID NOs: 1-44 operatively linked to a heterologous nucleic acid sequence.

According to an aspect of some embodiments of the present invention there is provided an isolated RNA comprising a nucleic acid sequence as set forth in SEQ ID NOs: 45-88, or a DNA encoding same, wherein the RNA or DNA is no longer than 450 nucleotides.

According to an aspect of some embodiments of the present invention there is provided an RNA aptamer comprising a nucleic acid sequence as set forth in SEQ ID NOs: 45-88 operatively linked to a signal generating moiety.

According to an aspect of some embodiments of the present invention there is provided a bacteria genetically modified to express the isolated polynucleotide described herein.

According to an aspect of some embodiments of the present invention there is provided a cell which comprises the aptamer described herein.

According to an aspect of some embodiments of the present invention there is provided a bacteria genetically modified to express an isolated polynucleotide comprising a nucleic acid sequence as set forth in SEQ ID NOs: 23, 27, 38 and 41 operatively linked to a reporter polypeptide.

According to an aspect of some embodiments of the present invention there is provided a method of detecting an antibiotic in a sample comprising:

(a) culturing a L. monocytogenes or E. faecalis bacteria in a medium comprising the sample;

(b) analyzing the number of full length RNA transcripts transcribed from the bacterial gene selected from the group consisting of lmo0919, lmo1652, EF1413 and EF2720 and prematurely terminated RNA transcripts transcribed from the bacterial gene; and

(c) comparing the ratio of prematurely terminated RNA transcripts transcribed from the bacterial gene: full length RNA transcripts transcribed from the bacterial gene in the presence of the sample to the ratio of prematurely terminated RNA transcripts transcribed from the bacterial gene: full length RNA transcripts transcribed from the bacterial gene in the absence of the sample, wherein a statistically significant change in the ratio is indicative that the sample comprises an antibiotic.

According to an aspect of some embodiments of the present invention there is provided a method of detecting an antibiotic in a sample comprising:

(a) culturing the bacteria of claim 16 in a medium comprising the sample; and

(b) measuring a level of expression of the reporter polypeptide, wherein a change in the level of expression of the reporter polypeptide as compared to the level of the reporter polypeptide measured when the bacteria of claim 16 are cultured in a medium devoid of an antibiotic, is indicative that the sample comprises an antibiotic.

According to an aspect of some embodiments of the present invention there is provided a method of detecting an antibiotic in a sample comprising:

(a) contacting the aptamer described herein with the sample; and

(b) measuring the signal generated by the signal generating moiety, wherein a level of the signal above a predetermined threshold is indicative that the sample comprises an antibiotic.

According to an aspect of some embodiments of the present invention there is provided a method of determining whether an agent is a transcription terminator comprising:

(a) culturing the bacteria described herein in a medium comprising the agent; and

(b) measuring the level of expression of the reporter polypeptide, wherein a change in said level of expression of said reporter polypeptide as compared to the level of said reporter polypeptide measured when the bacteria are cultured in a medium devoid of the agent is indicative that the agent is a transcription terminator.

According to an aspect of some embodiments of the present invention there is provided a method of identifying if an agent is an antibiotic, the method comprising:

determining whether the agent is a transcription terminator as described herein; and

testing an effect of the transcription terminator on vitality of bacterial cells, wherein a level of vitality of bacterial cells below a predetermined amount is indicative that the agent is an antibiotic.

According to an aspect of some embodiments of the present invention there is provided a method of controlling expression of a gene product comprising contacting a bacteria with a ligand of a ligand responsive element, wherein the bacteria comprises a nucleic acid sequence encoding the gene product, the nucleic acid sequence being operatively linked to:

(i) the ligand responsive element, wherein the ligand responsive element comprises a sequence as set forth in SEQ ID NOs: 1-44; and

(ii) a promoter, thereby controlling expression of the gene product, thereby controlling expression of the gene product.

According to an aspect of some embodiments of the present invention there is provided a method of determining a transcription termination site in bacterial DNA:

(a) ligating a first adaptor to the 3′ end of RNA transcripts of a bacterial RNA sample to generate elongated RNA transcripts;

(b) fragmenting the elongated RNA transcripts;

(c) combining the elongated RNA transcripts with a reverse transcriptase and an oligonucleotide that hybridizes to the adaptor under conditions that allow synthesis of cDNA from the elongated RNA transcripts;

(d) ligating a second adaptor to the 3′ end of the cDNA to generate elongated cDNA transcripts;

(e) amplifying the elongated cDNA transcripts using primers that hybridize to the sequence of the first adaptor and the sequence of the second adaptor to generate amplified DNA; and

(f) sequencing the amplified DNA, thereby determining the transcription termination site in bacterial DNA.

According to an aspect of some embodiments of the present invention there is provided a method of determining a transcription termination site in bacterial DNA:

(a) ligating a first adaptor to the 3′ end of RNA transcripts of a bacterial RNA sample to generate elongated RNA transcripts;

(b) fragmenting the elongated RNA transcripts to generate fragmented RNA transcripts;

(c) ligating a second adaptor to the 5′ end of the fragmented RNA transcripts to generate elongated fragmented RNA transcripts;

(d) combining the elongated fragmented RNA transcripts with a reverse transcriptase and an oligonucleotide that hybridizes to the first adaptor under conditions that allow synthesis of cDNA from the elongated fragmented RNA transcripts;

(e) amplifying the cDNA transcripts using primers that hybridize to the sequence of the first adaptor and the sequence of the second adaptor to generate amplified DNA; and

(f) sequencing the amplified DNA, thereby determining the transcription termination site in bacterial DNA.

According to an aspect of some embodiments of the present invention there is provided a method of determining whether a ligand can control premature transcription termination of a bacterial gene comprising:

(a) culturing bacteria in a medium comprising the ligand;

(b) analyzing the number of full length RNA transcripts transcribed from the bacterial gene and the number of prematurely terminated RNA transcripts transcribed from the bacterial gene; and

(c) comparing the ratio of prematurely terminated RNA transcripts transcribed from the bacterial gene: full length RNA transcripts transcribed from the bacterial gene in the presence of the ligand to the ratio of prematurely terminated RNA transcripts transcribed from the bacterial gene: full length RNA transcripts transcribed from the bacterial gene in the absence of the ligand, wherein a statistically significant change in the ratio is indicative that the ligand can control premature transcription termination of the bacterial gene.

According to some embodiments of the invention, the heterologous nucleic acid sequence encodes a polypeptide.

According to some embodiments of the invention, the polypeptide is a human polypeptide.

According to some embodiments of the invention, the polypeptide is a reporter polypeptide comprising a detectable moiety.

According to some embodiments of the invention, the detectable moiety is a fluorescent moiety or a phosphorescent moiety.

According to some embodiments of the invention, the isolated polynucleotide is operatively linked to a promoter.

According to some embodiments of the invention, the promoter is a bacterial promoter.

According to some embodiments of the invention, the nucleic acid sequence is as set forth in SEQ ID NOs: 67, 71, 82 and 85.

According to some embodiments of the invention, the signal generating moiety is encoded by a heterologous nucleic acid sequence.

According to some embodiments of the invention, the heterologous nucleic acid sequence encodes a polypeptide.

According to some embodiments of the invention, the signal generating moiety comprises a fluorescent moiety or a phosphorescent moiety.

According to some embodiments of the invention, the reporter polypeptide comprises a fluorescent moiety or a phosphorescent moiety.

According to some embodiments of the invention, the sample is a body fluid.

According to some embodiments of the invention, the body fluid is selected from the group consisting of saliva, blood, serum, milk and urine.

According to some embodiments of the invention, the sample is an environmental sample.

According to some embodiments of the invention, the method further comprises removing the ligand from the bacteria.

According to some embodiments of the invention, the removing is effected by contacting the bacteria with an RNA aptamer comprising a nucleic acid sequence as set forth in SEQ ID NOs: 45-88.

According to some embodiments of the invention, the transcription termination site is a premature transcription termination site.

According to some embodiments of the invention, the transcription termination site is a mature transcription termination site.

According to some embodiments of the invention, the ligand is selected from the group consisting of an antibiotic, a metabolite, a vitamin, an amino acid, a metal ion and a peptide.

According to some embodiments of the invention, the ligand controls the premature termination via a riboswitch or attenuator.

According to some embodiments of the invention, the bacteria are comprised in a heterogeneous population of bacteria.

According to some embodiments of the invention, the bacteria are comprised in a microbiome.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-H illustrate that term-seq maps RNA termini across the genome. A. Regulation by conditional termination in bacteria. The 5′ UTR shown contains a riboregulator (riboswitch, protein-binding leader or attenuator) that differentially folds to generate a condition-specific premature terminator. B. Schematic representation of the term-seq protocol. C. Mapping of term-seq reads to the genome yields a typical pattern where the majority of reads map to discrete intergenic positions marking RNA 3′ ends. Black arrows represent individual mapped reads. D. Reproducibility of the term-seq results. Data from three biological replicates over a representative 3 kb window of the B. subtilis genome is presented. Black arrowheads represent positions supported by term-seq reads, with arrow height (y-axis) representing the number of reads supporting the position. E. Multi-layered RNA sequencing data provides an integrative view of the bacterial transcriptome. Black arrowheads represent predicted term-seq termination sites, with arrow height indicating the average number of reads in three biological replicates. Black curve represents RNA-seq coverage. Red arrowheads mark the position of transcription start sites (TSSs), as inferred from transcriptome-wide sequencing of RNA 5′ ends^{31,32} (Methods). F. Folding energy of RNA termini predicted by term-seq (n=1489, green bars) compared with random intergenic sites (n=10,000, red bars). Energy was determined by running RNAfold³³ on the 40 bases immediately upstream to the site. G. Uridine-rich tail upstream to term-seq sites (n=1489). Plot generated by WebLogo³⁴. H. Assessment of the quantitative nature of term-seq using in-vitro synthesized RNA. The number of reads covering the exact ends of the ERCC transcripts (Materials and Methods) were counted and compared to the known concentration of the RNAs. Pearson correlation, R=0.98.

FIGS. 2A-I depict the discovery of genes regulated by conditional termination. A-B. Known riboswitches in B. subtilis display a typical pattern of premature termination in the 5′UTR. In both (A) Thiamine pyrophosphate (TPP) riboswitch and (B) Lysine riboswitch (cyan arrows) a term-seq site is observed downstream to the riboswitch. C. Known and novel regulators identified by applying term-seq on B. subtilis, L. monocytogenes and E. faecalis. Pie charts describe the number of regulators identified in each functional category and organism (Tables 3-5). D-I. Examples of novel regulatory elements (yellow arrows) identified in this study. Axes and colors are as in FIG. 1E. The secondary structures of the 5′UTR regulators were predicted using RNAfold³³. The TSS positions identified by 5′ end sequencing are marked by red arrows.

FIGS. 3A-C illustrate in vivo metabolite screening using RNA sequencing. A. Genome-wide experimental approach for in vivo screening of termination-based regulators that respond to a metabolite of choice in physiological conditions. A bacterium of interest is cultured in a defined medium with or without the metabolite of choice. After a brief incubation, RNA is extracted and sequenced using term-seq and RNA-seq. The long/short transcript ratio, indicative of the open/closed state of the regulator, can be calculated from term-seq or RNA-seq counts. B-C. RNA-seq was applied on B. subtilis grown in defined, minimal media either containing both lysine and methionine (black RNA-seq coverage), lacking lysine and containing methionine (green) or containing lysine and lacking methionine (red). RNA-seq levels for the two known lysine riboswitches are presented. RNA-seq coverage was normalized by the number of uniquely mapped reads in each sequencing library. Red arrows represent TSS positions identified by 5′ end sequencing.

FIGS. 4A-G present antibiotic responsive conditional terminators. The antibiotic-dependent response of known and novel regulators as measured in-vivo by term-seq and RNA-seq. Black, green and blue RNA-seq coverage and term-seq sites denote the control (LB), lincomycin, and erythromycin conditions, respectively. Term-seq sites represent average read coverage across 3 biological replicates. A. The B. subtilis bmrCD operon³⁹. B. The B. subtilis vmlR gene⁴¹. C-F. Antibiotic dependent transcriptional readthrough in novel regulators discovered in L. monocytogenes and E. faecalis. G. Condition-specific readthrough calculated in the control and the seven antibiotics exposure experiments. The antibiotic class is defined by the cellular process/component targeted. RNA-seq coverage was normalized by the number of uniquely mapped reads in each sequencing library. Red arrows mark TSS positions identified by 5′ end sequencing. Antibiotics and abbreviations used: Lincomycin (Lm), Erythromycin (Em), Chloramphenicol (Cap), Kanamycin (Km), Ofloxacin (Oflox), Bacitracin (Bac) and Ampicillin (Amp).

FIGS. 5A-H Antibiotic-responsive terminator/antiterminator RNA structures control the expression of lmo0919. Mutational analysis of the 5′UTR of lmo0919 provides insights into the mechanism of inducible antibiotic resistance. A. A model for the predicted RNA secondary structure of the lmo0919 5′ UTR. This element is predicted to form two alternative, mutually exclusive structures that mediate either termination or antibiotic-dependent readthrough. Left, the “closed-state” structure encodes a terminator and an upstream stem; right, the “open-state” structure in which the terminator structure is sequestered by an anti-terminator. B. Generation of mutants that interrupt the anti-anti-terminator (red), the anti-terminator (green), or a putative uORF that overlaps the anti-terminator (purple). C-D. Mutants were grown in BHI media without lincomycin (C) or containing 0.5 ug/ml lincomycin (D), respectively. Error bars represent standard error. E-H. Term-seq and RNA-seq coverage of WT and mutants grown in BHI without lincomycin (black RNA-seq curves and black term-seq sites) or with 0.5 ug/ml lincomycin (green RNA-seq curves and green term-seq sites). RNA-seq coverage was normalized by the number of uniquely mapped reads in each sequencing library.

FIG. 6 illustrates sequence and structure analysis of terminators predicted by term-seq. For each site, either term-seq predicted (n=1489) or randomly selected intergenic sites (n=10,000), the 40 bp preceding the site were folded in-silico using RNAfold, and the resulting folding energy was recorded. The number of uridine residues in the 8 nucleotides immediately upstream of the site was also recorded. Shown is a matrix presenting the joint distribution of terminators predicted by term-seq vs. randomly generated sites across the landscape of fold stability (kcal/mol, x-axis) and the number of uridines immediately upstream the site (y-axis). Term-seq logo represents all sites predicted by term-seq (n=1498) and the random site logo shows an equal set of randomly selected intergenic sites (n=1498).

FIG. 7 illustrates readthrough responses of B. subtilis regulators to lysine depletion. The known lysine riboswitches are the only regulators that increase their activity in response to lysine depletion in a statistically significant manner. The X and Y axes represent the percent readthrough (ratio of short to long transcripts) recorded in growth media either containing or depleted of the amino acid lysine, respectively. The plot shows regulators in which the gene was covered by a minimum of 50 Reads Per Kilo-base per Million (RPKM) in at least one condition (n=56). All regulators are denoted as blue circles except the known lysine responsive riboswitches, which are marked in red. Statistical significance of the lysine riboswitches response was tested using the Chi Square test of independence (P=1.32e⁻⁸° and P=8.91e⁻⁰⁴ for lysine riboswitch I and II, respectively).

FIG. 8 presents evidence of a translated uORF embedded within the vmlR regulator in B. subtilis. Previously published B. subtilis ribosome profiling data (Ref 56; purple coverage) was plotted alongside control RNA-seq and term-seq data (black coverage and black arrows, respectively). The vmlR regulator contains a highly covered ribosome footprint that overlaps a five amino-acid long potential uORF. The TSS position is marked by a red arrow.

FIGS. 9A-B illustrate antibiotic-responsive regulation in the human oral microbiome. The meta-term-seq approach facilitates the discovery of metabolite-responsive regulators across complex bacterial communities. (A) Schematics of the meta-term-seq workflow from sample collection to regulator identification. (B) A phylogenetic tree comprised of oral microbiome bacteria found to have one or more lincomycin-responsive regulators. The predicted functions of the regulated genes in each species are indicated by colored boxes according to the inset legend. In some cases a single operon contained several different functions (multi-colored rectangles, legend bottom). Individual bacteria studied in monoculture were added to the tree (marked by blue-colored names).

FIGS. 10A-F illustrate that sub-lethal exposure to antibiotics does not cause global changes in RNA expression or regulator activity. Antibiotic dependent regulation is not a result of global non-specific stress caused by antibiotic exposure. Shown is data for L. monocytogenes. (A-B) Genome-wide mRNA levels are not significantly affected by antibiotic sublethal treatment as shown by the extremely high correlation between antibiotic-treated and control samples. Genes supported by a minimum of 100 reads per million (RPM) are shown in the scatter plots (n=1851). (C) A correlation plot between all antibiotic-treated and untreated conditions. (D-F) Ribo-regulator activities of lmo0919 and lmo1652 are highly specific to antibiotic exposure. The scatter plots compare the read-through levels of treated vs. untreated samples. Color scheme represents the change in expression of the regulated gene.

FIG. 11 illustrates that the lmo0919 regulator senses ribosome inhibition. L. monocytogenes carrying the ErmC antibiotic resistance gene was assayed for lincomycin dependent induction. In the L. monocytogenes ErmC-expressing strain, in which ribosome are made immune to lincomycin via methylation of A2058 in the 23S rRNA, the lmo0919 regulator is unresponsive to the presence of the antibiotic (red).

FIG. 12 illustrates the conserved structural architecture of the lmo0919 regulator. The predicted RNA structures of the lmo0919 regulator and its homologues display a conserved anti-anti-terminator/antiterminator arrangement overlapped by a 3-amino-acid uORF. RNA sequences were folded with RNAfold and colored according to their base-pair probabilities.

FIG. 13 illustrates that the conserved μORF found in the lmo0919 regulator is translated in-vivo. The L. monocytogenes lmo0919 ribo-regulator (rli53) was modified by a chromosomal in-frame fusion of a GFP reporter protein that lacks the initiation codon, to the conserved 3aa uORF (MKF). Left and right panels show the phase contrast and fluorescence images, respectively, and demonstrate that the uORF is translated in-vivo.

FIG. 14 is a graph illustrating lincomycin-dependent ribosome stalling in the lmo0919 ribo-regulator. Ribosome profiling (Ribo-seq) of L. monocytogenes and the closely related L. innocua with or without a brief exposure to lincomycin reveals significant antibiotic-dependent ribosome stalling over the conserved uORF (MKF-stop) (Methods). The Y-axis shows the antibiotic-dependent enrichment in ribosome occupancy over the region spanning the uORF.

FIG. 15 illustrates antibiotic-dependent ribo-regulators discovered using meta-term-seq. The antibiotic-dependent response of novel regulators as measured in-vivo by RNA-seq. Control and lincomycin treated samples are shown in black and green, respectively. RNA-seq coverage was normalized by the number of uniquely mapped reads in each library.

### DESCRIPTION OF SPECIFIC EMBODIMENTS OF THE INVENTION

The present invention, in some embodiments thereof, relates to isolated polynucleotides that serve as bacterial transcription terminators, methods of identifying same and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have uncovered an unbiased experimental method for high-throughput discovery of conditional-termination-based regulators in bacteria. Moreover, they developed a screening procedure that measures the in-vivo read-through levels of every regulator in the genome in parallel, thus enabling the identification of regulators that specifically respond to a given metabolite. The power of this approach was demonstrated by detecting dozens of novel regulators in three bacteria, and identifying a subset of regulators that specifically respond to translation-inhibiting antibiotics.

The Examples section herein below provides evidence that the L. monocytogenes lmo0919 gene is a lincomycin-specific resistance gene, the expression of which is controlled by ribosome-dependent conditional termination that specifically responds to lincomycin antibiotics.

Finally, the present inventors experimentally demonstrate that this mode of regulation is highly abundant in human-associated bacteria and controls a variety of antibiotics-resistance gene classes.

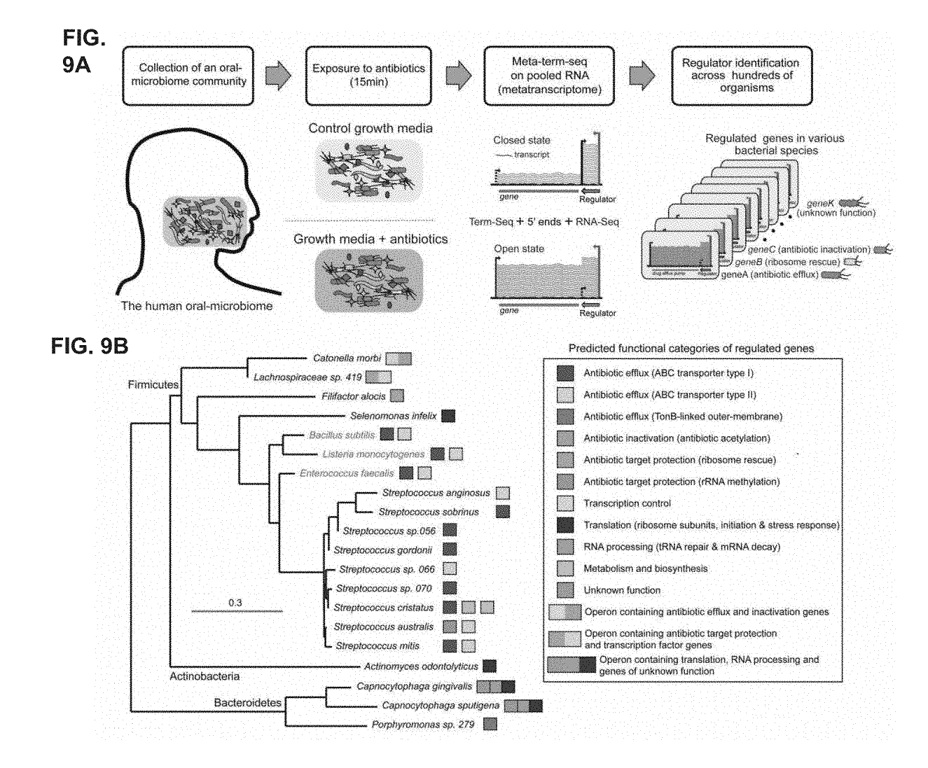

The basis of the approach exemplified herein, referred to herein as “term-seq” is a new RNA-sequencing protocol that quantitatively maps bacterial RNA 3′ ends to the single nucleotide resolution in a transcriptome-wide manner. As regulator identification via term-seq does not rely on comparative genomics, it can identify Glade-specific regulators that are not conserved across a wide array of organisms, as well as short regulators, both of which are generally challenging or even impossible to find when relying on sequence conservation. A unique advantage of term-seq is its ability to measure the in-vivo activity of every expressed regulator in the cell in parallel. Additionally, the measurements are recorded within the original, non-engineered locus, preventing experimental biases that stem from artificial transfer of the regulator to a model organism, and allowing for large scale screens/studies of regulators in organisms lacking genetic-engineering tools. By measuring the ratio of premature and full-length termination events, term-seq separates between promoter- and regulator-specific effects (as illustrated in FIGS. 4B and 4E) and thus permits the study of regulator types, in which complex regulatory logic is imposed by promoter-regulator combinations. Moreover, the present inventors show that application of term-seq on meta-transcriptomes (meta-term-seq) enables regulator identification in multiple organisms in parallel (as illustrated in FIGS. 9A-B).

According to a first aspect of the present invention there is provided a method of determining a transcription termination site in bacterial DNA. This method (referred to herein as term seq) comprises:

(a) ligating a first adaptor to the 3′ end of RNA transcripts of a bacterial RNA sample to generate elongated RNA transcripts;

(b) fragmenting the elongated RNA transcripts;

(c) combining the elongated RNA transcripts with a reverse transcriptase and an oligonucleotide that hybridizes to the adaptor under conditions that allow synthesis of cDNA from the elongated RNA transcripts;

(d) ligating a second adaptor to the 3′ end of the cDNA to generate elongated cDNA transcripts;

(e) amplifying the elongated cDNA transcripts using primers that hybridize to the first adaptor and the second adaptor to generate amplified DNA; and

(f) sequencing the amplified DNA, thereby determining the transcriptional termination site in bacterial DNA.

As used herein, the phrase “transcription termination site” refers to a nucleic acid sequence (or base) that marks the end of transcription of a gene. It will be appreciated that the transcription termination site may mark the end of a gene or operon, or may be a premature site present in the 5′ UTR of a gene (i.e. a premature transcription termination site).

Both gram positive and gram negative bacteria may be analyzed according to this aspect of the present invention.

The term “Gram-positive bacteria” as used herein refers to bacteria characterized by having as part of their cell wall structure peptidoglycan as well as polysaccharides and/or teichoic acids and are characterized by their blue-violet color reaction in the Gram-staining procedure. Representative Gram-positive bacteria include: Actinomyces spp., Bacillus anthracis, Bifidobacterium spp., Clostridium botulinum, Clostridium perfringens, Clostridium spp., Clostridium tetani, Corynebacterium diphtheriae, Corynebacterium jeikeium, Enterococcus faecalis, Enterococcus faecium, Erysipelothrix rhusiopathiae, Eubacterium spp., Gardnerella vaginalis, Gemella morbillorum, Leuconostoc spp., Mycobacterium abcessus, Mycobacterium avium complex, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, Nocardia spp., Peptococcus niger, Peptostreptococcus spp., Proprionibacterium spp., Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A streptococcus), Streptococcus salivarius, Streptococcus sanguis.

The term “Gram-negative bacteria” as used herein refer to bacteria characterized by the presence of a double membrane surrounding each bacterial cell. Representative Gram-negative bacteria include Acinetobacter calcoaceticus, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chalmydia pneumoniae, Chlamydia psittaci, Chlamydia trachomatis, Chromobacterium violaceum, Citrobacter spp., Eikenella corrodens, Enterobacter aerogenes, Escherichia coli, Flavobacterium meningosepticum, Fusobacterium spp., Haemophilus influenzae, Haemophilus spp., Helicobacter pylori, Klebsiella spp., Legionella spp., Leptospira spp., Moraxella catarrhalis, Morganella morganii, Mycoplasma pneumoniae, Neisseria gonorrhoeae, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaea spp., Salmonella spp., Salmonella typhi, Serratia marcescens, Shigella spp., Treponema carateum, Treponema pallidum, Treponema pallidum endemicum, Treponema pertenue, Veillonella spp., Vibrio cholerae, Vibrio vulnificus, Yersinia enterocolitica, Yersinia pestis.

The steps of the method of this aspect of the present invention will be described individually:

(a) Ligating a First Adaptor to the 3′ End of RNA Transcripts of a Bacterial RNA Sample to Generate Elongated RNA Transcripts:

The RNA sample may be derived from a population of bacterial cells or from a single cell. The population may be a population of a single bacteria type or a mixed population (heterogeneous population) or two or more bacteria types. According to a particular embodiment the RNA sample is a microbiome sample, as further described herein below. The RNA may comprise total RNA, mRNA, mitochondrial RNA, chloroplast RNA, viral RNA, cell free RNA, and/or mixtures thereof.

Methods of isolating RNA, particularly messenger RNA (mRNA) are well known to those skilled in the art. Typically, cell disruption is performed in the presence of strong protein denaturing solutions, which inactivate RNAses during the RNA isolation procedure. RNA is then isolated using differential ethanol precipitation with centrifugation.

Typically the RNA sample is devoid of DNA. DNA may be removed from the RNA sample using a DNAse enzyme.

The first adaptor is a single stranded oligonucleotide of between 5-100 nucleotides, more preferably between 10-80 nucleotides, more preferably between 20-60 nucleotides.

The adapter may comprise sequences recognizable by a PCR primer, sequences which are necessary for attaching to a flow cell surface (P5 and P7 sites), a sequence which encodes for a promoter for an RNA polymerase and/or a restriction site.

According to a particular embodiment, the adaptor is chemically modified (e.g., 5′ phosphorylated and/or 3′ amino blocked). An exemplary sequence of the adaptor is set forth in SEQ ID NO: 90.

Ligation is carried out using a ligase enzyme (e.g., T4 or T3 ligase) under conditions (e.g., temperature, buffer, salt, ionic strength, and pH conditions) that allow ligation of the adapter polynucleotide to the RNA molecules.

(b) Fragmenting the Elongated RNA Transcripts:

Physical fragmentation methods contemplated by the present invention include acoustic shearing, sonication or hydrodynamic shearing. Chemical fragmentation involves the use of heat and divalent metal cations.

(c) Combining the Elongated RNA Transcripts with a Reverse Transcriptase (RT) and an Oligonucleotide that Hybridizes to Said Adaptor Under Conditions that Allow Synthesis of cDNA from Said Elongated RNA Transcripts:

RTs are well known in the art. Examples of RTs include, but are not limited to, Moloney murine leukemia virus (M-MLV) reverse transcriptase, human immunodeficiency virus (HIV) reverse transcriptase, rous sarcoma virus (RSV) reverse transcriptase, avian myeloblastosis virus (AMV) reverse transcriptase, rous associated virus (RAV) reverse transcriptase, and myeloblastosis associated virus (MAV) reverse transcriptase or other avian sarcoma-leukosis virus (ASLV) reverse transcriptases, and modified RTs derived therefrom. See e.g., U.S. Pat. No. 7,056,716.

Additional components required in a reverse transcription reaction include dNTPS (dATP, dCTP, dGTP and dTTP) and optionally a reducing agent such as Dithiothreitol (DTT) and MnCl₂.

(d) Ligating a Second Adaptor to the 3′ End of Said cDNA to Generate Elongated cDNA Transcripts:

The second adaptor is now ligated to the cDNA using a ligase enzyme (as described herein above). The second adaptor is single-stranded and typically is an oligonucleotide of between 5-100 nucleotides, more preferably between 10-80 nucleotides, more preferably between 20-60 nucleotides.

The second adapter may comprise sequences recognizable by a PCR primer, sequences which are necessary for attaching to a flow cell surface (e.g., P5 and P7 sites), a sequence which encodes for a promoter for an RNA polymerase and/or a restriction site.

According to a particular embodiment, the adaptor is chemically modified (e.g., 5′ phosphorylated and/or 3′ amino blocked). An exemplary sequence of the adaptor is set forth in SEQ ID NO: 92.

(e) Amplifying the Elongated cDNA Transcripts Using Primers that Hybridize to the Sequence of the First Adaptor and the Sequence of the Second Adaptor to Generate Amplified DNA:

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR) and ligase chain reaction (LCR). In a typical PCR amplification reaction, a nucleic acid sequence of interest is often amplified at least fifty thousand fold in amount over its amount in the starting sample. A “copy” or “amplicon” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification.

A typical amplification reaction is carried out by contacting a forward and reverse primer (a primer pair) to the elongated cDNA described herein together with any additional amplification reaction reagents under conditions which allow amplification of the target sequence.

The terms “forward primer” and “forward amplification primer” are used herein interchangeably, and refer to a primer that hybridizes (or anneals) to the target (template strand).

The terms “reverse primer” and “reverse amplification primer” are used herein interchangeably, and refer to a primer that hybridizes (or anneals) to the complementary target strand. The forward primer hybridizes with the target sequence 5′ with respect to the reverse primer.

The term “amplification conditions”, as used herein, refers to conditions that promote annealing and/or extension of primer sequences. Such conditions are well-known in the art and depend on the amplification method selected. Thus, for example, in a PCR reaction, amplification conditions generally comprise thermal cycling, i.e., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and temperature cycling, buffer, salt, ionic strength, and pH, and the like.

As used herein, the term “amplification reaction reagents”, refers to reagents used in nucleic acid amplification reactions and may include, but are not limited to, buffers, reagents, enzymes having reverse transcriptase and/or polymerase activity or exonuclease activity, enzyme cofactors such as magnesium or manganese, salts, nicotinamide adenine dinuclease (NAD) and deoxynucleoside triphosphates (dNTPs), such as deoxyadenosine triphosphate, deoxyguanosine triphosphate, deoxycytidine triphosphate and thymidine triphosphate. Amplification reaction reagents may readily be selected by one skilled in the art depending on the amplification method used.

According to this aspect of the present invention, the amplifying may be effected using techniques such as polymerase chain reaction (PCR), which includes, but is not limited to Allele-specific PCR, Assembly PCR or Polymerase Cycling Assembly (PCA), Asymmetric PCR, Helicase-dependent amplification, Hot-start PCR, Intersequence-specific PCR (ISSR), Inverse PCR, Ligation-mediated PCR, Methylation-specific PCR (MSP), Miniprimer PCR, Multiplex Ligation-dependent Probe Amplification, Multiplex-PCR, Nested PCR, Overlap-extension PCR, Quantitative PCR (Q-PCR), Reverse Transcription PCR (RT-PCR), Solid Phase PCR: encompasses multiple meanings, including Polony Amplification (where PCR colonies are derived in a gel matrix, for example), Bridge PCR (primers are covalently linked to a solid-support surface), conventional Solid Phase PCR (where Asymmetric PCR is applied in the presence of solid support bearing primer with sequence matching one of the aqueous primers) and Enhanced Solid Phase PCR (where conventional Solid Phase PCR can be improved by employing high Tm and nested solid support primer with optional application of a thermal ‘step’ to favour solid support priming), Thermal asymmetric interlaced PCR (TAIL-PCR), Touchdown PCR (Step-down PCR), PAN-AC and Universal Fast Walking.

The PCR (or polymerase chain reaction) technique is well-known in the art and has been disclosed, for example, in K. B. Mullis and F. A. Faloona, Methods Enzymol., 1987, 155: 350-355 and U.S. Pat. Nos. 4,683,202; 4,683,195; and 4,800,159 (each of which is incorporated herein by reference in its entirety). In its simplest form, PCR is an in vitro method for the enzymatic synthesis of specific DNA sequences, using two oligonucleotide primers that hybridize to opposite strands and flank the region of interest in the target DNA. A plurality of reaction cycles, each cycle comprising: a denaturation step, an annealing step, and a polymerization step, results in the exponential accumulation of a specific DNA fragment (“PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al., (Eds.), 1991, IRL Press: Oxford; R. K. Saiki et al., Nature, 1986, 324: 163-166). The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs).

(f) Sequencing the Amplified DNA:

The DNA may be sequenced using any method known in the art—e.g., massively parallel DNA sequencing, sequencing-by-synthesis, sequencing-by-ligation, 454 pyrosequencing, cluster amplification, bridge amplification, and PCR amplification, although preferably, the method comprises deep sequencing using a high throughput sequencing method.

As used herein, the term “deep sequencing” and variations thereof refers to the number of times a nucleotide is read during the sequencing process. Deep sequencing indicates that the coverage, or depth, of the process is many times larger than the length of the sequence under study.

Exemplary methods include the sequencing technology and analytical instrumentation offered by Roche 454 Life Sciences™, Branford, Conn., which is sometimes referred to herein as “454 technology” or “454 sequencing.”; the sequencing technology and analytical instrumentation offered by Illumina, Inc, San Diego, Calif. (their Solexa Sequencing technology is sometimes referred to herein as the “Solexa method” or “Solexa technology”); or the sequencing technology and analytical instrumentation offered by ABI, Applied Biosystems, Indianapolis, Ind., which is sometimes referred to herein as the ABI-SOLiD™ platform or methodology.

Other known methods for sequencing include, for example, those described in: Sanger, F. et al., Proc. Natl. Acad. Sci. U.S.A. 75, 5463-5467 (1977); Maxam, A. M. & Gilbert, W. Proc Natl Acad Sci USA 74, 560-564 (1977); Ronaghi, M. et al., Science 281, 363, 365 (1998); Lysov, 1. et al., Dokl Akad Nauk SSSR 303, 1508-1511 (1988); Bains W. & Smith G. C. J. Theor Biol 135, 303-307 (1988); Drnanac, R. et al., Genomics 4, 114-128 (1989); Khrapko, K. R. et al., FEBS Lett 256.118-122 (1989); Pevzner P. A. J Biomol Struct Dyn 7, 63-73 (1989); and Southern, E. M. et al., Genomics 13, 1008-1017 (1992). Pyrophosphate-based sequencing reaction as described, e.g., in U.S. Pat. Nos. 6,274,320, 6,258,568 and 6,210,891, may also be used.

Following sequencing, the DNA may be aligned with bacterial genomes to determine the position of the terminal nucleotide on the genome.

To avoid reads that represent degraded RNA, the method may be performed in replicates and positions that are independently reproduced in each of the replicates (e.g., 2, 3, 4, or 5) may be considered. Further, only when at least a statistically significant number of reads (e.g., 3, 4, 5 or more) of a termination site are covered, in some embodiments is the termination site considered to be a true termination site.

Alignment may be effected using known computer programs including for BLAST, NovoAlig or Bowtie2.

It will be appreciated that steps (c) and (d) may be reversed such that the ligation of the second adaptor is performed prior to the reverse transcription step. In this embodiment, the second adaptor is ligated to the 5′ end of the RNA transcript and not to the cDNA.

Using the above described method, the present inventors showed it is possible to screen for ligands which are capable of controlling premature transcription termination of bacterial genes.

Thus, according to another aspect of the present invention there is provided a method of determining whether a ligand can control premature transcription termination of a bacterial gene comprising:

(a) culturing bacteria in a medium comprising the ligand;

(b) analyzing the number of full length RNA transcripts transcribed from the bacterial gene and the number of prematurely terminated RNA transcripts transcribed from the bacterial gene; and

(c) comparing the ratio of prematurely terminated RNA transcripts transcribed from the bacterial gene: full length RNA transcripts transcribed from the bacterial gene in the presence of the ligand to the ratio of prematurely terminated RNA transcripts transcribed from the bacterial gene: full length RNA transcripts transcribed from the bacterial gene in the absence of the ligand, wherein a statistically significant change in said ratio is indicative that the ligand can control premature transcription termination of the bacterial gene.

Bacteria which may be cultured according to this aspect of the present invention include both gram positive and gram negative bacteria as described herein above. It will be appreciated that the bacteria that are cultured may be comprised in a homogeneous population (i.e. a single bacteria type) or comprised in a heterogeneous population (i.e. comprise a plurality of bacteria types). According to a particular embodiment, a sample of a microbiome is cultured.

As used herein, the term “microbiome” refers to the totality of microbes (bacteria, fungae, protists), their genetic elements (genomes) in a defined environment. The microbiome may be a gut microbiome, an oral microbiome, a bronchial microbiome, a skin microbiome or a vaginal microbiome.

The bacteria may be cultured in any medium that allows the bacteria to remain viable and propagate (e.g., LB, TB, Brain Heart Infusion (BHI) broth (Difco), or M9 minimal media).

Exemplary ligands that may be tested include, but are not limited to antibiotics, metabolites (e.g., bacterial metabolites), vitamins, amino acids, metal ions and peptides.

The term “antibiotic” is used herein to describe a compound or composition which decreases the viability of a microorganism, or which inhibits the growth or reproduction of a microorganism. As used in this disclosure, an antibiotic is further intended to include an antimicrobial, bacteriostatic, or bactericidal agent. Exemplary antibiotics include, but are not limited to, penicillins, cephalosporins, penems, carbapenems, monobactams, aminoglycosides, sulfonamides, macrolides, tetracyclines, lincosides, quinolones, chloramphenicol, vancomycin, metronidazole, rifampin, isoniazid, spectinomycin, trimethoprim, sulfamethoxazole, and the like.

Particular examples of antibiotics include, but are not limited to lincomycin, erythromycin, chloramphenicol, kanamycin, ofloxacin, ampicilin, tylosin and bacitracin.

According to a particular embodiment, the ligand is capable of penetrating a bacterial cell.

According to another embodiment, the ligand is capable of controlling premature transcription termination via a riboswitch (i.e. direct binding of the ligand to the RNA molecule, not dependent on ribosome activity).

According to another embodiment, the ligand is capable of controlling premature transcription termination via attenuation (i.e. dependent on ribosome activity).

Analyzing the number of full length RNA transcripts transcribed from the bacterial gene and the number of prematurely terminated RNA transcripts transcribed from the bacterial gene may be effected as described herein above (i.e. by term seq). According to a particular embodiment the prematurely terminated RNA transcripts are terminated at a position in their 5′UTR.

As well as performing term seq (as described herein above), the present inventors contemplate using other RNA sequencing methods known in the art.

Commercial kits are available for such a purpose—e.g., those manufactured by New England Biolabs—(www(dot)neb(dot)com/products/e7420-nebnext-ultra-directional-ma-library-prep-kit-for-illumina).

The ratio of prematurely terminated RNA transcripts transcribed from the bacterial gene: full length RNA transcripts transcribed from the bacterial gene in the absence of the ligand may be determined following to, prior to or concomitantly with the determining of the ratio of prematurely terminated RNA transcripts transcribed from the bacterial gene: full length RNA transcripts transcribed from the bacterial gene is measured in the presence of the ligand. Preferably, the ratio (in the absence of the ligand) is determined under the same experimental conditions that are used when determining the ratio in the presence of the ligand (e.g., cultured in the same medium, at the same temperature etc). Alternatively, the ratio of prematurely terminated RNA transcripts transcribed from the bacterial gene: full length RNA transcripts transcribed from the bacterial gene in the absence of the ligand may be already known and need not be experimentally determined (i.e. a known reference value).

According to one embodiment, when the level of premature transcription termination of a particular gene in the bacteria is increased by at least 1.5 fold, 2 fold, 4 fold, 5 fold, 10 fold or 20 fold in the presence of the ligand as compared to the level of premature transcription termination of that gene in the bacteria in the absence of the ligand, the ligand is referred to as one which is capable of upregulating premature transcription termination.

According to another embodiment, when the level of premature transcription termination of a particular gene in the bacteria is decreased by at least 1.5 fold, 2 fold, 4 fold, 5 fold, 10 fold or 20 fold in the presence of the ligand as compared to the level of premature transcription termination of that gene in the bacteria in the absence of the ligand, the ligand is referred to as one which is capable of downregulating premature transcription termination.

In an exemplary embodiment, the method can be used to detect whether a sample comprises an antibiotic. The present inventors showed that the regulatory elements discovered for lmo0919 (SEQ ID NO: 23) and EF2720 (SEQ ID NO: 41) respond to lincomycin, whereas the regulatory elements EF1413 (SEQ ID NO: 38) and lmo1652 (SEQ ID NO: 27) respond to other antibiotics including erythromycin, chloramphenicol, kanamycin, ofloxacin, ampicilin, tylosin and bacitracin; and lmo1652 (SEQ ID NO: 27) responds to lincomycin, erythromycin and chloramphenicol.

The method comprises:

(a) culturing a L. monocytogenes or E. faecalis bacteria in a medium comprising said sample;

(b) analyzing the number of full length RNA transcripts transcribed from the bacterial gene selected from the group consisting of lmo0919, lmo1652, EF1413 and EF2720 and prematurely terminated RNA transcripts transcribed from said bacterial gene; and

(c) comparing the ratio of prematurely terminated RNA transcripts transcribed from said bacterial gene: full length RNA transcripts transcribed from said bacterial gene in the presence of the sample to the ratio of prematurely terminated RNA transcripts transcribed from said bacterial gene: full length RNA transcripts transcribed from said bacterial gene in the absence of the sample, wherein a statistically significant change in said ratio is indicative that the sample comprises an antibiotic.

As mentioned herein above, using the above described methods, the present inventors identified 44 novel regulatory elements which serve as riboswitches or attenuators which may be triggered at or above threshold levels of the trigger molecules (i.e. ligands). Such regulatory elements can be used to control downstream transcription of a heterologous nucleic acid sequence.

Thus, according to another aspect of the present invention there is provided an isolated polynucleotide comprising a nucleic acid sequence as set forth in SEQ ID NOs: 1-44 operatively linked to a heterologous nucleic acid sequence.

As used herein, the qualifier “heterologous” when relating to heterologous nucleic acid sequence indicates that the nucleic acid is not naturally found operatively linked to the regulatory elements when they are in their biological genomic environment.

In one embodiment, the heterologous nucleic acid sequence encodes a polypeptide or a fragment thereof.

Contemplated polypeptides are ones that are endogenous or exogenous to the host cell in which they are being expressed. The polypeptides may be intracellular polypeptides (e.g., a cytosolic protein), transmembrane polypeptides, or secreted polypeptides. Heterologous production of proteins is widely employed in research and industrial settings, for example, for production of therapeutics, vaccines, diagnostics, biofuels, and many other applications of interest. Exemplary therapeutic proteins that can be produced by employing the subject compositions and methods include but are not limited to certain native and recombinant human hormones (e.g., insulin, growth hormone, insulin-like growth factor 1, follicle-stimulating hormone, and chorionic gonadotropin), hematopoietic proteins (e.g., erythropoietin, C-CSF, GM-CSF, and IL-11), thrombotic and hematostatic proteins (e.g., tissue plasminogen activator and activated protein C), immunological proteins (e.g., interleukin), antibodies and other enzymes (e.g., deoxyribonuclease I). Exemplary vaccines that can be produced by the subject compositions and methods include but are not limited to vaccines against various influenza viruses (e.g., types A, B and C and the various serotypes for each type such as H5N2, H1N1, H3N2 for type A influenza viruses), HIV, hepatitis viruses (e.g., hepatitis A, B, C or D), Lyme disease, and human papillomavirus (HPV). Examples of heterologously produced protein diagnostics include but are not limited to secretin, thyroid stimulating hormone (TSH), HIV antigens, and hepatitis C antigens.

Proteins or peptides produced by the heterologous polypeptides can include, but are not limited to cytokines, chemokines, lymphokines, ligands, receptors, hormones, enzymes, antibodies and antibody fragments, and growth factors. Non-limiting examples of receptors include TNF type I receptor, IL-1 receptor type II, IL-1 receptor antagonist, IL-4 receptor and any chemically or genetically modified soluble receptors. Examples of enzymes include acetlycholinesterase, lactase, activated protein C, factor VII, collagenase (e.g., marketed by Advance Biofactures Corporation under the name Santyl); agalsidase-beta (e.g., marketed by Genzyme under the name Fabrazyme); dornase-alpha (e.g., marketed by Genentech under the name Pulmozyme); alteplase (e.g., marketed by Genentech under the name Activase); pegylated-asparaginase (e.g., marketed by Enzon under the name Oncaspar); asparaginase (e.g., marketed by Merck under the name Elspar); and imiglucerase (e.g., marketed by Genzyme under the name Ceredase). Examples of specific polypeptides or proteins include, but are not limited to granulocyte macrophage colony stimulating factor (GM-CSF), granulocyte colony stimulating factor (G-CSF), macrophage colony stimulating factor (M-CSF), colony stimulating factor (CSF), interferon beta (IFN-β), interferon gamma (IFN γ), interferon gamma inducing factor I (IGIF), transforming growth factor beta (IGF-β), RANTES (regulated upon activation, normal T-cell expressed and presumably secreted), macrophage inflammatory proteins (e.g., MIP-1-α and MIP-1-β), Leishmnania elongation initiating factor (LEIF), platelet derived growth factor (PDGF), tumor necrosis factor (TNF), growth factors, e.g., epidermal growth factor (EGF), vascular endothelial growth factor (VEGF), fibroblast growth factor, (FGF), nerve growth factor (NGF), brain derived neurotrophic factor (BDNF), neurotrophin-2 (NT-2), neurotrophin-3 (NT-3), neurotrophin-4 (NT-4), neurotrophin-5 (NT-5), glial cell line-derived neurotrophic factor (GDNF), ciliary neurotrophic factor (CNTF), TNF alpha type II receptor, erythropoietin (EPO), insulin and soluble glycoproteins e.g., gp120 and gp160 glycoproteins. The gp120 glycoprotein is a human immunodeficiency virus (WIV) envelope protein, and the gp160 glycoprotein is a known precursor to the gp120 glycoprotein. Other examples include secretin, nesiritide (human B-type natriuretic peptide (hBNP)) and GYP-I.

Other heterologous products may include GPCRs, including, but not limited to Class A Rhodopsin like receptors such as Muscatinic (Muse.) acetylcholine Vertebrate type 1, Musc. acetylcholine Vertebrate type 2, Musc. acetylcholine Vertebrate type 3, Musc. acetylcholine Vertebrate type 4; Adrenoceptors (Alpha Adrenoceptors type 1, Alpha Adrenoceptors type 2, Beta Adrenoceptors type 1, Beta Adrenoceptors type 2, Beta Adrenoceptors type 3, Dopamine Vertebrate type 1, Dopamine Vertebrate type 2, Dopamine Vertebrate type 3, Dopamine Vertebrate type 4, Histamine type 1, Histamine type 2, Histamine type 3, Histamine type 4, Serotonin type 1, Serotonin type 2, Serotonin type 3, Serotonin type 4, Serotonin type 5, Serotonin type 6, Serotonin type 7, Serotonin type 8, other Serotonin types, Trace amine, Angiotensin type 1, Angiotensin type 2, Bombesin, Bradykffin, C5a anaphylatoxin, Finet-leu-phe, APJ like, Interleukin-8 type A, Interleukin-8 type B, Interleukin-8 type others, C-C Chemokine type 1 through type 11 and other types, C-X-C Chemokine (types 2 through 6 and others), C-X3-C Chemokine, Cholecystokinin CCK, CCK type A, CCK type B, CCK others, Endothelin, Melanocortin (Melanocyte stimulating hormone, Adrenocorticotropic hormone, Melanocortin hormone), Duffy antigen, Prolactin-releasing peptide (GPR10), Neuropeptide Y (type 1 through 7), Neuropeptide Y, Neuropeptide Y other, Neurotensin, Opioid (type D, K, M, X), Somatostatin (type 1 through 5), Tachykinin (Substance P (NK1), Substance K (NK2), Neuromedin K (NK3), Tachykinin like 1, Tachykinin like 2, Vasopressin/vasotocin (type 1 through 2), Vasotocin, Oxytocin/mesotocin, Conopressin, Galanin like, Proteinase-activated like, Orexin & neuropeptides FF, QRFP, Chemokine receptor-like, Neuromedin U like (Neuromedin U, PRXamide), hormone protein (Follicle stimulating hormone, Lutropin-choriogonadotropic hormone, Thyrotropin, Gonadotropin type I, Gonadotropin type II), (Rhod)opsin, Rhodopsin Vertebrate (types 1-5), Rhodopsin Vertebrate type 5, Rhodopsin Arthropod, Rhodopsin Arthropod type 1, Rhodopsin Arthropod type 2, Rhodopsin Arthropod type 3, Rhodopsin Mollusc, Rhodopsin, Olfactory (Olfactory 11 fam 1 through 13), Prostaglandin (prostaglandin E2 subtype EP 1, Prostaglandin E2/D2 subtype EP2, prostaglandin E2 subtype EP3, Prostaglandin E2 subtype EP4, Prostaglandin F2-alpha, Prostacyclin, Thromboxane, Adenosine type 1 through 3, Purinoceptors, Purinoceptor P2RY1-4,6,11 GPR91, Purinoceptor P2RY5,8,9,10 GPR35,92,174, Purinoceptor P2RY12-14 GPR87 (JDP-Glucose), Cannabinoid, Platelet activating factor, Gonadotropin-releasing hormone, Gonadotropin-releasing hormone type I, Gonadotropin-releasing hormone type II, Adipokinetic hormone like, Corazonin, Thyrotropin-releasing hormone & Secretagogue, Thyrotropin-releasing hormone, Growth hormone secretagogue, Growth hormone secretagogue like, Ecdysis-triggering hormone (ETHR), Melatonin, Lysosphingolipid & LPA (EDG), Sphingosine 1-phosphate Edg-1, Lysophosphatidic acid Edg-2, Sphingosine 1-phosphate Edg-3, Lysophosphatidic acid Edg4, Sphingosine 1-phosphate Edg-5, Sphingosine 1-phosphate Edg-6, Lysophosphatidic acid Edg-7, Sphingosine 1-phosphate Edg-8, Edg Other Leukotriene B4 receptor, Leukotriene B4 receptor BLT1, Leukotriene B4 receptor BLT2, Class A Orphan/other, Putative neurotransmitters, SREB, Mas proto-oncogene & Mas-related (MRGs), GPR45 like, Cysteinyl leukotriene, G-protein coupled bile acid receptor, Free fatty acid receptor (GP40, GP41, GP43), Class B Secretin like, Calcitonin, Corticotropin releasing factor, Gastric inhibitory peptide, Glucagon, Growth hormone-releasing hormone, Parathyroid hormone, PACAP, Secretin, Vasoactive intestinal polypeptide, Latrophilin, Latrophilin type 1, Latrophilin type 2, Latrophilin type 3, ETL receptors, Brain-specific angiogenesis inhibitor (BAI), Methuselah-like proteins (MTH), Cadherin EGF LAG (CELSR), Very large G-protein coupled receptor, Class C Metabotropic glutamate/pheromone, Metabotropic glutamate group I through III, Calcium-sensing like, Extracellular calcium-sensing, Pheromone, calcium-sensing like other, Putative pheromone receptors, GABA-B, GABA-B subtype 1, GABA-B subtype 2, GABA-B like, Orphan GPRC5, Orphan GPCR6, Bride of sevenless proteins (BOSS), Taste receptors (TiR), Class D Fungal pheromone, Fungal pheromone A-Factor like (STE2,STE3), Fungal pheromone B like (BAR,BBR,RCB,PRA), Class E cAMP receptors, Ocular albinism proteins, Frizzled/Smoothened family, frizzled Group A (Fz 1&2&4&5&7-9), frizzled Group B (Fz 3 & 6), fizzled Group C (other), Vomeronasal receptors, Nematode chemoreceptors, Insect odorant receptors, and Class Z Archaeal/bacterial/fungal opsins.

Bioactive peptides may also be produced by the heterologous sequences of the present invention. Examples include: BOTOX, Myobloc, Neurobloc, Dysport (or other serotypes of botulinum neurotoxins), alglucosidase alfa, daptomycin, YH-16, choriogonadotropin alfa, filgrastim, cetrorelix, interleukin-2, aldesleukin, teceleulin, denileukin diftitox, interferon alfa-n3 (injection), interferon alfa-n1, DL-8234, interferon, Suntory (gamma-1a), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alfa, collagenase, carperitide, recombinant human epidermal growth factor (topical gel, wound healing), DWP401, darbepoetin alfa, epoetin omega, epoetin beta, epoetin alfa, desirudin, lepirudin, bivalirudin, nonacog alpha, Mononine, eptacog alfa (activated), recombinant Factor VIII+VWF, Recombinate, recombinant Factor VIII, Factor VIII (recombinant), Alphnmate, octocog alfa, Factor VIII, palifermin, Indikinase, tenecteplase, alteplase, pamiteplase, reteplase, nateplase, monteplase, follitropin alfa, rFSH, hpFSH, micafungin, pegfilgrastim, lenograstim, nartograstim, sermorelin, glucagon, exenatide, pramlintide, iniglucerase, galsulfase, Leucotropin, molgramostim, triptorelin acetate, histrelin (subcutaneous implant, Hydron), deslorelin, histrelin, nafarelin, leuprolide sustained release depot (ATRIGEL), leuprolide implant (DUROS), goserelin, somatropin, Eutropin, KP-102 program, somatropin, somatropin, mecasermin (growth failure), enlfavirtide, Org-33408, insulin glargine, insulin glulisine, insulin (inhaled), insulin lispro, insulin deternir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex′ recombinant human luteinizing hormone, dornase alfa, trafermin, ziconotide, taltirelin, diboterminalfa, atosiban, becaplermin, eptifibatide, Zemaira, CTC-111, Shanvac-B, HPV vaccine (quadrivalent), octreotide, lanreotide, ancestirn, agalsidase beta, agalsidase alfa, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin III, Granditropin, Vitrase, recombinant insulin, interferon-alpha (oral lozenge), GEM-21S, vapreotide, idursulfase, omnapatrilat, recombinant serum albumin, certolizumab pegol, glucarpidase, human recombinant C1 esterase inhibitor (angioedema), lanoteplase, recombinant human growth hormone, enfuvirtide (needle-free injection, Biojector 2000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alfa-n3 (oral), belatacept, transdermal insulin patches, AMG-531, MBP-8298, Xerecept, opebacan, AIDSVAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMlD, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-1, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 1311-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, AOD-9604, linaclotid eacetate, CETi-1, Hemospan, VAL (injectable), fast-acting insulin (injectable, Viadel), intranasal insulin, insulin (inhaled), insulin (oral, eligen), recombinant methionyl human leptin, pitrakinra subcutancous injection, eczema), pitrakinra (inhaled dry powder, asthma), Multikine, RG-1068, MM-093, NBI-6024, AT-001, PI-0824, Org-39141, Cpn10 (autoimmune diseases/inflammation), talactoferrin (topical), rEV-131 (ophthalmic), rEV-131 (respiratory disease), oral recombinant human insulin (diabetes), RPI-78M, oprelvekin (oral), CYT-99007 CTLA4-Ig, DTY-001, valategrast, interferon alfa-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4x, HIV vaccine (oral), HPV vaccine, Tat Toxoid, YSPSL, CHS-13340, PTH(1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague F1V vaccine, AG-702, OxSODrol, rBetV1, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, reticulose, rGRF, P1A, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 111In-hEGF, AE-37, trasnizumab-DM1, Antagonist G, IL-12 (recombinant), PM-02734, IMP-321, rhIGF-BP3, BLX-883, CUV-1647 (topical), L-19 based radioimmunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-11031, peptide YY [3-36] (obesity, intranasal), FGLL, atacicept, BR3-Fc, BN-003, BA-058, human parathyroid hormone 1-34 (nasal, osteoporosis), F-18-CCR1, AT-1100 (celiac disease/diabetes), JPD-003, PTH(7-34) liposomal cream (Novasome), duramycin (ophthalmic, dry eye), CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix (immediate release), EP-51216, hGH (controlled release, Biosphere), OGP-I, sifuvirtide, TV4710, ALG-889, Org-41259, rhCC10, F-991, thymopentin (pulmonary diseases), r(m)CRP, hepatoselective insulin, subalin, L19-IL-2 fusion protein, elafin, NMK-150, ALTU-139, EN-122004, rhTPO, thrombopoietin receptor agonist (thrombocytopenic disorders), AL-108, AL-208, nerve growth factor antagonists (pain), SLV-317, CGX-1007, INNO-105, oral teriparatide (eligen), GEM-OS1, AC-162352, PRX-302, LFn-p24 fusion vaccine (Therapore), EP-1043, S pneumoniae pediatric vaccine, malaria vaccine, Neisseria meningitidis Group B vaccine, neonatal group B streptococcal vaccine, anthrax vaccine, HCV vaccine (gpE1+gpE2+MF-59), otitis media therapy, HCV vaccine (core antigen+ISCOMATRIX), hPTH(1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, and TP-9201.

In certain embodiments, the heterologously produced protein is an enzyme or biologically active fragments thereof. Suitable enzymes include but are not limited to: oxidoreductases, transferases, hydrolases, lyases, isomerases, and ligases. In certain embodiments, the heterologously produced protein is an enzyme of Enzyme Commission (EC) class 1, for example an enzyme from any of EC 1.1 through 1.21, or 1.97. The enzyme can also be an enzyme from EC class 2, 3, 4, 5, or 6. For example, the enzyme can be selected from any of EC 2.1 through 2.9, EC 3.1 to 3.13, EC 4.1 to 4.6, EC 4.99, EC 5.1 to 5.11, EC 5.99, or EC 6.1-6.6.

As used herein, the term “antibody” refers to a substantially intact antibody molecule.

As used herein, the phrase “antibody fragment” refers to a functional fragment of an antibody (such as Fab, F(ab′)2, Fv or single domain molecules such as VH and VL) that is capable of binding to an epitope of an antigen.

According to one embodiment, the polypeptides are derived from a mammalian species for example human polypeptides.

Also disclosed are heterologous polypeptides that serve as reporter polypeptides comprising a detectable moiety.

The detectable moiety can be a member of a binding pair, which is identifiable via its interaction with an additional member of the binding pair and a label which is directly visualized. In one example, the member of the binding pair is an antigen which is identified by a corresponding labeled antibody. In one example, the label is a fluorescent protein or an enzyme producing a colorimetric reaction. Exemplary detectable moieties include, but are not limited to green fluorescent protein (Genbank Accession No. AAL33912), alkaline phosphatase (Genbank Accession No. AAK73766), peroxidase (Genbank Accession No. NP_568674), histidine tag (Genbank Accession No. AAK09208), Myc tag (Genbank Accession No. AF329457), biotin ligase tag (Genbank Accession No. NP_561589), orange fluorescent protein (Genbank Accession No. AAL33917), beta galactosidase (Genbank Accession No. NM_125776), Fluorescein isothiocyanate (Genbank Accession No. AAF22695) and strepavidin (Genbank Accession No. S11540).

Additional detectable moieties include products of bacterial luciferase genes, e.g., the luciferase genes encoded by Vibrio harveyi, Vibrio fischeri, and Xenorhabdus luminescens, the firefly luciferase gene FFlux, and the like.

Constructs, Vectors and Expression Systems:

The disclosed regulatory elements (and heterologous nucleic acid operatively linked thereto) can be incorporated into any suitable expression system. Recombinant expression is usefully accomplished using a vector (i.e. expression construct), such as a plasmid. The vector can include a promoter operably linked to DNA encoding the regulatory element (i.e. SEQ ID NOs: 1-44) and a heterologous nucleic acid sequence (e.g., encoding a protein). The vector can also include other elements required for transcription and translation.

As used herein, the term “vector” refers to a carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying transcription termination-regulated constructs can be produced. Such expression vectors include, for example, pET, pET3d, pCR2.1, pBAD, pUC, and yeast vectors. The vectors can be used, for example, in a variety of in vivo and in vitro situations.

Viral vectors include adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Retroviral vectors, which are described in Verma (1985), include Murine Maloney Leukemia virus, MMLV, and retroviruses that express the desirable properties of MMLV as a vector. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA.

Exemplary viral vectors are Adenovirus, Adeno-associated virus, Herpes virus, Vaccinia virus, Polio virus, AIDS virus, neuronal trophic virus, Sindbis and other RNA viruses, including these viruses with the HIV backbone. Also contemplated are any viral families which share the properties of these viruses which make them suitable for use as vectors. Exemplary retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature.

Viral vectors have higher transaction (ability to introduce genes) abilities than do most chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

The term “promoter” as used herein refers to a sequence or sequences of DNA that function when in a relatively fixed location in regard to the transcription start site. A “promoter” contains core elements required for basic interaction of RNA polymerase and transcription factors and can contain upstream elements and response elements. Exemplary promoters contemplated by the present invention include, but are not limited to polyoma, Simian Virus 40 (SV40), adenovirus, retroviruses, hepatitis-B virus and cytomegalovirus promoters. According to a particular embodiment, the promoter is a bacterial promoter.

The term “enhancer” as used herein refers to a sequence of DNA that functions at no fixed distance from the transcription start site and can be either 5′ (Laimins, 1981) or 3′ (Lusky et al., 1983) to the transcription unit. Furthermore, enhancers can be within an intron (Banerji et al., 1983) as well as within the coding sequence itself (Osborne et al., 1984). They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression. Examples of enhancers contemplated by the present invention include the SV40 enhancer on the late side of the replication origin (bp 100-270), the cytomegalovirus early promoter enhancer, the polyoma enhancer on the late side of the replication origin, and adenovirus enhancers.

The promotor and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al., Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

The vectors can include nucleic acid sequence encoding a marker product. This marker product is used to determine if the gene has been delivered to the cell and once delivered is being expressed. Preferred marker genes are the E. Coli lacZ gene which encodes beta-galactosidase and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO ^{DHFR}-cells and mouse ^{LTK}-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

Exemplary host systems contemplated by the present invention include both prokaryotic and eukaryotic cells. These include, but are not limited to bacterial cells (e.g., E. coli), fungal cells (e.g., S. cerevisiae cells), plant cells (e.g., tobacco), insect cells (lepidopteran cells) and other mammalian cells (Chinese Hamster Ovary cells) and human cells.

Since the regulator elements uncovered by the present inventors are triggered by ligands, such ligands may be used to control expression of the heterologous nucleic acids.

Thus, according to another aspect of the present invention there is provided a method of controlling expression of a gene product comprising contacting a bacteria with a ligand of a ligand responsive element, wherein the bacteria comprises a nucleic acid sequence encoding the gene product, the nucleic acid sequence being operatively linked to:

(i) said ligand responsive element, wherein said ligand responsive element comprises a sequence as set forth in SEQ ID NOs: 1-44; and

(ii) a promoter, thereby controlling expression of the gene product, thereby controlling expression of the gene product.

Ligands of the ligand responsive element have been described herein above. According to this aspect of the present invention, the ligand is capable of penetrating the cell.

In one embodiment, presence of the ligand (beyond a threshold level) increases the ratio of premature termination of the gene product:mature termination of the gene product. In another embodiment, presence of the ligand (beyond a threshold level) increases the ratio of premature termination of the gene product:mature termination of the gene product.

The ligand may be added or removed from the system according to the desired level of expression of the gene.

In one embodiment, removal of the ligand may be effected using an aptamer comprising the ligand responsive element. The aptamer would serve as a competitive inhibitor of the ligand.

Thus, according to another aspect of the present invention there is provided an isolated RNA (i.e. aptamer) comprising a nucleic acid sequence as set forth in SEQ ID NOs: 45-88, or a DNA encoding same, wherein the RNA or DNA is no longer than 350 nucleotides in length.

In this aspect of the present invention the aptamer is not operatively linked to a signal generating moiety or a sequence encoding a gene product.

According to a particular embodiment, the RNA or DNA encoding the aptamer of this aspect of the present invention is no longer than 450 nucleotides, 400 nucleotides, no longer than 375 nucleotides, no longer than 350 nucleotides, no longer than 325 nucleotides, no longer than 300 nucleotides, no longer than 275 nucleotides, no longer than 250 nucleotides, no longer than 225 nucleotides, no longer than 200 nucleotides, no longer than 190 nucleotides, no longer than 180 nucleotides, no longer than 170 nucleotides, no longer than 160 nucleotides, no longer than 150 nucleotides, no longer than 140 nucleotides, no longer than 130 nucleotides, no longer than 120 nucleotides, no longer than 110 nucleotides, no longer than 100 nucleotides, no longer than 90 nucleotides, no longer than 80 nucleotides, no longer than 70 nucleotides, no longer than 60 nucleotides, or even no longer than 50 nucleotides.

In another embodiment, removal of the ligand is effected by addition of an analog of the ligand (i.e. that competes for the trigger molecule) that does not activate the regulatory element.

As well as useful for controlling expression of a polypeptide in a host system, the regulatory elements disclosed herein may be used for sensing the presence of a ligand.

Thus, according to still another aspect of the present invention there is provided a method of detecting a ligand in a sample comprising:

(a) culturing bacteria in a medium comprising the sample; and

(b) measuring a level of expression of the reporter polypeptide, wherein a change in the level of expression of the reporter polypeptide as compared to the level of the reporter polypeptide measured when the bacteria are cultured in a medium devoid of the ligand, is indicative that the sample comprises the ligand.

According to this aspect of the present invention the bacteria are genetically modified to express a polynucleotide encoding the regulatory elements disclosed herein (i.e. SEQ ID NOs: 1-44) operatively linked to a reporter polypeptide.

The ligand of this aspect of the present invention is preferably one that traverses a cell membrane and can be taken up into the cell.

According to a particular embodiment, the ligand is an antibiotic, as described herein above. Particular examples of antibiotics which may be detected include, but are not limited to lincomycin, erythromycin, chloramphenicol, kanamycin, ofloxacin, ampicilin, tylosin and bacitracin.

According to a particular embodiment, when detecting an antibiotic, the bacteria are genetically modified to express the regulatory element comprising the sequence as set forth in SEQ ID NOs: 23, 27, 38 and 41. More specifically, for detection of lincomycin only, the regulatory element comprising the sequence as set forth in SEQ ID NOs: 23 or 41 should be used.

Samples which may be analyzed include biological samples (including body fluids such as blood, serum, saliva etc.), food samples, including dairy products such as milk, yoghurts, cream etc. and environmental samples including water, soil etc.

Reporter polypeptides according to this aspect of the present invention are described herein above.

The bacteria is typically cultured under conditions (e.g., length of time, temperature, pH conditions etc.) which allow for expression of the reporter polypeptide.

Methods of measuring the reporter polypeptide are known to those of skill in the art and the selection of the particular method is dependent upon the detectable moiety which is used in the system. For example, the reporter polypeptide may be detected using standard techniques (e.g., radioimmunoassay, radio-labeling, immunoassay, assay for enzymatic activity, absorbance, fluorescence, luminescence, and Western blot). More preferably, the level of the reporter protein is easily quantifiable using standard techniques even at low levels. Useful reporter proteins include luciferases, green fluorescent proteins and their derivatives, such as firefly luciferase (FL) from Photinus pyralis, and Renilla luciferase (RL) from Renilla reniformis.

According to a particular embodiment there is an increase (e.g., by at least 1.5 fold, 2 fold, 5 fold or even 10 fold or more) in the level of expression of the reporter polypeptide in the presence of the ligand as compared to the level of the reporter polypeptide measured when the bacteria are cultured in a medium devoid of the ligand.

According to another embodiment there is a decrease (e.g., by at least 1.5 fold, 2 fold, 5 fold or even 10 fold or more) in the level of expression of the reporter polypeptide in the presence of the ligand as compared to the level of the reporter polypeptide measured when the bacteria are cultured in a medium devoid of the ligand.

The genetically modified bacteria may also be used to screen for agents comprising a transcription terminating activity. If such agents are detected, they can be analyzed for additional activity such as antimicrobial activity.

In another aspect, there is provided a method of determining whether an agent is a transcription terminator comprising:

(a) culturing bacteria (which are genetically modified to express a polynucleotide encoding the regulatory elements disclosed herein (i.e. SEQ ID NOs: 1-44) operatively linked to a reporter polypeptide) in a medium comprising said agent; and

(b) measuring the level of expression of said reporter polypeptide, wherein a change in said level of expression of said reporter polypeptide as compared to the level of said reporter polypeptide measured when the bacteria are cultured in a medium devoid of said agent is indicative that the agent is a transcription terminator.

The phrase “antimicrobial activity” as used herein, refers to an ability to suppress, control, inhibit or kill microorganisms, such as bacteria, archaea and fungi. Thus for example the antimicrobial activity may comprise bactericidal or bacteriostatic activity, or both.

For any of the applications described herein above, the present invention also contemplates use of RNA aptamers which comprise any of the regulatory sequences as set forth in SEQ ID NOs: 45-89, operatively linked to a signal generating moiety.

Such RNA aptamers may be referred to as biosensor riboswitches. These are engineered riboswitches that produce a detectable signal in the presence of their cognate trigger molecule (i.e. ligand). Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch.

Conformation dependent labels refer to all labels that produce a change in fluorescence intensity or wavelength based on a change in the form or conformation of the molecule or compound (such as a riboswitch) with which the label is associated. Examples of conformation dependent labels used in the context of probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes. Such labels, and, in particular, the principles of their function, can be adapted for use with riboswitches. Several types of conformation dependent labels are reviewed in Schweitzer and Kingsmore, Curr. Opin. Biotech. 12:21-27 (2001).

Stem quenched labels, a form of conformation dependent labels, are fluorescent labels positioned on a nucleic acid such that when a stem structure forms a quenching moiety is brought into proximity such that fluorescence from the label is quenched. When the stem is disrupted (such as when a riboswitch containing the label is activated), the quenching moiety is no longer in proximity to the fluorescent label and fluorescence increases. Examples of this effect can be found in molecular beacons, fluorescent triplex oligos, triplex molecular beacons, triplex FRET probes, and QPNA probes, the operational principles of which can be adapted for use with riboswitches.

Stem activated labels, a form of conformation dependent labels, are labels or pairs of labels where fluorescence is increased or altered by formation of a stem structure. Stem activated labels can include an acceptor fluorescent label and a donor moiety such that, when the acceptor and donor are in proximity (when the nucleic acid strands containing the labels form a stem structure), fluorescence resonance energy transfer from the donor to the acceptor causes the acceptor to fluoresce. Stem activated labels are typically pairs of labels positioned on nucleic acid molecules (such as riboswitches) such that the acceptor and donor are brought into proximity when a stem structure is formed in the nucleic acid molecule. If the donor moiety of a stem activated label is itself a fluorescent label, it can release energy as fluorescence (typically at a different wavelength than the fluorescence of the acceptor) when not in proximity to an acceptor (that is, when a stem structure is not formed). When the stem structure forms, the overall effect would then be a reduction of donor fluorescence and an increase in acceptor fluorescence. FRET probes are an example of the use of stem activated labels, the operational principles of which can be adapted for use with riboswitches.

Sequence Similarities:

In general, for any sequence provided herein, the present invention also contemplates variants thereof.

In general, variants of riboswitches, aptamers, expression platforms, genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to a stated sequence or a native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al., Proc. Natl. Acad. Sci. USA 86:7706-7710, 1989, Jaeger et al., Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity.

For example, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

Nucleic Acids:

Numerous molecules disclosed herein are nucleic acid based, including, for example, riboswitches, aptamers, and nucleic acids that encode riboswitches and aptamers. The disclosed nucleic acids can be made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if a nucleic acid molecule is introduced into a cell or cell environment through for example exogenous delivery, it is advantageous that the nucleic acid molecule be made up of nucleotide analogs that reduce the degradation of the nucleic acid molecule in the cellular environment.

So long as their relevant function is maintained, riboswitches, aptamers, expression platforms and any other oligonucleotides and nucleic acids can be made up of or include modified nucleotides (nucleotide analogs). Many modified nucleotides are known and can be used in oligonucleotides and nucleic acids. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and 0-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10, alkyl or C2 to C10 alkenyl and alkynyl. 2′ sugar modifications also include but are not limited to —O[(CH.sub.2)nO]mCH.sub.3, —O(CH.sub.2)nOCH.sub.3, —O(CH.sub.2)nNH.sub.2, —O(CH.sub.2) nCH.sub.3, —O(CH.sub.2)n-ONH.sub.2, and O(CH.sub.2)nON[(CH.sub.2)nCH.sub.3)].sub.2, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH.sub.3, OCN, Cl, Br, CN, CF.sub.3, OCF.sub.3, SOCH.sub.3, SO.sub.2 CH.sub.3, ONO.sub.2, NO.sub.2, N.sub.3, NH.sub.2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH.sub.2 and S. Nucleotide sugar analogs can also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but can also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Oligonucleotides and nucleic acids can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. Such oligonucleotides and nucleic acids can be referred to as chimeric oligonucleotides and chimeric nucleic acids.

Solid Supports:

Solid supports are solid-state substrates or supports onto which the nucleic acid molecules of the present invention may be associated. The nucleic acids may be associated directly or indirectly. Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

In one embodiment, the solid support is an array which comprises a plurality of nucleic acids of the present invention immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al., (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al., (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Methods

Oligonucleotides, Wild-Type Bacterial Strains and Culture Conditions:

All oligonucleotides used in this study were purchased from Sigma or Integrated DNA Technologies (IDT) (Table 1).

TABLE 1

| | | |
|---|---|---|
| Term-seq RNA | NN-12mer_index-NNNN- | 5′ phosphorylated, 3′ amino |
| 3′ ligation | AGATCGGAAGAGCGTCGTGT | blocked and HPLC grade, |
| adapter | SEQ ID NO: 90 | N = random |
| Term-seq | TCTACACTCTTTCCCTACACGACGCT | Standard desalted |
| Reverse | CTTC | |
| transcription | SEQ ID NO: 91 | |
| primer | | |
| Term-seq | NNAGATCGGAAGAGCACACGTCTGA | 5′ phosphorylated, 3′ amino |
| cDNA 3′ adapter | ACTCCAGTCAC | blocked and HPLC grade, |
| | SEQ ID NO: 92 | N = random |
| Term-seq PCR | AATGATACGGCGACCACCGAGATCT | Standard desalted |
| forward | ACACTCTTTCCCTACACGACGCTCT | |
| | SEQ ID NO: 93 | |
| Term-seq PCR | CAAGCAGAAGACGGCATACGAGAT- | Standard desalted |
| reverse 1 | 8mer_index-GTGACTGGAGTTCAGAC | |
| | SEQ ID NO: 94 | |
| gBlock- | contains region 955141-955855 fused to | Mutations in L. |
| ATG > ACG | region 955857-956521, with a T > C | monocytogenes |
| | conversion at base 955856 | |
| gBlock-delta- | contains region 955201-955864 fused to | Mutations in L. |
| Anti-Anti- | region 955873-956580 | monocytogenes |
| terminator | | |
| gBlock-delta- | contains region 955201-955926 fused to | Mutations in L. |
| Anti-terminator | region 955935-956580 | monocytogenes |
| gBlock-Up | GTCAATACGACTCACTATAGGG SEQ | Mutations in L. |
| | ID NO: 95 | monocytogenes |
| gBlock-Down | CAAAAGCTGGTACCGGGCC SEQ ID | Mutations in L. |
| | NO: 96 | monocytogenes |
| Imo0919 | ATCAACCCGGGATCATTTTAACGACA | Mutations in L. |
| upstream | AACCGAGATG SEQ ID NO: 97 | monocytogenes |
| homology-F | | |
| Imo0919 | TTTTATTTAGCTTGAATAAAAAGACA | Mutations in L. |
| upstream | ACAGCCGTGTCGTTTGAAATACAC | monocytogenes |
| homology-R | SEQ ID NO: 98 | |
| Imo0919 | GTGTATTTCAAACGACACGGCTGTTG | Mutations in L. |
| downstream | TCTTTTTATTCAAGCTAAATAAAA | monocytogenes |
| homology-F | SEQ ID NO: 99 | |
| Imo0919 | ATCAAGGATCCCCGCCAGCAAGCGC | Mutations in L. |
| downstream | TATATTT SEQ ID NO: 100 | monocytogenes |
| homology R | | |

Bacillus subtilis str. 168, Listeria monocytogenes EGDe, and Enterococcus faecalis ATCC 29212 were cultured under aerobic conditions at 37° C. with shaking in either LB (10 g/L tryptone, 5 g/L yeast extract 5 g/L NaCl), TB (12 g/L tryptone, 24 g/L yeast extract, 0.4% glycerol, 2.2 g/L KH₂PO₄ and 9.4 g/L K₂HPO₄), Brain Heart Infusion (BHI) broth (Difco), or M9 minimal media (0.5% w/v glucose, 2 g/L [NH4]₂SO₄, 18.3 g/L K2 HPO₄.3H₂O, 6 g/L KH2PO₄, 1 g/L sodium citrate, 0.2 g/L MgSO₄.7H₂O, 5 μM MnCl₂, and 5 μM CaCl₂, tryptophan (Sigma) 50 μg/mL).

Lysine Responsive Regulation:

B. subtilis was grown overnight (O.N.) in LB and then diluted 1:200 into 150 ml of M9 media supplemented with lysine and methionine (50 μg/mL each). Bacteria were grown to OD₆₀₀=0.9-1.0, washed, and then resuspended to an OD₆₀₀=0.3 in 3 ml of M9 media, containing the following combinations of amino acids at a final concentration of 50 μg/mL: lysine and methionine (lys+met+), methionine only (lys−met+), or lysine only (lys+met−). Cells were incubated for 2 h and collected by centrifugation (4000 rpm, 5 min, and 4° C.) followed by flash freezing. Samples were stored in −80° C. until RNA extraction.

Antibiotics Responsive Regulators:

A sublethal concentration for the antibiotics lincomycin, erythromycin, chloramphenicol, kanamycin, ofloxacin, ampicilin and bacitracin (Sigma) was determined for each of the organisms used in this study as follows. Bacterial cultures were propagated in LB or BHI O.N. in triplicates and diluted 1:200 into fresh media. Cultures were grown to early exponential phase (OD₆₀₀=0.1-0.2) and then supplemented with serially diluted antibiotics stocks. The growth rate was dynamically monitored in intervals of 10-15 min using a 96 well plate format OD reader (Infinite M200 Tecan) for a period of at least 4 hours. The highest antibiotics concentration that did not cause growth-rate inhibition as compared to the no-antibiotics control was chosen as the sublethal dosage (Table 2, herein below). To identify antibiotic-responsive regulators, bacteria were grown in LB or BHI in triplicates as described above and, upon reaching early exponential phase, 5 ml cultures were independently exposed for 15 minutes to the sublethal concentration of each antibiotic as determined above. Bacteria were then collected by centrifugation, flash frozen and stored in −80° C. until RNA extraction.

TABLE 2

| Antibiotic (μg/ml) | B. subtilis | L. monocytogenes | E. faecalis |
|---|---|---|---|
| Lincomycin | 0.5 | 0.25 | 2 |
| Chloramphenicol | 0.2 | 0.4 | 0.4 |
| Erythromycin | 0.015625 | 0.03125 | 0.5 |
| Kanamycin | 0.5 | 0.5 | 16 |
| ciprofloxacin | 0.0625 | 2 | 1 |
| Ampicilin | 0.0625 | 1 | 2 |
| Bacitracin | 0.125 | 8 | 1 |

RNA Isolation:

Frozen bacterial pellets were lysed using the Fastprep homogenizer (MP Biomedicals) and RNA was extracted with the FastRNA PRO™ blue kit (MP Biomedicals, 116025050) according to the manufacturer's instructions. RNA levels and integrity were determined by Qubit® RNA BR Assay Kit (Life technologies, Q10210) and Tapestation (Agilent, 5067-5576), respectively. All RNA samples were treated with TURBO™ DNase (Life technologies, AM2238).

Constructions of Plasmids and Strains:

For mutant generation with pMAD-based plasmids⁵⁹, ˜600 nt regions of complementarity both upstream and downstream of a targeted region were either ordered as gBlocks (IDT) and amplified with gBlock-Up and Down oligonucleotides (Table 1) complimentary to uniform flanks on each gBlock corresponding to the 40 nts on either side of the pMAD multi-cloning site, or PCR amplified with Phusion High fidelity polymerase and reagents (Finnzymes, F-553) using genomic DNA as a template and then joined by a second splice overlap extension PCR reaction using the first two PCR products as template to generate a Upstream-Downstream (UD) PCR product (Table 1, lmo0919 deletion). PCR products were subsequently purified with QIAquick PCR purification columns (Qiagen, 28104), digested with the SalI and XmaI restriction enzymes (NEB), purified again as before, and ligated into SalI/XmaI digested pMAD plasmid for 1 hr at 25° C. with T4 DNA ligase (NEB, M0202S). 2 μl of each ligation were transformed into chemically competent E. coli Top10 (Invitrogen, C404003) cells according to the manufacturer's instructions. Transformants were screened by PCR and Sanger sequencing for the presence of the appropriate insert. Electrocompetent L. monocytogenes strains were transformed with the respective plasmid and mutagenesis carried out as described previously⁵⁹. Briefly, after transformation and plating onto selective BHI, 5 ug/ml erythromycin (Em) 80 ug/ml 5-bromo-4-chloro-3-indolyl-β-D-galactopyranoside (X-gal) plates, bacteria were grown at 30° C. for two days. A single blue colony was picked and transferred to liquid BHI broth and grown for an additional 6 hrs at 30° C. The colony was then diluted 1:1000 into 10 ml of BHI-Em and grown O.N. at 42° C., which prevents pMAD replication in the cytosol owing to a temperature sensitive ori. Serial dilutions were plated onto BHI-X-gal-Em plates and grown for two days at 42° C., and the process was reiterated several times until white colonies, in which the plasmid integrated into the genome, were recovered. Colonies were screened by colony PCR, and mutants were confirmed by Sanger sequencing.

Minimal Inhibitory Concentration (MIC) Determination:

The MIC was determined in broth culturing conditions in the presence of serially diluted antibiotic concentrations. Briefly, the bacterial strains were grown O.N. at 37° C. in BHI agarose plates and 1-3 single colonies were collected into 1 ml of BHI broth. The OD₆₀₀ was adjusted to 0.01 and then diluted 1:10 into a 96-well plate containing a final volume of 200 μl BHI supplemented with two-fold serial dilutions of lincomycin, erythromycin and chloramphenicol. The samples were grown over two days at 37° C. without shaking and the MIC was determined as the lowest antibiotic concentration to fully inhibit growth.

Term-Seq Library Preparation:

DNAse treated RNA (1-5 μg) was subjected to a 3′ end specific ligation by mixing 50 RNA solution with 1 μl of a 150 μM DNA adapter solution (Table 1), 2.5 μl 10×T4 RNA ligase 1 buffer, 2.5 μl 10 mM ATP, 2 μl DMSO, 9.5 μl 50% PEG8000 and 2.50 T4 RNA ligase 1 enzyme (NEB, M0204). The reaction was incubated for 2.5 h at 23° C. and then cleaned by adding 2.2× (55 μl) paramagnetic SPRI beads (Agencourt AMPure XP, Beckman Coulter), mixing well by pipetting and leaving the reaction-bead solution to rest at room temperature for 2 minutes. The supernatant was separated from the beads using a 96-well magnetic separator (Invitrogen). Beads were washed on magnet (beads securely attached) by discarding the solution and adding 120 μl 70% ethanol (EtOH), allowing an incubation period of 1 min. The cleanup stage was repeated and the beads were air dried for 5 min. The RNA was eluted in 5-10 μl H₂O. The RNA was fragmented with fragmentation buffer (Ambion) in 72° C. for 1.5 min. The fragmentation reaction was cleaned using SPRI beads 2.2× as described above, and eluted in 28 μl H₂O. Ribosomal RNA was depleted using the Ribo-Zero™ rRNA Removal Kit (epicenter, MRZB12424) or MICROBExpress™ (Life technologies, AM1905) according to the manufacturer's instructions. Depleted RNA was reverse transcribed by incubating 11 μl of RNA with 1 μl of 10 μM reverse transcription primer (Table 1), incubating at 65° C. for 5 min and immediately placing on ice for 2 min. 2 μl AffinityScript reverse transcriptase (Agilent, 600559), 2 μl 10× Affinity Script Buffer, 2 μl 100 mM DTT and 2 μl 10 mM dNTPs (Sigma, D7295) were added, and the reaction was incubated at 42° C. for 45 min and then terminated by incubation at 75° C. for 15 min. To degrade the RNA template, 1 μl of RNase H (NEB, M0297L) was added and the reaction was incubated for an additional 30 min at 37° C. The reaction was cleaned by using SPRI beads at a 2.2× ratio (46p1) and eluted in 5.5 μl H₂O. 5p1 of the resulting cDNA was subjected to a second 3′ end ligation, as above, but using a cDNA specific ligation adapter (Table 1). The reaction was incubated at 23° C. for 4-8 h and then cleaned with SPRI beads at a 1.8× ratio (45 μl), eluting the cDNA in 23 μl H₂O. 22 μl of ligated cDNA solution was mixed with 1.5 μl of forward and reverse primers, at 25 μM each (Table 1) and 25 μl KAPA Hi-Fi PCR ready mix (KAPA Biosystems, KK2601). Library was amplified using the manufacturer's protocol with 16-18 amplification cycles. The final term-seq library was cleaned with SPRI beads at a 0.9× ratio (45 μl) and the concentration and size distribution were determined by Qubit® dsDNA BR Assay Kit (Life technologies, Q32850) and the dsDNA D1000 Tapestation kit (Agilent, 5067-5582), respectively.

RNA-Seq and 5′ End Sequencing:

For RNA-seq library preparation, 4 μg DNase treated RNA was fragmented in 20 μL reaction volume as described above and cleaned by adding 2.2× (50p1) SPRI beads and 30% v/v Isopropanol (30p1). The beads were washed with 120 μl 80% EtOH and then air dried as described above. The RNA was eluted in 26μl H₂O and ribosomal RNA was depleted as in term-seq. Strand specific RNA-seq was performed with the NEBNext® Ultra™ Directional RNA Library Prep Kit (NEB, E7420) with the following adjustments to the manufacturer's instructions: All cleaning steps were carried out with 2.2×SPRI beads and 30% v/v isopropanol combinations, the washing steps were performed with 450 μl 80% EtOH, and only one cleanup step was performed after the end repair step. The resulting libraries concentrations and sizes were evaluated as in term-seq. For 5′end sequencing, the RNA was divided into a Tobacco Acid Pyrophosphatase (TAP) treated and untreated (noTAP) reactions which were subsequently sequenced using a 5′ end specific library preparation protocol described in Wurtzel et al., (Ref 32). In B. subtilis, the 5′ end libraries were prepared with bacteria grown to early exponential phase in TB medium. For L. monocytogenes, 5′ end data was taken from Wurtzel et al., (Ref 32).

Deep-Sequencing, Read Mapping and Counting:

RNA-seq, 5′end and term-seq libraries were sequenced using the Illumina Miseq, Hiseq1500 or NextSeq500 platforms. Sequenced reads were demultiplexed and adapters were trimmed using Casava v1.8.2. Reads were mapped to the reference genomes (Gene annotation and sequences were downloaded from Genbank: AL009126, NC_003210, NC_004668 for Bacillus subtilis str. 168, Listeria monocytogenes EGD-e, Enterococcus faecalis V583, respectively) using NovoAlign (Novocraft) V3.02.02 with default parameters, discarding reads that were non-uniquely mapped as previously described in Wurtzel et al., (Ref 32). All downstream analyses were performed using custom written perl and R script.

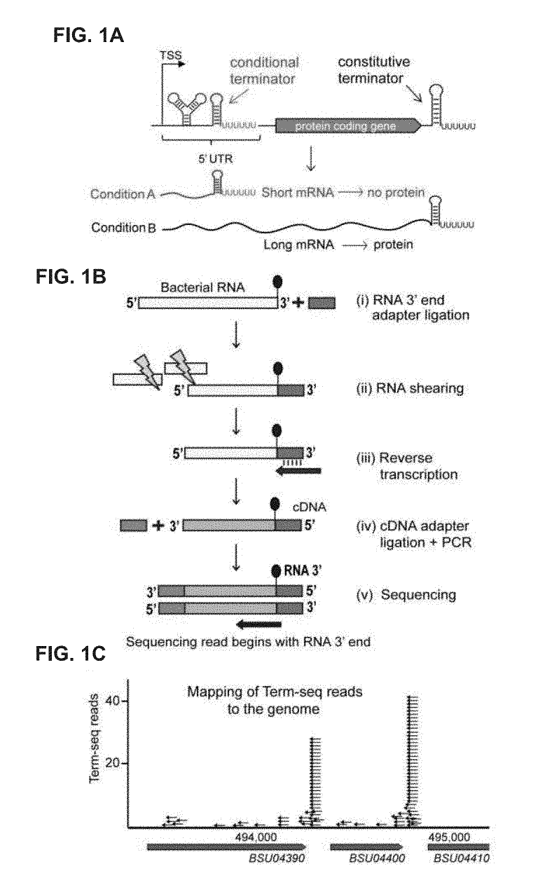

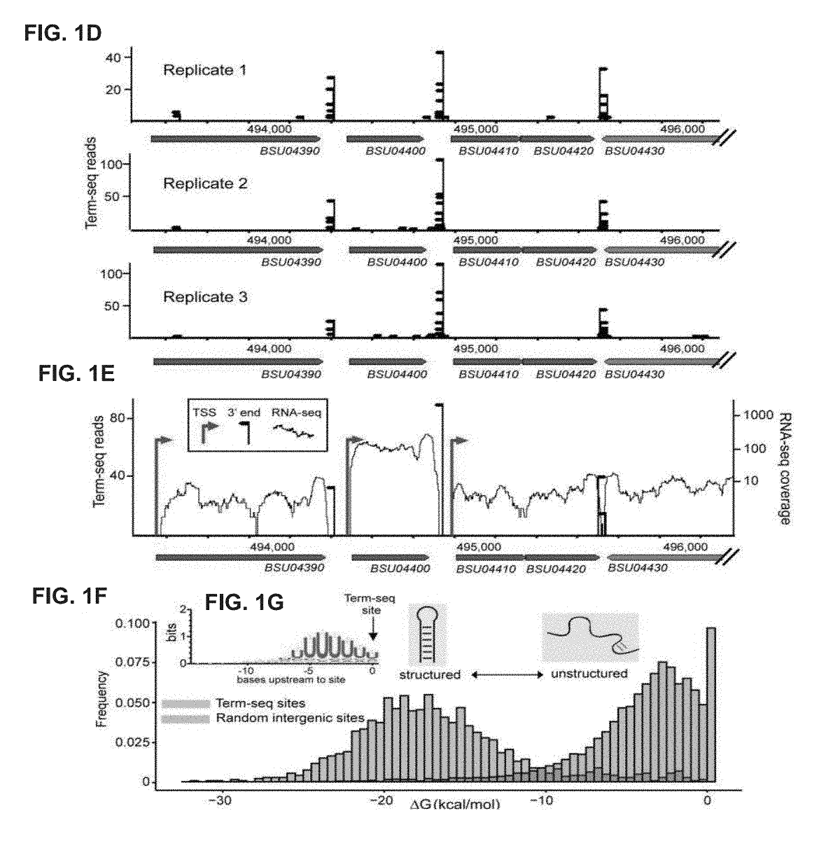

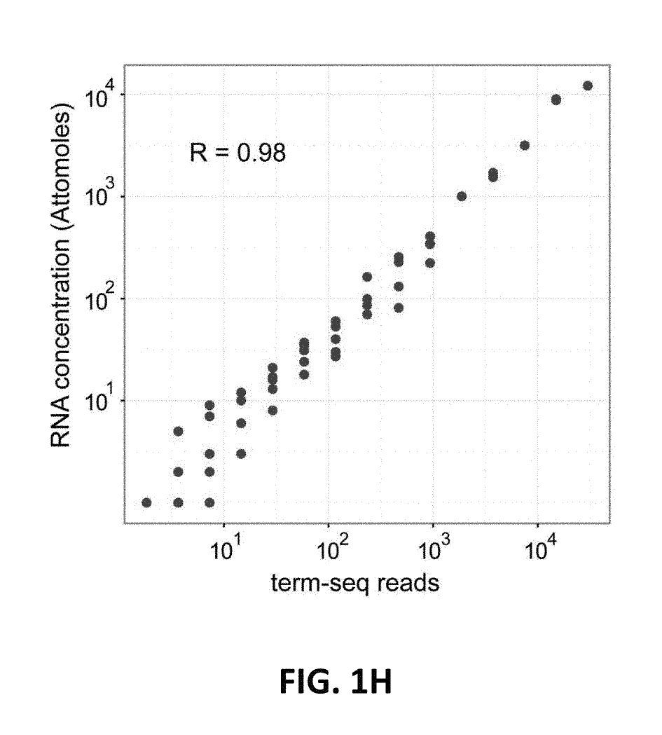

RNA-seq-mapped reads were used to generate genome-wide RNA-seq coverage maps. 5′end and term-seq positions were determined as the first nucleotide position of the mapped read. Total 5′end or term-seq coverage was calculated per nucleotide position in the genome. The data was visualized using a custom browser as described in Wurtzel et al., (Ref 32) (FIGS. 1A-5H).

TSSs were determined as in Wurtzel et al., (Ref 32). Briefly, the ratio between TAP-treated (TAP) and untreated (noTAP) was calculated for each genomic position covered by least 4 reads in the TAP condition. The maximal 5′UTR allowed was set to 450 nt and the TSS was chosen as the site with a TAP/noTAP ratio greater than 2 for B. subtilis and greater than 1 for L. monocytogenes and E. faecalis. In cases where multiple potential TSSs were available, the site with the highest coverage was chosen as the gene TSS.

Terminator Identification and Analysis:

For the assignment of terminators to genes, the downstream sequence of each gene (up to 150 nt, allowing up to 10 nt invasion to the next gene) was scanned for term-seq sites that were covered by a minimum of 4 reads in each of the three biological replicates. In case multiple sites were observed, the site with the highest coverage was selected as the terminator. For terminator sequence and structure analysis, the 40 nt upstream and 20 nt downstream sequences were collected for each terminator and folded in-silico via the RNAfold software using the standard parameters³³.

Discovery of Premature Termination:

For the discovery of premature termination, the 5′UTR (the beginning of which was defined by the TSS) of each gene was scanned for term-seq sites that were covered by a minimum of 2 reads in each of the three biological replicates. Since the average length of a terminator is approximately 20-25 nt⁶⁰, only 5′UTRs where the distance between the TSS and the term-seq site was at least 70 nt were considered. Candidate regulators that displayed high term-seq density also across the gene body were discarded as likely degraded transcripts. Due to specific regulator degradation/processing patterns, a handful of premature terminator sites were manually corrected to a nearby, less covered term-seq site, if that site presented a stem-loop and polyU signature. To differentiate between known and novel regulators, all candidate elements were compared against the Rfam database⁶¹ (Rfam 11.0 2012-07-19: AL009126.3, AL591824.1 and AE016830.1 for B. subtilis, L. monocytogenes and E. faecalis, respectively) and the literature. All identified candidate regulators were independently compared to the online Rfam db.

Transcriptional Readthrough Estimation Using RNA-Seq and Term-Seq:

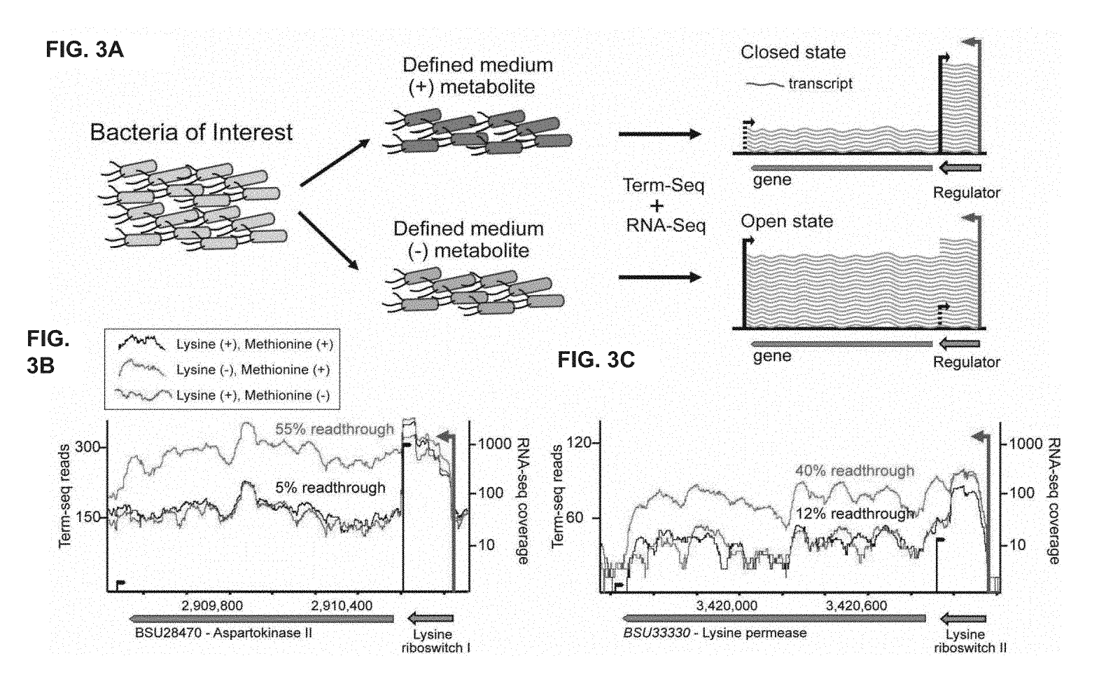

Term-seq average coverage across triplicates was calculated for the premature termination site and the full length gene termination site with a span of 10 nt surrounding each terminator, and the fraction of full length (gene) terminations out of all termination events was used to as a measure of the transcriptional readthrough (FIG. 3A). In cases where the regulator controlled the transcription of a multi-gene operon, which contained internal TSSs in addition to the primary one, RNA-seq was used to determine readthrough in the first gene (FIGS. 4C and 4F). RNA-seq coverage was used to measure the median read coverage over either the regulatory element or the gene, and the ratio between the two (gene-coverage divided by regulator-coverage) was used as an estimate for the short/long transcript ratio generated by regulator activity (FIG. 3A).

### Example 1

### Genome-Wide Mapping of RNA 3′ Termini Via Term-Seq

To identify regulated transcriptional termination events in an unbiased manner, the present inventors sought to map all RNA termini that are present in the cell at a given condition. As opposed to eukaryotic RNA, where the presence of the nearly universal polyA tail allows direct reverse transcription priming from the 3′ end, the absence of 3′ polyadenylation in bacterial mRNAs makes 3′ ends mapping less trivial. An RNA-seq protocol (denoted here ‘term-seq’) was developed that directly sequences exposed RNA 3′ ends in bacteria, yielding a quantitative genome-wide map of RNA termini. In this method, an Illumina adaptor is ligated to the RNA 3′ ends prior to reverse transcription, so that the first base of each resulting sequencing read maps to the last base of the RNA molecule, thus determining the exact position of the RNA 3′ end (FIG. 1B, Methods).

Applying term-seq to a large set of synthetic transcripts mixed in various, predetermined concentrations verified that term-seq accurately reconstitutes the exact 3′ end termini in a highly quantitative manner (FIG. 1H).

To assess the capacity of this protocol to map bona fide bacterial RNA termini, term-seq was applied on Bacillus subtilis, an organism in which >4% of all genes are predicted to be controlled by riboswitches and other termination-based regulators²⁹. RNA was extracted from exponentially growing cells in biological triplicates, and reads resulting from term-seq were mapped to the genome (FIG. 1C; Methods). Examining the genomic positions of the mapped reads showed that 69.5% (+/−1.8%) of all reads mapped to non-protein-coding regions, supporting that these reads largely represent RNA termini. To avoid reads that represent degraded RNA, positions that were independently reproduced in all three biological replicates, and that were supported by at least 4 reads in each of the replicates were considered (FIG. 1D; Methods). This filtering resulted in 84.4% (+/−0.7%) of the reads mapping to non-coding positions, representing >50 fold enrichment over what would have been expected by chance based on the coding: non-coding composition of the B. subtilis genome (p=0, binomial exact test). These results imply that the sites mapped via term-seq represent native RNA termini existing in the cell.

The 3′ end transcriptome was analyzed by counting the number of term-seq reads that mapped to each genomic position. To examine to what extent the sites defined by this approach represent transcription termination events, each gene was associated with its respective term-seq-inferred termination position in the downstream intergenic region (Methods). In cases where multiple nearby sites were possible for a given gene, the one supported by the highest number of reads was selected (FIG. 1E; Methods). This analysis yielded a collection of 1489 predicted termination sites which, considering polycistronic transcripts, explains termination for 55% (2300/4200) of the genes in the B. subtilis genome.

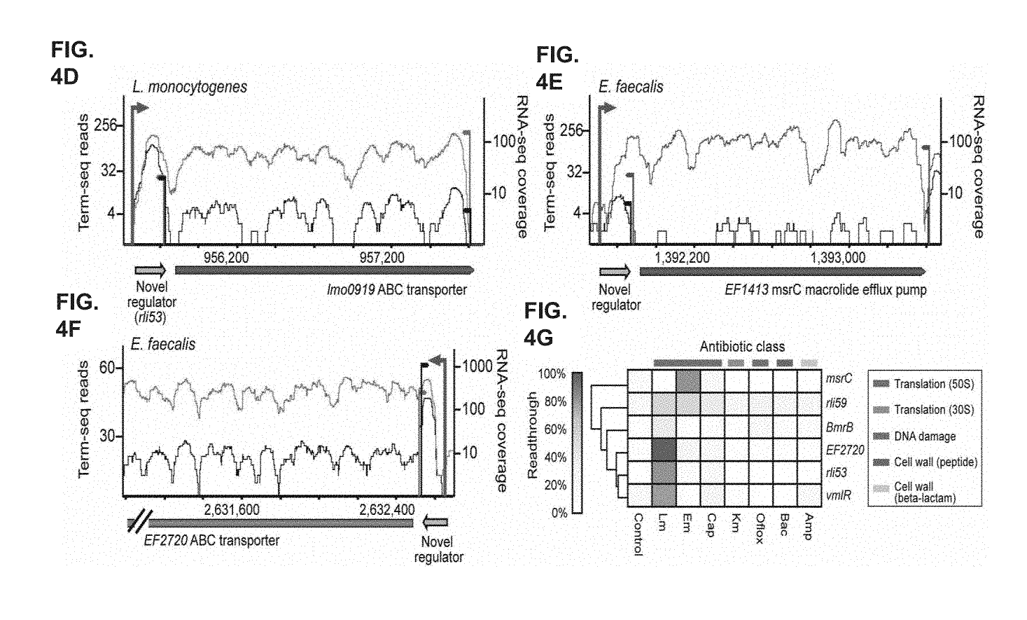

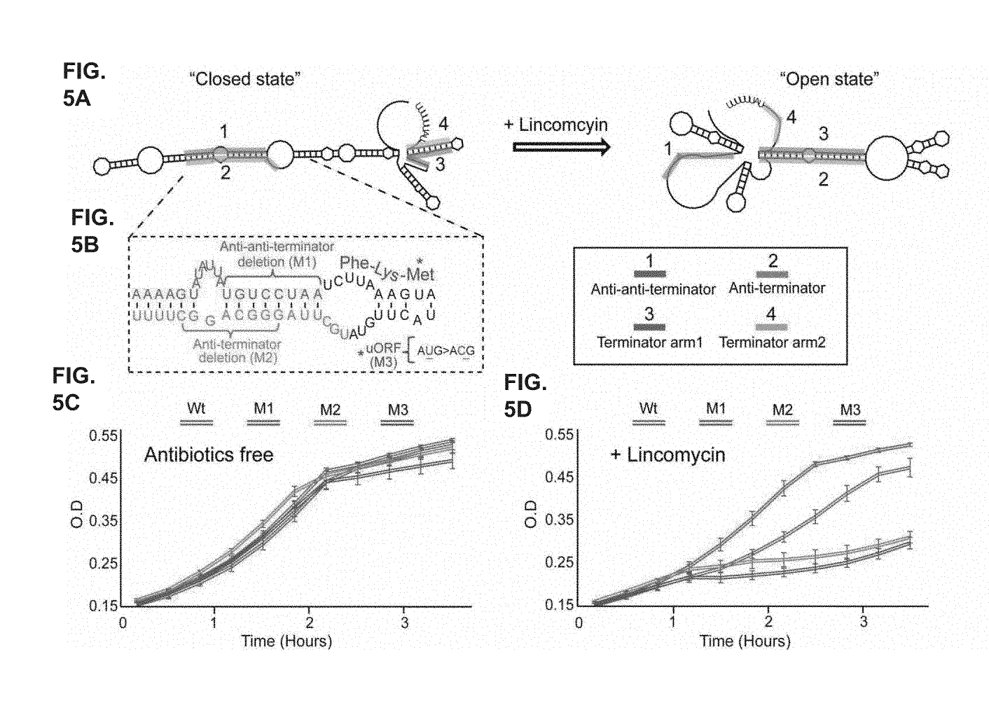

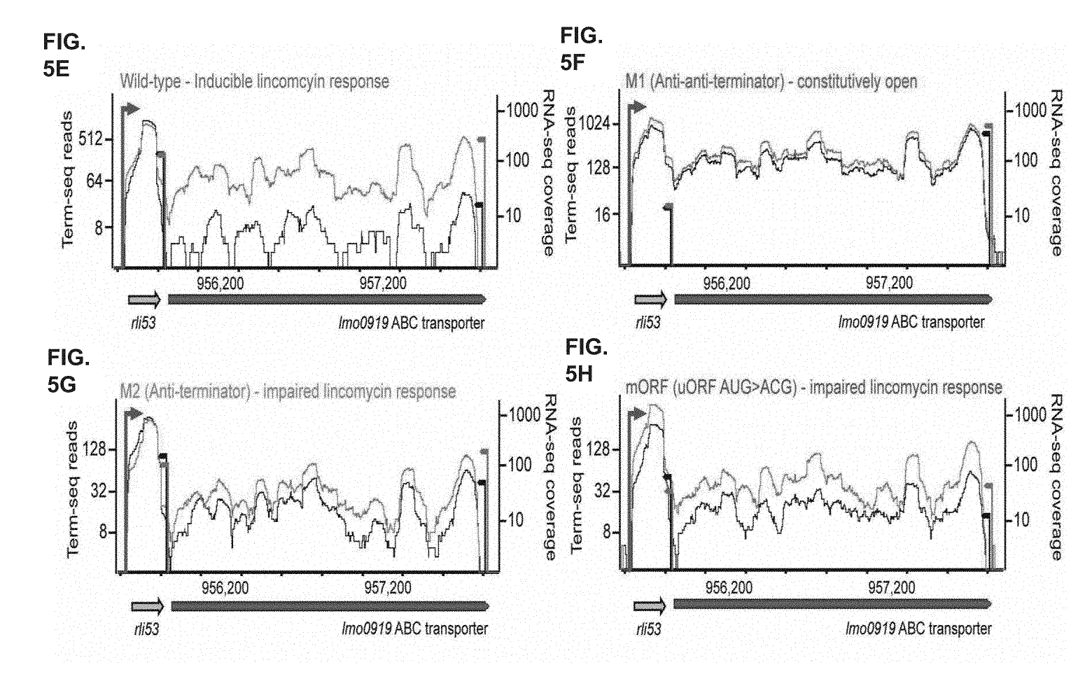

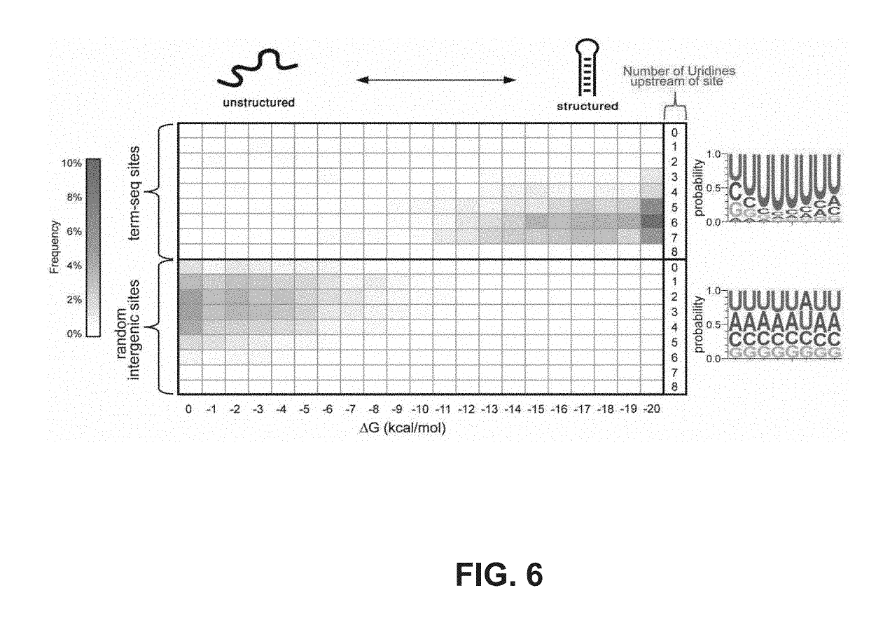

Bacterial Intrinsic (rho-independent) transcriptional terminators are characterized by a stem/loop structure followed by a uridine-rich tail³⁰. Indeed, the predicted structures and uridine content of the RNA termini defined by term-seq were strongly indicative of them being bona fide transcriptional terminators (FIGS. 1F-G; FIG. 6). For 94% (1399/1489) of the sites predicted as terminators, there was a clear stem/loop structure preceding the site, and 91% (1355/1489) of them had at least 4 uridine residues in the eight bases immediately upstream to the termination position (FIG. 6). These results demonstrate the ability of term-seq to experimentally map RNA termini to the single-base resolution across the bacterial genome.

### Example 2

### A Platform for the Discovery of Genes Regulated by Conditional Termination

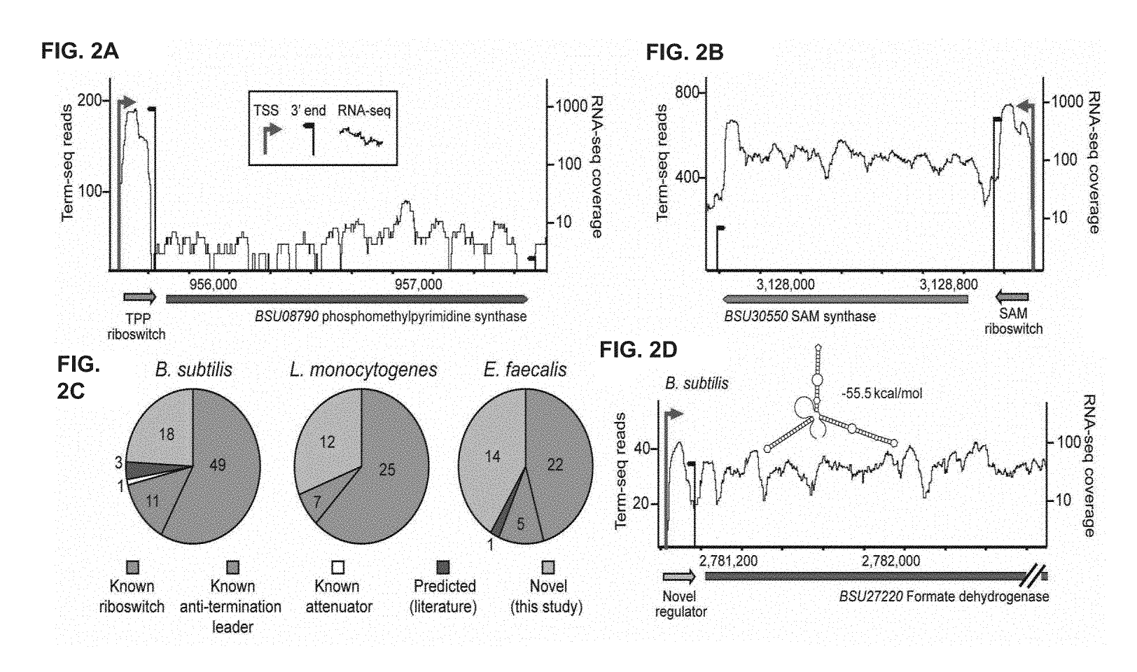

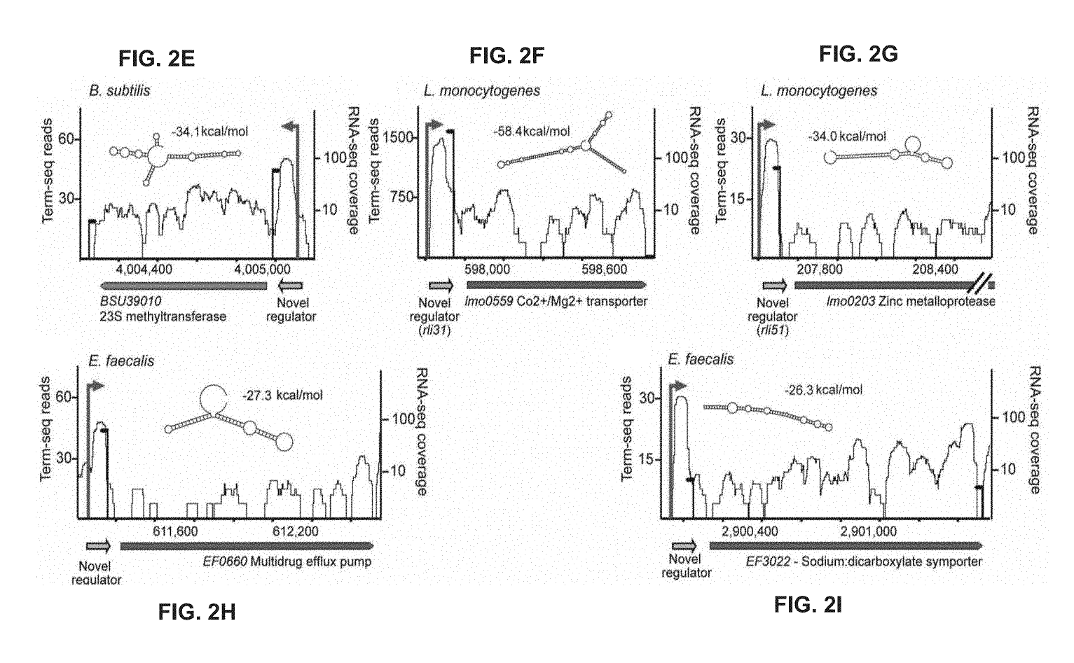

The present inventors next sought to examine whether term-seq can be used to identify regulated, premature termination events. It was reasoned that genes that are transcriptionally regulated by riboswitches and attenuators will present a premature termination site within their 5′UTR, downstream to the transcription start site (TSS). TSSs were mapped across the B. subtilis genome using a genome-wide 5′ end sequencing protocol^{31,32} (Methods), which included a standard RNA-seq coverage data to gain a comprehensive view of the B. subtilis transcriptome (FIG. 1E; Methods). Examining known B. subtilis riboswitches showed a clear pattern of reproducible premature termination sites at the 5′ UTR, supporting the hypothesis that such sites can be indicative of riboswitch activity (FIGS. 2A-B).

To assess the sensitivity and specificity of this method in exposing genes controlled by regulated termination, the present inventors searched for all genes that contained a reproducible term-seq site within their 5′UTRs (Methods). The B. subtilis genome contains 53 transcriptional units (TUs) regulated by known riboswitches, and the present approach recovered 49 (92%) of these (FIG. 2C; Methods). Four known riboswitch-regulated genes have escaped detection, either because they were under the control of multiple consecutive riboswitches, lacked a mapped TSS, or due to an annotation error that placed the riboswitch within a misannotated ORF. These results therefore show a high sensitivity for the present method in mapping riboswitches in a genome-wide manner.

Overall, the search retrieved 82 candidate regulatory elements, of which 64 (78%) were mapped to previously reported elements (FIG. 2C). In addition to the 49 known riboswitches, the algorithm also recovered 11 cases of conditional termination known to be regulated by RNA-binding anti-termination proteins including TRAP³⁵, GlpP³⁶, PyrR³⁷, and PTS system proteins³⁸. One case of known attenuation was identified³⁹, as well as three elements (the rimP, Pan and vmlR leaders^{25,40,41}) previously predicted, but not validated, as being cis-regulatory elements in B. subtilis.

Arguably the most studied Gram-positive model organism, B. subtilis has one of the best annotated genomes in the bacterial domain. Nevertheless, the present data enabled the detection of 18 new elements predicted to regulate gene expression by premature termination, increasing the putative number of such elements in B. subtilis by more than 20% (FIG. 2C). These included predicted regulatory elements upstream of the formate dehydrogenase gene yrhE (FIG. 2D); the GMP synthase gene guaA; yfnI, a gene responsible for the biosynthesis of the polyglycerolphosphate moiety of lipoteichoic acid; and yxjB, a 23S rRNA (guanine⁷⁴⁸-N1)-methyltransferase predicted to confer resistance to macrolide antibiotics (FIG. 2E; Table 3, herein below). Many of these new candidate regulators encompass complex predicted RNA secondary structures (FIGS. 2D-E). The various functions encoded by the genes associated with the new regulators, and the lack of homology to any known riboswitch or other RNA elements in the RNA family database (Rfam⁴²), suggests that these regulatory elements may respond to novel ligands, anti-termination proteins or attenuation principles.

TABLE 3
Novel regulators in the B. subtilis genome discovered via term-seq

| | | | Transcription | Term-seq | Regulator | | |
|---|---|---|---|---|---|---|---|
| | | | start site | position | length | Putative | SEQ |
| Regulator | Regulated | | (TSS) | (premature | (TSS to | intrinsic | ID |
| name^{a} | gene | Gene annotation | position^{b} | terminator) | terminator) | terminator?^{c} | NO |
| TBR-BSU1 | BSU00010 | chromosomal replication initiator | 157 | 270 | 114 | yes | 1 |
| TBR-BSU2 | BSU00380 | methionyl-tRNA synthetase | 45536 | 45607 | 72 | yes | 2 |
| TBR-BSU3 | BSU02780 | lipoprotein involved in swarming | 300681 | 300522 | 160 | no | 3 |
| TBR-BSU4 | BSU04490 | putative ABC transporter | 502668 | 502883 | 216 | yes | 4 |
| TBR-BSU5 | BSU06360 | GMP synthetase | 692609 | 692701 | 93 | yes | 5 |
| TBR-BSU6 | BSU07260 | lipoteichoic acid synthase | 796130 | 796224 | 95 | yes | 6 |
| TBR-BSU7 | BSU09590 | putative membrane protein | 1035409 | 1035538 | 130 | yes | 7 |
| TBR-BSU8 | BSU16490 | ribosomal protein S2 | 1717822 | 1717919 | 98 | no | 8 |
| TBR-BSU9 | BSU18090 | subunit B of DNA topoisomerase IV | 1933177 | 1933398 | 222 | no | 9 |
| TBR-BSU10 | BSU18970 | putative NTPase with transmembrane helices | 2069168 | 2069076 | 93 | no | 10 |
| TBR-BSU11 | BSU24300 | exodeoxyribonuclease VII | 2528397 | 2528308 | 90 | yes | 11 |
| TBR-BSU12 | BSU26220 | conserved phage gene | 2691617 | 2691468 | 150 | yes | 12 |
| TBR-BSU13 | BSU27220 | putative formate dehydrogenase | 2781016 | 2781172 | 157 | yes | 13 |
| TBR-BSU14 | BSU29660 | ribosomal protein S4 (BS4) | 3035548 | 3035691 | 144 | no | 14 |
| TBR-BSU15 | BSU31130 | putative efflux transporter | 3193540 | 3193430 | 111 | yes | 15 |
| TBR-BSU16 | BSU32120 | hypothetical protein | 3302876 | 3302795 | 82 | yes | 16 |
| TBR-BSU17 | BSU39010 | putative 23S rRNA (guanine748-N1)- | 4005313 | 4005183 | 131 | yes | 17 |
| | | methyltransferase | | | | | |
| TBR-BSU18 | BSU40520 | putative integral membrane protein | 4166709 | 4166621 | 89 | yes | 18 |

^{a}Serial number of novel Termination-Based-Regulators (TBRs) discovered in this study in B. subtilis
^{b}TSSs were inferred from transcriptome-wide sequencing of RNA 5′ ends³² (Methods).
^{c}Indicates whether the term-seq position was preceded by a stem-loop-polyU signature, indicative of intrinsic terminators.

The present inventors further explored the discovery potential of the present approach by applying term-seq on two clinically important pathogens: Listeria monocytogenes (L. monocytogenes), an abundant food-borne pathogen that causes gastroenteritis and can lead to severe sepsis and meningitis in immunocompromised patients, and abortion in pregnant women⁴³, and Enterococcus faecalis (E. faecalis), a frequent causal agent of nosocomial endocarditis, bacteremia and meningitis⁴⁴. Similar to B. subtilis, in both pathogens term-seq detected most of the known riboswitches that function via regulated termination, as well as multiple predicted novel regulators (12 in L. monocytogenes and 14 in E. faecalis; FIGS. 2C, F-I; Tables 4-5).

TABLE 4
Novel regulators in the L. monocytogenes genome discovered via term-seq

| | | | Transcription | Term-seq | Regulator | | | |
|---|---|---|---|---|---|---|---|---|
| | | | start | position | length | Putative | Previously | SEQ |
| Regulator | Regulated | | site (TSS) | (premature | (TSS to | Intrinsic | identified as | ID |
| name^{a} | gene | Gene annotation | position^{b} | terminator) | terminator) | terminator? | expressed RNA^{d} | NO |
| TBR-lmo1 | lmo0203 | Zinc metalloproteinase | 207589 | 207712 | 124 | yes | rli51 (Ref 45) | 19 |
| TBR-lmo2 | lmo0517 | Phosphoglycerate mutase | 552417 | 552315 | 103 | yes | rli52 (Ref 45) | 20 |
| TBR-lmo3 | lmo0559 | Mg2+/Co2+ transporter | 597806 | 597990 | 185 | yes | rli31 (Ref 45) | 21 |
| TBR-lmo4 | lmo0897 | Sulfate permease | 932139 | 932221 | 83 | yes | | 22 |
| TBR-lmo5 | lmo0919 | ABC transporter ATPase | 955824 | 956030 | 207 | yes | rli53 (Ref 45) | 23 |
| TBR-lmo6 | lmo1252 | similar to B. subtilis YxkD protein | 1276834 | 1276713 | 122 | yes | rli41 (Ref 45) | 24 |
| TBR-lmo7 | lmo1573 | acetyl-CoA carboxylase subunit b | 1613451 | 1613359 | 93 | no | rli129 (Ref 32) | 25 |
| TBR-lmo8 | lmo1596 | 30S ribosomal protein S4 | 1638956 | 1639080 | 125 | no | | 26 |
| TBR-lmo9 | lmo1652 | ABC transporter ATPase | 1702543 | 1702330 | 214 | yes | rli59 (Ref 45) | 27 |
| TBR-lmo10 | lmo2187 | Protein of unknown function | 2275362 | 2275261 | 102 | yes | rli61 (Ref 45) | 28 |
| TBR-lmo11 | lmo2758 | inosine-5′-monophosphate | 2839573 | 2839453 | 121 | yes | rli113 (Ref 46) | 29 |
| | | dehydrogenase | | | | | | |
| TBR-lmo12 | lmo2760 | ABC transporter ATPase | 2842199 | 2841960 | 240 | yes | rliI (Ref 46) | 30 |

^{a}Serial number of novel Termination-Based-Regulators (TBRs) discovered in this study in L. monocytogenes
^{b}TSSs were inferred from transcriptome-wide sequencing of RNA 5′ ends³² (Methods).
^{c} Indicates whether the term-seq position was preceded by a stem-loop-polyU signature, indicative of intrinsic terminators.
^{d}Specifies whether a candidate regulatory element was previously identified as an expressed RNA.

TABLE 5
Novel regulators in the E. faecalis genome discovered via term-seq

| | | | Transcription | Term-seq | Regulator | | |
|---|---|---|---|---|---|---|---|
| | | | start site | position | length | Putative | SEQ |
| Regulator | Regulated | | (TSS) | (premature | (TSS to | intrinsic | ID |
| name^{a} | gene | Gene annotation | position^{b} | terminator) | terminator) | terminator?^{c} | NO |
| TBR-EF1 | EF0097 | regulatory protein pfoR | 100977 | 101073 | 97 | no | 31 |
| TBR-EF2 | EF0167 | GMP synthase | 166135 | 165960 | 176 | no | 32 |
| TBR-EF3 | EF0660 | MATE efflux family protein | 611263 | 611360 | 98 | yes | 33 |
| TBR-EF4 | EF0820 | L25 50S ribosomal protein | 784369 | 784259 | 111 | yes | 34 |
| TBR-EF5 | EF0846 | DEAD/DEAH box helicase | 804919 | 805025 | 107 | yes | 35 |
| TBR-EF6 | EF0904 | mevalonate kinase | 870961 | 870852 | 110 | yes | 36 |
| TBR-EF7 | EF1147 | CTP synthetase | 1115883 | 1115958 | 76 | yes | 37 |
| TBR-EF8 | EF1413 | msrC protein | 1392104 | 1392274 | 171 | yes | 38 |
| TBR-EF9 | EF1492 | V-type ATPase subunit F | 1447297 | 1447439 | 143 | yes | 39 |
| TBR-EF10 | EF2645 | transcriptional regulator | 2558275 | 2558190 | 86 | no | 40 |
| TBR-EF11 | EF2720 | ABC transporter | 2632870 | 2632727 | 144 | yes | 41 |
| TBR-EF12 | EF2858 | threonyl-tRNA synthetase | 2741757 | 2741525 | 233 | yes | 42 |
| TBR-EF13 | EF3022 | sodium: dicarboxylate symporter | 2900136 | 2900253 | 118 | yes | 43 |
| TBR-EF14 | EF3157 | glycosyl hydrolase | 3028244 | 3028341 | 98 | no | 44 |

^{a}Serial number of novel Termination-Based-Regulators (TBRs) discovered in this study in E. faecalis
^{b}TSSs were inferred from transcriptome-wide sequencing of RNA 5′ ends³² (Methods).
^{c}Indicates whether the term-seq position was preceded by a stem-loop-polyU signature, indicative of intrinsic terminators.

In L. monocytogenes, many of the elements found were previously annotated as small RNAs or as potential cis-acting regulatory 5′UTRs^{32,45}; the present data supports that they are indeed cis-acting regulators (FIGS. 2F-G; Table 4). The conditional-termination-based elements detected regulate genes of diverse functions, including those involved in virulence. For example, deletion of the conditional-termination regulator of lmo0559, called rli31 (FIG. 2F), led to an attenuated virulence phenotype of L. monocytogenes in mouse and butterfly larvae infection models as well as in studies of murine macrophage infection⁴⁶. Deletion of rli31 also led to decreased lysozyme resistance of L. monocytogenes in blood⁴⁷. In addition, it was found that Mpl (lmo0203), a zinc metalloprotease important for the cell-to-cell spread of L. monocytogenes⁴⁸, is preceded by a premature-terminator (FIG. 2G). Interestingly, it was previously shown that Mpl expression is increased in blood cultures, and its 5′UTR was suggested to contain a cis-acting regulator responsive to growth in blood conditions⁴⁵.

These results imply that the new termination-based regulatory elements detected may control multiple physiological- and pathogenicity-relevant processes. Moreover, the present results, in which 44 potential new regulators in merely three genomes were mapped, strongly support previous predictions that numerous yet-unknown termination-based regulators are encoded in bacteria²³, and demonstrate the ability of term-seq to rapidly map such regulators across bacterial genomes.

### Example 3

### Genome-Wide Metabolite Screening in Physiological Conditions

While the data reveal multiple cases of new candidate regulators, they do not expose the metabolites to which these regulators respond. Discovery of the metabolite that controls the activity of given candidate regulator, either by direct binding (riboswitch) or indirectly (via attenuation or anti-termination proteins), is a major challenge in the field, usually involving in-vitro structural probing of the regulator in the presence of various candidate metabolites and/or the construction of reporter assays to monitor the activity of the regulator^{3,49,50}.

The present inventors developed a term-seq based strategy that allows rapid evaluation of multiple possible metabolites across all candidate regulators simultaneously in physiological, in-vivo conditions. It was reasoned that the presence of the metabolite should alter the open/closed state of the regulator, and that this state can be quantitatively measured as the ratio between the full-length RNA and the short, prematurely terminated form (FIG. 3A). As term-seq directly provides a readthrough measure (quantification of short/long transcript ratios) for every expressed regulator in the genome, it enables low-cost, parallel analysis of in-vivo regulator activities following the application of any metabolite of interest.

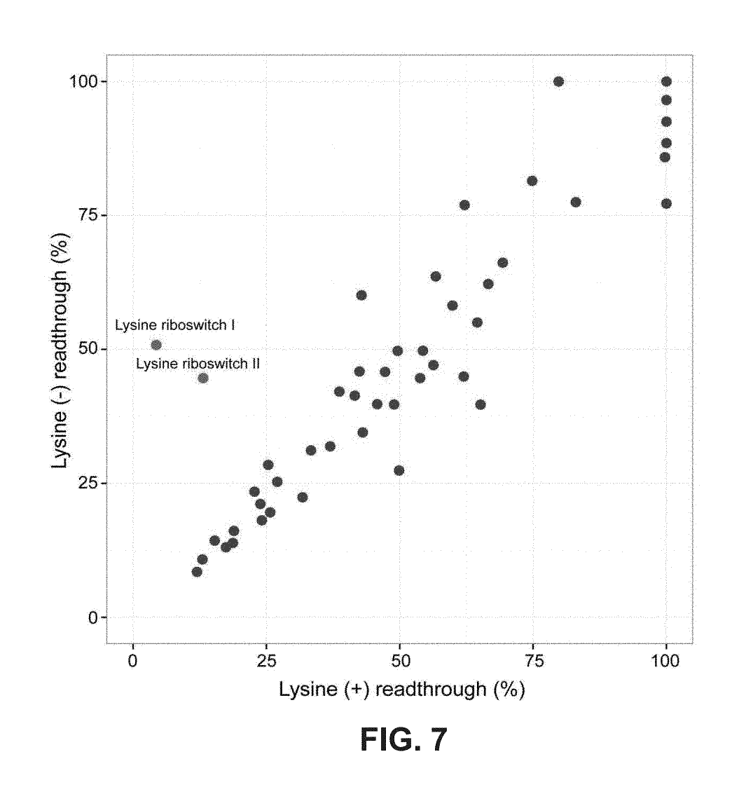

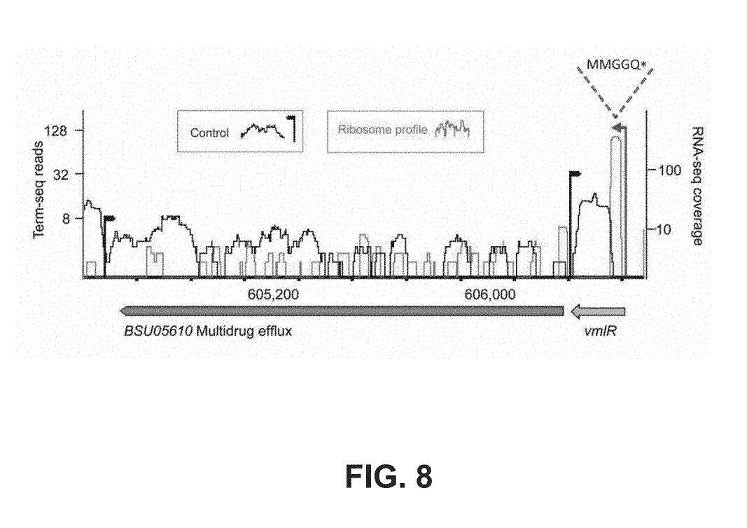

To validate this approach, B. subtilis was grown in a defined medium with or without the amino acid lysine. Out of the 82 regulators mapped in B. subtilis, it was found that only the two known lysine riboswitches showed a significant increase in readthrough level as a result of lysine depletion (FIGS. 3B-C; FIG. 7). Moreover, depletion of a different amino acid from the medium (methionine, FIGS. 3B-C) did not increase the open/closed ratio of the lysine riboswitches, pointing to their high specificity in sensing the presence of lysine.

### Example 4

### Antibiotic-Controlled Conditional Termination Regulates Antibiotics Resistance Genes

As inducible antibiotic resistance mechanisms pose a major medical challenge, the present inventors set out to use this approach to screen for regulators that respond to the presence of antibiotics. More than two decades ago it was reported that the rRNA methylase gene ermK, which confers resistance to macrolide-lincosamide-streptogramin B antibiotics, is controlled by conditional premature termination that is alleviated when the antibiotic is introduced⁵¹. Since then, sporadic reports described similar regulation in a few additional antibiotics resistance genes, but this mode of regulation was considered rare^{39,41}. To understand the extent of this phenomenon the present inventors searched for antibiotic-responsive regulators by applying term-seq to B. subtilis, L. monocytogenes and E. faecalis following short exposure to sublethal doses of seven different antibiotics (Table 6). Strikingly, six of the regulators that were reported above (Tables 3-5) showed a strong, antibiotic-dependent response, characterized by significant readthrough into the downstream gene in the presence of the antibiotic (FIGS. 4A-G and FIGS. 10A-F).

TABLE 6
Sublethal concentrations of antibiotics used in this study

| Antibiotic (μg/ml) | B. subtilis | L. monocytogenes | E. faecalis |
|---|---|---|---|
| Lincomycin | 0.5 | 0.25 | 2 |
| Chloramphenicol | 0.2 | 0.4 | 0.4 |
| Erythromycin | 0.015625 | 0.03125 | 0.5 |
| Kanamycin | 0.5 | 0.5 | 16 |
| Ciprofloxacin | 0.0625 | 2 | 1 |
| Ampicillin | 0.0625 | 1 | 2 |
| Bacitracin | 0.125 | 8 | 1 |

Two antibiotic-responsive, termination-based regulatory elements were observed in each of the three bacteria studied. In B. subtilis, vmlR⁴¹ and the recently described bmrB³⁹ regulators were identified, which were shown to regulate the expression of antibiotic resistance genes through an unknown or ribosome-mediated mechanism, respectively (FIGS. 4A-B). In L. monocytogenes, however, two new regulators that were previously annotated as conserved Listeria small RNAs of unknown function, rli53 and rli59, that were hypothesized to possibly act in cis⁴⁵ were discovered. It was found that these two sRNAs function as antibiotic responsive cis-regulatory elements that control the expression of the genes lmo0919 and lmo1652, both encoding ABC transporter genes of unknown function (FIGS. 4C-D). Whereas the alteration in the open/closed state of lmo0919 by rli53 was highly specific to lincomycin, rli59 was more permissive and responded to several different translation inhibiting antibiotics (FIGS. 4C and 4G).

In E. faecalis, it was previously shown that expression of the msrC macrolide-resistance efflux pump increases in response to erythromycin exposure⁵². The present data suggests that the msrC response results from a coordinated activity of its promoter and a novel termination-based regulator, which, upon exposure to the antibiotic, act in concert to increase both the transcription initiation rate and readthrough into the gene (FIGS. 4E, 4G). Interestingly, a similar promoter-termination synchronized activity was detected for the B. subtilis vmlR gene (FIG. 4B). Finally, an additional lincomycin-specific termination-based regulator that controls the expression of yet another ABC transporter of unknown function, EF2720, in E. faecalis (FIG. 4F) was found.

The presence of antibiotic-responsive, termination-based regulatory elements upstream of specific genes in L. monocytogenes and E. faecalis mark the regulated genes as possible novel antibiotic resistance genes. The present inventors further characterized the antibiotic-based regulation of lmo0919, an ABC transporter of unknown function in L. monocytogenes. This gene was previously suggested to be involved in antibiotic resistance based on its distant homology to the staphylococcal Vga gene and its heterologous activity in staphylococcal hosts⁵³, but its function in L. monocytogenes remained unknown. Remarkably, it was found that deletion of lmo0919 rendered L. monocytogenes 4-fold more sensitive to the antibiotics lincomycin, but did not reduce the MIC of other antibiotic classes (Table 7).

TABLE 7

| | L. mono- | | ΔAnti- | | uORF |
|---|---|---|---|---|---|
| | cytogenes | | anti- | ΔAnti- | ATG > |
| MIC (μg/ml) | Wt | Δlmo0919 | terminator | terminator | ACG |
| Lincomycin | 1 | 0.25 | 8 | 0.25 | 0.25 |
| Erythromycin | 0.03125 | 0.0625 | 0.03125 | 0.0625 | 0.0625 |
| Chloram- | 2 | 2 | 2 | 2 | 2 |
| phenicol | | | | | |

The protein encoded by lmo0919 therefore confers lincomycin-specific antibiotic resistance, consistent with the specific activation of its 5′UTR regulator by lincomycin but not by erythromycin or chloramphenicol (FIGS. 4D and 4G).

### Example 5

### The Mechanism of Antibiotics-Mediated Conditional Termination in lmo0919

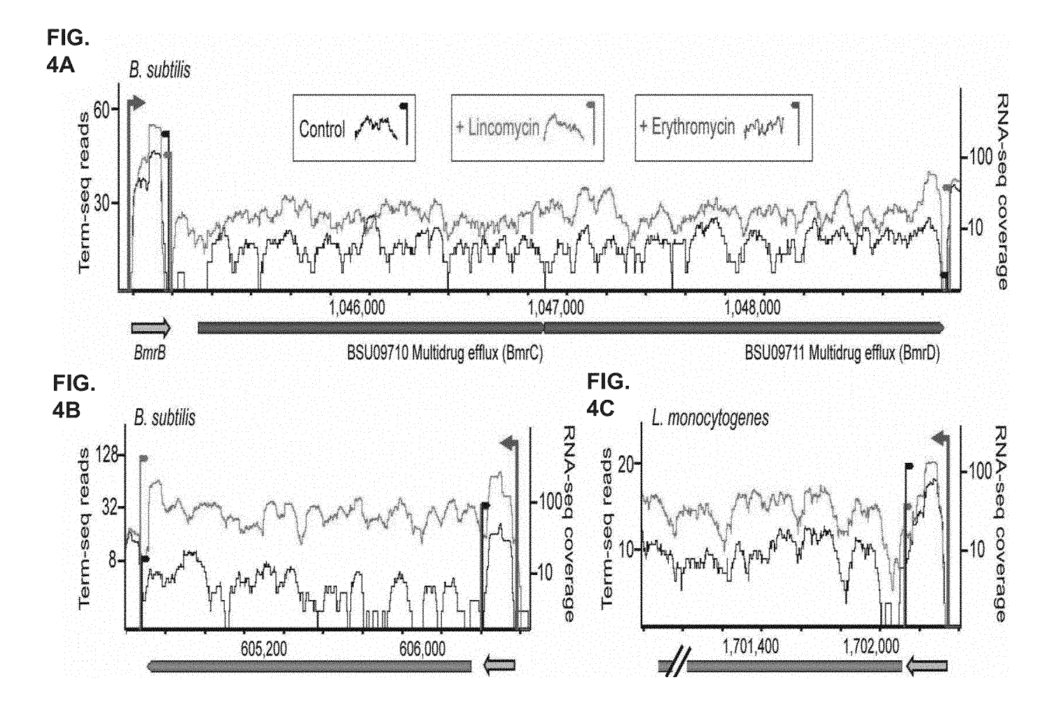

Inspection of the regulatory 5′ UTR sequence of lmo0919 revealed a potential two-stem, terminator/antiterminator structure (FIG. 5A). Such structures are common in riboswitches and attenuators, and can adopt two alternative conformations, one that generates a transcriptional terminator (FIG. 5A, left) and another in which the anti-terminator promotes transcriptional read-through by interfering with terminator formation (FIG. 5A, right). To enquire whether this mode of regulation occurs in the case of lmo0919, mutations were performed in either the first or the second arm of the first stem, disrupting the putative anti-anti-terminator or the anti-terminator, respectively (FIG. 5B). Consistent with the model, deletion of 8 nucleotides from the anti-terminator kept the regulator in a constitutively “closed” state even in the presence of lincomycin antibiotic, rendering the bacteria sensitive to otherwise sublethal dose of lincomycin (FIGS. 5C-E and 5G). In contrast, deletion of 8 nucleotides from the anti-anti-terminator freed the anti-terminator to interfere with the terminator structure, leading to constitutive read-through (“open” state) even in the absence of antibiotics, and resulting in increased resistance to lincomycin (FIGS. 5B and 5F). These results support a model in which the lincomycin-dependent activation of lmo0919 expression is mediated by a structural interplay of terminator/anti-terminator structures in the 5′UTR of this gene.

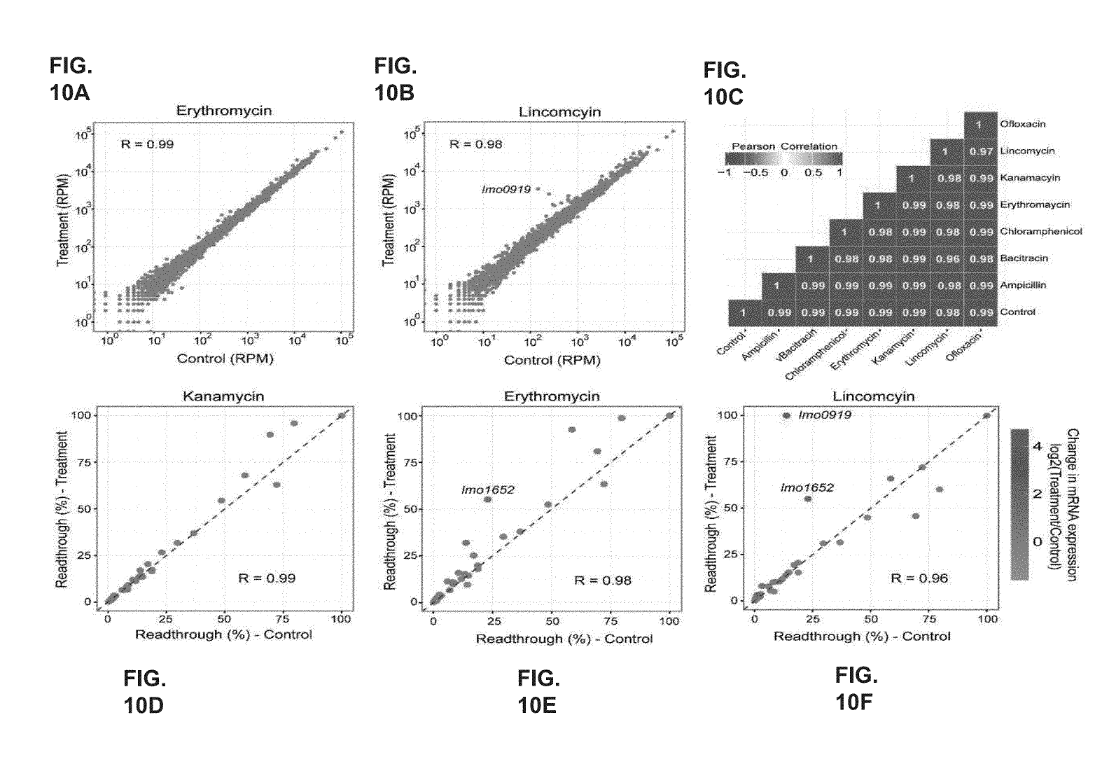

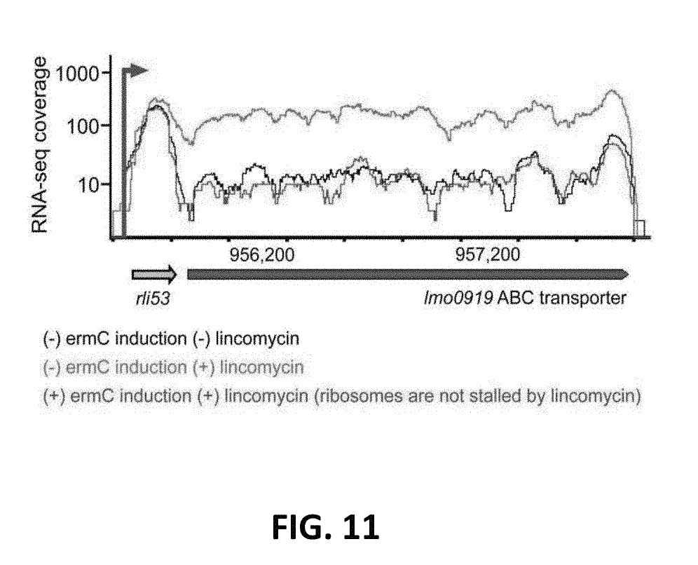

The structural alterations in the lmo0919 ribo-regulator could either be mediated by direct binding of the antibiotic to the ribo-regulator (i.e., a riboswitch), or by attenuation, where the lincomycin-inhibited ribosomes stall on a uORF in the ribo-regulator, thus shifting the ribo-regulator structure from a “closed” to an “open” state. To differentiate between direct binding and ribosome-mediated regulation, lincomycin-dependent induction of lmo0919 in L. monocytogenes expressing the ErmC 23S rRNA methyltransferase was measured. In these bacteria the ribosomes are di-methylated at position A2058 of the 23S rRNA, rendering the ribosomes resistant to lincomycin. Hence, in this experimental system, lincomycin is present in the cell but does not inhibit the ribosome. Strikingly, in ErmC-expressing bacteria the lmo0919 regulator was no longer responsive to lincomycin (FIG. 11), suggesting that this ribo-regulator depends on stalled ribosomes for its activity and does not interact directly with the antibiotic molecule.

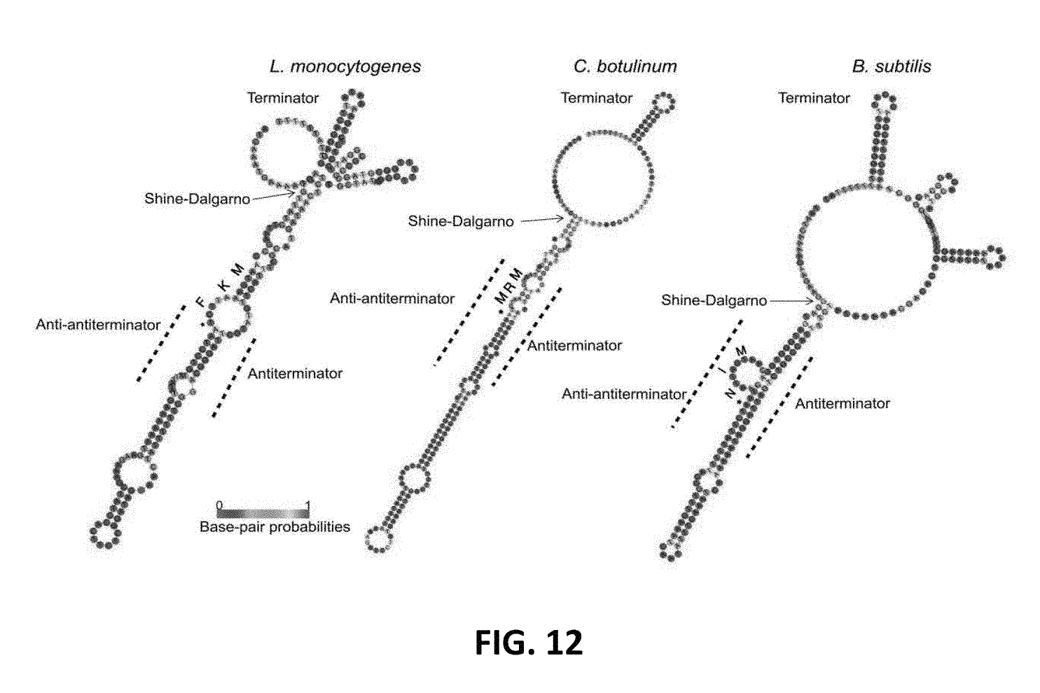

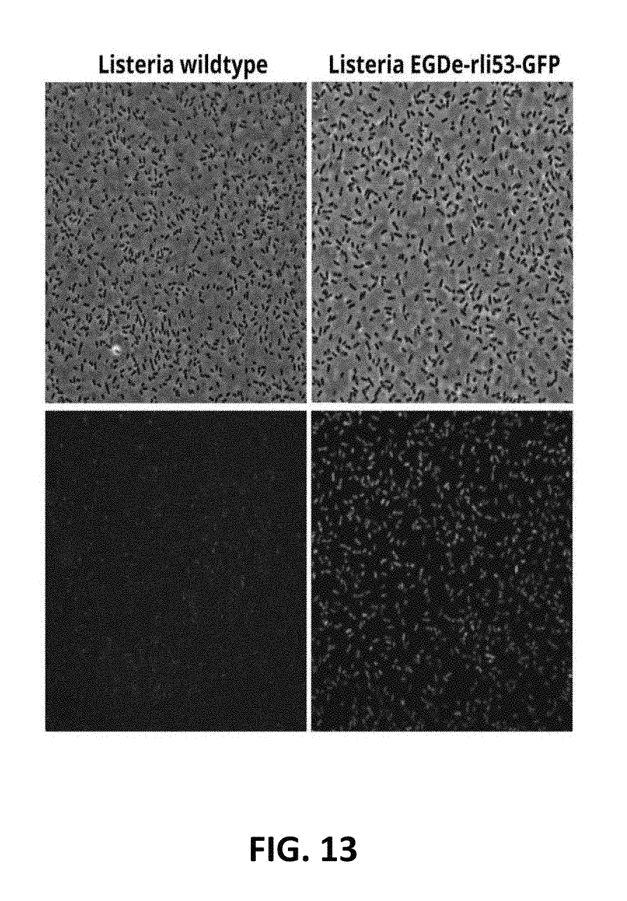

To gain a higher resolution insight into the lmo0919 ribo-regulator a comparative analysis of the 5′UTR sequences of lmo0919 homologs in various Gram positive bacteria was performed (23). While the nucleotide sequence of the ribo-regulator showed almost no conservation between species, the terminator/anti-terminator architecture was strictly conserved among all homologs (FIG. 12). Remarkably, despite the near lack of sequence conservation, all regulators contained a 3-amino-acid uORF exactly overlapping the inhibitory anti-anti-terminator sequence (FIG. 5B; FIG. 12). Although such tiny ORFs were never previously reported to be involved in transcriptional attenuation, the strong positional conservation of the uORF in the ribo-regulator led to the hypothesis that its translation forms the basis for the attenuation-based regulation. Indeed, a GFP fusion assay showed that this uORF is translated in L. monocytogenes in-vivo (FIG. 13).

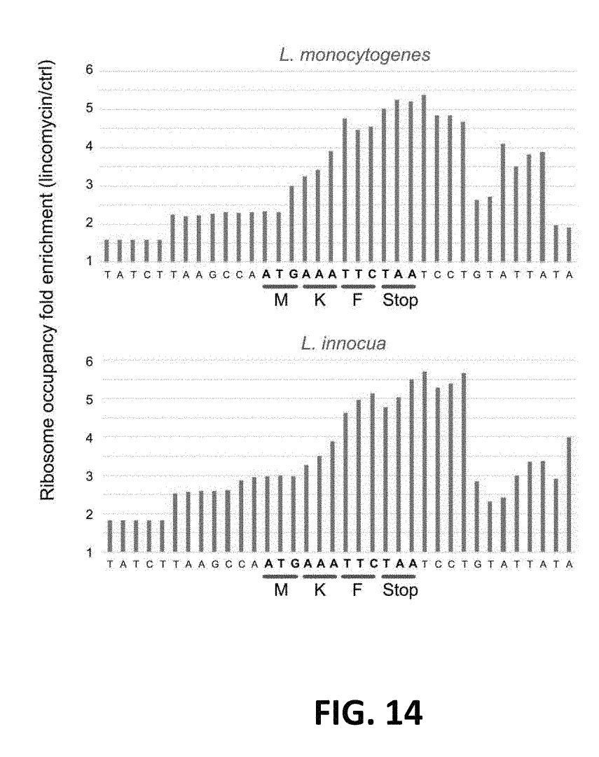

To characterize the effects of lincomycin on the interaction between the ribo-regulator and the ribosome levels of ribosome occupancy over the ribo-regulator in control and lincomycin-treated bacteria were measured using ribosome profiling (Ribo-seq) (23, 42). Strikingly, an ˜5 fold increase in ribosome occupancy over the 3aa uORFs in both L. monocytogenes and L. innocua was measured following brief exposure to lincomycin (FIG. 14) (23). These results show that lincomycin-inhibited ribosomes specifically stall at the 3aa uORF that overlaps the anti-anti-terminator sequence.

Collectively, these results point to an attenuation-based regulatory mechanism, where the association of the ribosome with the antibiotic leads the ribosome to stall on a tiny uORF that overlaps the anti-anti-terminator, releasing the anti-terminator to interfere with terminator folding, and thus allowing read-through into the antibiotic resistance gene. Indeed, a single-base mutation that changed the ATG (Met) initiation codon of the uORF into ACG led to suppressed lincomycin dependent read-through, validating the model (FIGS. 5B, 5D, and 5H).

Notably, the lmo0919 regulator is specifically activated by lincomycin but not by erythromycin (FIGS. 4A-G), although both antibiotics induce ribosome stalling. It was previously shown that while antibiotics of the lincomycin family inhibit ribosome progression after the incorporation of 1-2 amino acids, erythromycin requires the addition of 6-8 amino acids to the nascent chain before it stalls the ribosome (43). It is therefore likely that the specificity of the lmo0919 ribo-regulator to lincomycin stems from the short size of its functional uORF.

### Example 5

### Meta-Term-Seq Reveals an Abundance of Antibiotics Regulators in the Human Microbiome

In order to examine whether termination-based regulation commonly controls antibiotic resistance in nature, the human oral microbiome was probed for such regulatory elements. The human microbiome is a complex microbial community comprised of hundreds of commensal teeth- and mouth-associated species that are frequently naturally exposed to antibiotics.

Instead of culturing and growing each of the hundreds of oral microbiota species separately, a meta-transcriptomics approach was used (denoted here meta-term-seq) in order to probe the transcriptional profile of the microbial consortium in a single experiment. For this, teeth-associated bacteria were sampled using a toothpick from three healthy individuals and were pooled in tubes containing BHI medium with and without the antibiotic lincomycin for 15 minutes. Following RNA extraction, term-seq and RNA-seq was applied on the pooled RNA, and the resulting RNA reads were mapped to the >400 reference genomes that were sequenced as part of the human oral microbiome project (54, 55) (Methods). The antibiotic-responsive meta-term-seq profiles in the 167 species that showed significant expression of at least 10% of their genes were studied (Methods).

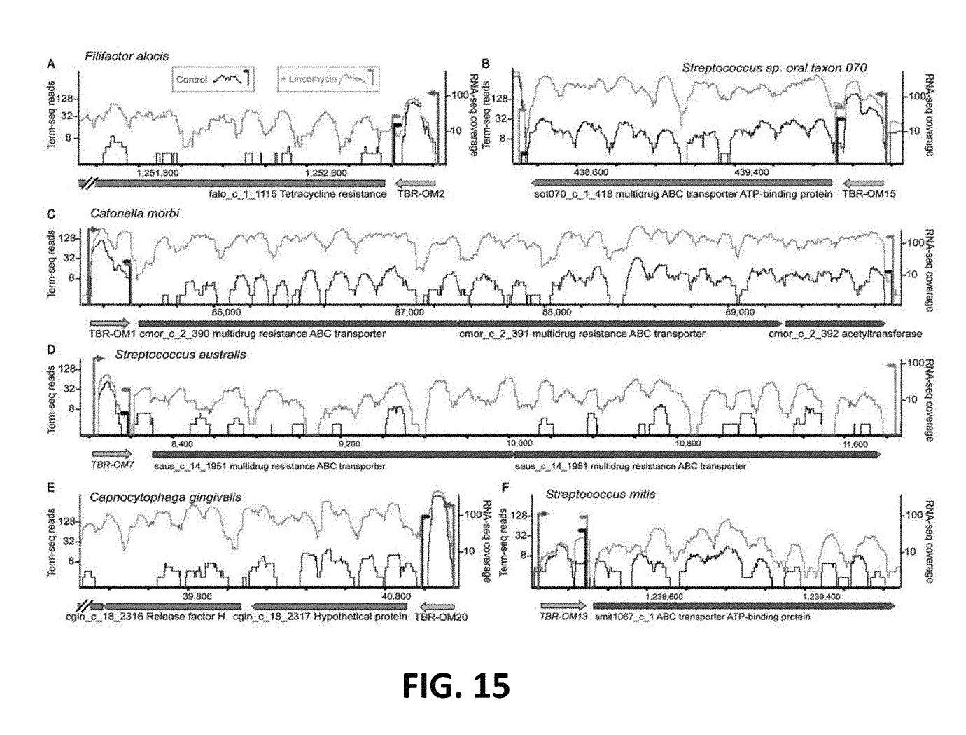

Remarkably, operons activated by alleviation of premature termination in response to lincomycin were abundantly found in members of the human oral microbiome. 21 regulatory elements were detected, overall controlling 57 genes, in which transcriptional read-through was significantly increased following the application of lincomycin (FIGS. 9A-B; FIG. 15, Table 4; Methods). Such elements were detected in 21% (13/61) of the Firmicutes present in the studied set, indicating that this mode of regulation is common in bacteria belonging to this phylum. The genes regulated by the antibiotic-responsive cis-acting RNA elements included several different classes of multidrug antibiotics exporters and efflux pumps (56, 57), rRNA methylases known to confer antibiotics resistance via modification of the ribosomal RNA (51), acetyltransferases known to directly deactivate the antibiotic via acetylation (58), genes annotated as tetracycline resistance small-GTPases that rescue antibiotic-bound ribosomes (59), and additional genes that were not described so far as conferring antibiotic resistance (FIG. 6; Table 8).

TABLE 8

| | | | | | | | SEQ |
|---|---|---|---|---|---|---|---|
| | | Genome | Locus tag(s) of | | Term-seq | Regulator | ID |
| Name | Organism | scaffold | genes in regulated operon | TSS | position | length | NO: |
| TBR-OM1 | Catonella morbi | cmor_c_2 | cmor_c_2_390-392 | 85320 | 85536 | 215 | 89 |
| TBR-OM2 | Filifactor alocis | falo_c_1 | falo_c_1_1115 | 1253255 | 1253026 | 228 | 101 |
| TBR-OM3 | Lachnospiraceae bacterium | slon_c_4 | slon_c_4_1311-12 | 55945 | 56198 | 252 | 102 |
| TBR-OM4 | Selenomonas infelix | sinfelix_c_16 | sinfelix_c_16_2172-2174 | 14588 | 14694 | 105 | 103 |
| TBR-OM5 | Streptococcus anginosus | sang_c_46 | sang_c_46_1574-1575 | 11076 | 10908 | 167 | 104 |
| TBR-OM6 | Streptococcus australis | saus_c_12 | saus_c_12_1884 | 68589 | 68677 | 87 | 105 |
| TBR-OM7 | Streptococcus australis | saus_c_14 | saus_c_14_1951-1952 | 8035 | 8177 | 141 | 106 |
| TBR-OM8 | Streptococcus cristatus | scri_c_1 | scri_c_1_263 | 253680 | 253950 | 269 | 107 |
| TBR-OM9 | Streptococcus cristatus | scri_c_1 | scri_c_1_71-76 | 68678 | 68841 | 162 | 108 |
| TBR-OM10 | Streptococcus cristatus | scri_c_3 | scri_c_3_719-720 | 69341 | 69497 | 155 | 109 |
| TBR-OM11 | Streptococcus gordonii | sgor_c_1 | sgor_c_1_1278 | 1327538 | 1327809 | 270 | 110 |
| TBR-OM12 | Streptococcus mitis | smit1067_c_1 | smit1067_c_1_323-326 | 323118 | 323432 | 313 | 111 |
| TBR-OM13 | Streptococcus mitis | smit1067_c_1 | smit1067_c_1_1236 | 1238009 | 1238278 | 268 | 112 |
| TBR-OM14 | Streptococcus sobrinus | ssob_c_5 | ssob_c_5_198 | 25210 | 25467 | 256 | 113 |
| TBR-OM15 | Streptococcus sp. oral taxon 070 | sot070_c_1 | sot070_c_1_418 | 440204 | 439934 | 269 | 114 |
| TBR-OM16 | Streptococcus sp. oral taxon 056 | sot056_c_1 | sot056_c_1_405 | 369338 | 369608 | 269 | 115 |
| TBR-OM17 | Streptococcus sp. oral taxon 056 | sot066_c_1 | sot066_c_1_129-130 | 147370 | 147124 | 245 | 116 |
| TBR-OM18 | Actinomyces odontolyticus | NCBIAAYI_c_1 | NCBIAAYI_c_1_1375-1377 | 1645100 | 1644922 | 177 | 117 |
| TBR-OM19 | Porphyromonas sp. oral taxon 279 | pot279_c_7 | pot279_c_7_978 | 52481 | 52682 | 200 | 118 |
| TBR-OM20 | Capnocytophaga gingivalis | cgin_c_18 | cgin_c_18_2306-2317 | 41133 | 41002 | 130 | 119 |
| TBR-OM21 | Capnocytophaga sputigena | NCBIABZV_c_8 | NCBIABZV_c_8_1392-1399 | 97216 | 97396 | 179 | 120 |

These results highlight meta-term-seq as a new method for probing gene regulation in microbial consortia, and reveal a common control mechanism for antibiotic-resistance genes in human-associated bacteria.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

1. Winkler, W., Nahvi, A. & Breaker, R. R. Thiamine derivatives bind messenger RNAs directly to regulate bacterial gene expression. Nature 419, 952-956 (2002).
2. Mandal, M., Boese, B., Barrick, J. E., Winkler, W. C. & Breaker, R. R. Riboswitches control fundamental biochemical pathways in Bacillus subtilis and other bacteria. Cell 113, 577-586 (2003).
3. Sudarsan, N., Wickiser, J. K., Nakamura, S., Ebert, M. S. & Breaker, R. R. An mRNA structure in bacteria that controls gene expression by binding lysine. Genes Dev. 17, 2688-2697 (2003).
4. Green, N. J., Grundy, F. J. & Henkin, T. M. The T box mechanism: tRNA as a regulatory molecule. FEBS Lett. 584, 318-24 (2010).
5. Yanofsky, C. Attenuation in the control of expression of bacterial operons. Nature 289, 751-758 (1981).
6. Santangelo, T. J. & Artsimovitch, I. Termination and antitermination: RNA polymerase runs a stop sign. Nat. Rev. Microbiol. 9, 319-29 (2011).
7. Dann, C. E. et al., Structure and mechanism of a metal-sensing regulatory RNA. Cell 130, 878-92 (2007).
8. Furukawa, K. et al., Bacterial riboswitches cooperatively bind ni(2+) or co(2+) ions and control expression of heavy metal transporters. Mol. Cell 57, 1088-98 (2015).
9. Winkler, W. C., Cohen-Chalamish, S. & Breaker, R. R. An mRNA structure that controls gene expression by binding FMN. Proc. Natl. Acad. Sci. U.S.A. 99, 15908-13 (2002).
10. Winkler, W. C., Nahvi, A., Sudarsan, N., Barrick, J. E. & Breaker, R. R. An mRNA structure that controls gene expression by binding S-adenosylmethionine. Nat Struct Biol. 10, 701-707 (2003).
11. Irnov, I. & Winkler, W. C. A regulatory RNA required for antitermination of biofilm and capsular polysaccharide operons in Bacillales. Mol. Microbiol. 76, 559-75 (2010).
12. Sudarsan, N. et al., Riboswitches in eubacteria sense the second messenger cyclic di-GMP. Science 321, 411-3 (2008).
13. Loh, E. et al., A trans-acting riboswitch controls expression of the virulence regulator PrfA in Listeria monocytogenes. Cell 139, 770-9 (2009).
14. Mellin, J. R. et al., Sequestration of a two-component response regulator by a riboswitch-regulated noncoding RNA. Science (80-.). 345, 940-943 (2014).
15. Paige, J. S., Nguyen-Duc, T., Song, W. & Jaffrey, S. R. Fluorescence Imaging of Cellular Metabolites with RNA. Science (80-.). 335, 1194-1194 (2012).
16. Fowler, C. C., Brown, E. D. & Li, Y. Using a riboswitch sensor to examine coenzyme B12 metabolism and transport in E. coli. Chem. Biol. 17, 756-765 (2010).
17. Isaacs, F. J., Dwyer, D. J. & Collins, J. J. RNA synthetic biology. Nat. Biotechnol. 24, 545-554 (2006).
18. Benenson, Y. Synthetic biology with RNA: Progress report. Curr. Opin. Chem. Biol. 16, 278-284 (2012).
19. Blount, K. F. & Breaker, R. R. Riboswitches as antibacterial drug targets. Nat. Biotechnol. 24, 1558-1564 (2006).
20. Blount, K. F., Wang, J. X., Lim, J., Sudarsan, N. & Breaker, R. R. Antibacterial lysine analogs that target lysine riboswitches. Nat. Chem. Biol. 3, 44-49 (2007).
21. Lee, E. R., Blount, K. F. & Breaker, R. R. Roseoflavin is a natural antibacterial compound that binds to FMN riboswitches and regulates gene expression. RNA Biol. 6, 187-94 (2009).
22. Mulhbacher, J. et al., Novel Riboswitch Ligand Analogs as Selective Inhibitors of Guanine-Related Metabolic Pathways. PLoS Pathog. 6, e1000865 (2010).
23. Breaker, R. R. Prospects for riboswitch discovery and analysis. Mol. Cell 43, 867-79 (2011).
24. Yao, Z. et al., A computational pipeline for high-throughput discovery of cis-regulatory noncoding RNA in prokaryotes. PLoS Comput. Biol. 3, e126 (2007).
25. Weinberg, Z. et al., Comparative genomics reveals 104 candidate structured RNAs from bacteria, archaea, and their metagenomes. Genome Biol. 11, R31 (2010).
26. Barrick, J. E. Predicting riboswitch regulation on a genomic scale. Methods Mol. Biol. 540, 1-13 (2009).
27. Sorek, R. & Cossart, P. Prokaryotic transcriptomics: a new view on regulation, physiology and pathogenicity. Nat. Rev. Genet. 11, 9-16 (2010).
28. Güell, M., Yus, E., Lluch-Senar, M. & Serrano, L. Bacterial transcriptomics: what is beyond the RNA horiz-ome? Nat. Rev. Microbiol. 9, 658-69 (2011).
29. Winkler, W. C. Riboswitches and the role of noncoding RNAs in bacterial metabolic control. Curr. Opin. Chem. Biol. 9, 594-602 (2005).
30. Peters, J. M., Vangeloff, A. D. & Landick, R. Bacterial transcription terminators: the RNA 3′-end chronicles. J. Mol. Biol. 412, 793-813 (2011).
31. Wurtzel, O. et al., A single-base resolution map of an archaeal transcriptome. Genome Res. 20, 133-41 (2010).
32. Wurtzel, O. et al., Comparative transcriptomics of pathogenic and non-pathogenic Listeria species. Mol. Syst. Biol. 8, 583 (2012).
33. Lorenz, R. et al., ViennaRNA Package 2.0. Algorithms Mol. Biol. 6, 26 (2011).
34. Crooks, G. E., Hon, G., Chandonia, J.-M. & Brenner, S. E. WebLogo: a sequence logo generator. Genome Res. 14, 1188-90 (2004).
35. Sarsero, J. P., Merino, E. & Yanofsky, C. A Bacillus subtilis operon containing genes of unknown function senses tRNATrp charging and regulates expression of the genes of tryptophan biosynthesis. Proc. Natl. Acad. Sci. U.S.A. 97, 2656-2661(2000).
36. Holmberg, C. & Rutberg, L. An inverted repeat preceding the Bacillus subtilis glpD gene is a conditional terminator of transcription. Mol. Microbiol. 6, 2931-2938 (1992).
37. Turner, R. J., Lu, Y. & Switzer, R. L. Regulation of the Bacillus subtilis pyrimidine biosynthetic (pyr) gene cluster by an autogenous transcriptional attenuation mechanism. J. Bacteriol. 176, 3708-3722 (1994).
38. Fujita, Y. Carbon catabolite control of the metabolic network in Bacillus subtilis. Biosci. Biotechnol. Biochem. 73, 245-259 (2009).
39. Reilman, E., Mars, R. a T., van Dijl, J. M. & Denham, E. L. The multidrug ABC transporter BmrC/BmrD of Bacillus subtilis is regulated via a ribosome-mediated transcriptional attenuation mechanism. Nucleic Acids Res. 42, 11393-11407 (2014).
40. Naville, M. & Gautheret, D. Premature terminator analysis sheds light on a hidden world of bacterial transcriptional attenuation. Genome Biol. 11, R97 (2010).
41. Ohki, R., Tateno, K., Takizawa, T., Aiso, T. & Murata, M. Transcriptional termination control of a novel ABC transporter gene involved in antibiotic resistance in Bacillus subtilis. J. Bacteriol. 187, 5946-5954 (2005).
42. Nawrocki, E. P. et al., Rfam 12.0: updates to the RNA families database. Nucleic Acids Res. 43, D130-D137 (2014).
43. De Noordhout, C. M. et al., The global burden of listeriosis: a systematic review and meta-analysis. Lancet Infect. Dis. 14, 1073-1082 (2014).
44. Sava, I. G., Heikens, E. & Huebner, J. Pathogenesis and immunity in enterococcal infections. Clin. Microbiol. Infect. 16, 533-540 (2010).
45. Toledo-Arana, A. et al., The Listeria transcriptional landscape from saprophytism to virulence. Nature 459, 950-6 (2009).
46. Mraheil, M. a. et al., The intracellular sRNA transcriptome of Listeria monocytogenes during growth in macrophages. Nucleic Acids Res. 39, 4235-4248 (2011).
47. Burke, T. P. et al., Listeria monocytogenes Is Resistant to Lysozyme through the Regulation, Not the Acquisition, of Cell Wall-Modifying Enzymes. J. Bacteriol. 196, 3756-3767 (2014).
48. Marquis, H., Goldfine, H. & Portnoy, D. a. Proteolytic pathways of activation and degradation of a bacterial phospholipase C during intracellular infection by Listeria monocytogenes. J. Cell Biol. 137, 1381-1392 (1997).
49. Regulski, E. E. & Breaker, R. R. In-line probing analysis of riboswitches. Methods Mol. Biol. 419, 53-67 (2008).
50. Dambach, M. et al., The Ubiquitous yybP-ykoY Riboswitch Is a Manganese-Responsive Regulatory Element. Mol. Cell 57, 1099-109 (2015).
51. Kwak, J. H., Choi, E. C. & Weisblum, B. Transcriptional attenuation control of ermK, a macrolide-lincosamide-streptogramin B resistance determinant from Bacillus licheniformis. J. Bacteriol. 173, 4725-4735 (1991).
52. Aakra, Å. et al., Transcriptional Response of Enterococcus faecalis V583 to Erythromycin. Antimicrob. Agents Chemother. 49, 2246-2259 (2005).
53. Chesneau, O., Ligeret, H., Hosan-Aghaie, N., Morvan, A. & Dassa, E. Molecular analysis of resistance to streptogramin A compounds conferred by the Vga proteins of staphylococci. Antimicrob. Agents Chemother. 49, 973-80 (2005).
54. Kelemen, G. H., Zaiacain, M., Culebras, E. & Seno, E. T. Transcriptional attenuation tylosin-resistance gene tIrA. Mol. Micrcbiology 14, 833-842 (1994).
55. Douthwaite, S., Crain, P. F., Liu, M. & Poehlsgaard, J. The tylosin-resistance methyltransferase RlmAII (T1rB) modifies the N−1 position of 23 S rRNA nucleotide G748. J. Mol. Biol. 337, 1073-1077 (2004).
56. Li, G.-W., Oh, E. & Weissman, J. S. The anti-Shine-Dalgarno sequence drives translational pausing and codon choice in bacteria. Nature 484, 538-41 (2012).
57. Kim, P. B., Nelson, J. W. & Breaker, R. R. An ancient riboswitch class in bacteria regulates purine biosynthesis and one-carbon metabolism. Mol. Cell 57, 317-28 (2015).
58. Breaker, R. R. & Joyce, G. F. The expanding view of RNA and DNA function. Chem. Biol. 21, 1059-1065 (2014).
59. Arnaud, M., Chastanet, A. & Débarbouillé, M. New vector for efficient allelic replacement in naturally nontransformable, low-GC-content, gram-positive bacteria. Appl. Environ. Microbiol. 70, 6887-6891 (2004).
60. De Hoon, M. J. L., Makita, Y., Nakai, K. & Miyano, S. Prediction of transcriptional terminators in Bacillus subtilis and related species. PLoS Comput. Biol. 1, e25 (2005).
61. Burge, S. W. et al., Rfam 11.0: 10 years of RNA families. Nucleic Acids Res. 41, D226-32 (2013).

1. An isolated polynucleotide comprising a nucleic acid sequence as set forth in SEQ ID NOs: 1-44 operatively linked to a heterologous nucleic acid sequence.

2. The isolated polynucleotide of claim 1, wherein said heterologous nucleic acid sequence encodes a polypeptide.

3. The isolated polynucleotide of claim 2, wherein said polypeptide is a human polypeptide.

4. The isolated polynucleotide of claim 2, wherein said polypeptide is a reporter polypeptide comprising a detectable moiety.

5. The isolated polynucleotide of claim 4, wherein said detectable moiety is a fluorescent moiety or a phosphorescent moiety.

6. The isolated polynucleotide of claim 1, being operatively linked to a promoter.

7. The isolated polynucleotide of claim 6, wherein said promoter is a bacterial promoter.

8. An isolated RNA comprising a nucleic acid sequence as set forth in SEQ ID NOs: 45-88, or a DNA encoding same, wherein the RNA or DNA is no longer than 450 nucleotides.

9. An RNA aptamer comprising a nucleic acid sequence as set forth in SEQ ID NOs: 45-88 operatively linked to a signal generating moiety.

10. The RNA aptamer of claim 9, wherein said nucleic acid sequence is as set forth in SEQ ID NOs: 67, 71, 82 and 85.

11. The RNA aptamer of claim 9, wherein said signal generating moiety is encoded by a heterologous nucleic acid sequence.

12. The RNA aptamer of claim 11, wherein said heterologous nucleic acid sequence encodes a polypeptide.

13. The RNA aptamer of claim 9, wherein said signal generating moiety comprises a fluorescent moiety or a phosphorescent moiety.

14. A bacteria genetically modified to express the isolated polynucleotide of claim 1.

15. A cell which comprises the aptamer of claim 9.

16. A bacteria genetically modified to express the isolated polynucleotide of claim 1, wherein said isolated polynucleotide comprises a nucleic acid sequence as set forth in SEQ ID NOs: 23, 27, 38 and 41 and said heterologous nucleic acid sequences encodes a reporter polypeptide.

17. The bacteria of claim 16, wherein said reporter polypeptide comprises a fluorescent moiety or a phosphorescent moiety.

18. A method of detecting an antibiotic in a sample comprising:
(a) culturing a L. monocytogenes or E. faecalis bacteria in a medium comprising said sample;
(b) analyzing the number of full length RNA transcripts transcribed from the bacterial gene selected from the group consisting of lmo0919, lmo1652, EF1413 and EF2720 and prematurely terminated RNA transcripts transcribed from said bacterial gene; and
(c) comparing the ratio of prematurely terminated RNA transcripts transcribed from said bacterial gene: full length RNA transcripts transcribed from said bacterial gene in the presence of the sample to the ratio of prematurely terminated RNA transcripts transcribed from said bacterial gene: full length RNA transcripts transcribed from said bacterial gene in the absence of the sample, wherein a statistically significant change in said ratio is indicative that the sample comprises an antibiotic.

19. A method of detecting an antibiotic in a sample comprising:
(a) culturing the bacteria of claim 16 in a medium comprising said sample; and
(b) measuring a level of expression of said reporter polypeptide, wherein a change in said level of expression of said reporter polypeptide as compared to the level of said reporter polypeptide measured when the bacteria of claim 16 are cultured in a medium devoid of an antibiotic, is indicative that the sample comprises an antibiotic.

20. A method of detecting an antibiotic in a sample comprising:
(a) contacting the aptamer of claim 10 with said sample;
(b) measuring the signal generated by said signal generating moiety, wherein a level of said signal above a predetermined threshold is indicative that the sample comprises an antibiotic.

21. The method of claim 19, wherein said sample is a body fluid.

22. The method of claim 21, wherein said body fluid is selected from the group consisting of saliva, blood, serum, milk and urine.

23. The method of claim 19, wherein said sample is an environmental sample.

24. A method of determining whether an agent is a transcription terminator comprising:
(a) culturing the bacteria of claim 14 in a medium comprising said agent; and
(b) measuring the level of expression of said reporter polypeptide, wherein a change in said level of expression of said reporter polypeptide as compared to the level of said reporter polypeptide measured when the bacteria of claim 14 are cultured in a medium devoid of said agent is indicative that the agent is a transcription terminator.

25. A method of identifying if an agent is an antibiotic, the method comprising:
determining whether the agent is a transcription terminator according to claim 24; and
testing an effect of said transcription terminator on vitality of bacterial cells, wherein a level of vitality of bacterial cells below a predetermined amount is indicative that the agent is an antibiotic.

26. A method of controlling expression of a gene product comprising contacting a bacteria with a ligand of a ligand responsive element, wherein the bacteria comprises a nucleic acid sequence encoding the gene product, the nucleic acid sequence being operatively linked to:
(i) said ligand responsive element, wherein said ligand responsive element comprises a sequence as set forth in SEQ ID NOs: 1-44; and
(ii) a promoter, thereby controlling expression of the gene product, thereby controlling expression of the gene product.

27. The method of claim 26, further comprising removing the ligand from the bacteria.

28. The method of claim 27, wherein said removing is effected by contacting the bacteria with an RNA aptamer comprising a nucleic acid sequence as set forth in SEQ ID NOs: 45-88.

29. A method of determining a transcription termination site in bacterial DNA:
(a) ligating a first adaptor to the 3′ end of RNA transcripts of a bacterial RNA sample to generate elongated RNA transcripts;
(b) fragmenting said elongated RNA transcripts:
(c) combining the elongated RNA transcripts with a reverse transcriptase and an oligonucleotide that hybridizes to said adaptor under conditions that allow synthesis of cDNA from said elongated RNA transcripts;
(d) ligating a second adaptor to the 3′ end of said cDNA to generate elongated cDNA transcripts;
(e) amplifying said elongated cDNA transcripts using primers that hybridize to the sequence of said first adaptor and the sequence of said second adaptor to generate amplified DNA; and
(f) sequencing said amplified DNA, thereby determining the transcription termination site in bacterial DNA.

30. A method of determining a transcription termination site in bacterial DNA:
(a) ligating a first adaptor to the 3′ end of RNA transcripts of a bacterial RNA sample to generate elongated RNA transcripts;
(b) fragmenting said elongated RNA transcripts to generate fragmented RNA transcripts:
(c) ligating a second adaptor to the 5′ end of said fragmented RNA transcripts to generate elongated fragmented RNA transcripts;
(d) combining the elongated fragmented RNA transcripts with a reverse transcriptase and an oligonucleotide that hybridizes to said first adaptor under conditions that allow synthesis of cDNA from said elongated fragmented RNA transcripts;
(e) amplifying said cDNA transcripts using primers that hybridize to the sequence of said first adaptor and the sequence of said second adaptor to generate amplified DNA; and
(f) sequencing said amplified DNA, thereby determining the transcription termination site in bacterial DNA.

31. The method of claim 29, wherein said transcription termination site is a premature transcription termination site.

32. The method of claim 29, wherein said transcription termination site is a mature transcription termination site.

33. A method of determining whether a ligand can control premature transcription termination of a bacterial gene comprising:
(a) culturing bacteria in a medium comprising the ligand;
(b) analyzing the number of full length RNA transcripts transcribed from the bacterial gene and the number of prematurely terminated RNA transcripts transcribed from the bacterial gene according to the method of claim 29; and
(c) comparing the ratio of prematurely terminated RNA transcripts transcribed from the bacterial gene: full length RNA transcripts transcribed from the bacterial gene in the presence of the ligand to the ratio of prematurely terminated RNA transcripts transcribed from the bacterial gene: full length RNA transcripts transcribed from the bacterial gene in the absence of the ligand, wherein a statistically significant change in said ratio is indicative that the ligand can control premature transcription termination of the bacterial gene.

34. The method of claim 33, wherein said ligand is selected from the group consisting of an antibiotic, a metabolite, a vitamin, an amino acid, a metal ion and a peptide.

35. The method of claim 33, wherein said ligand controls the premature termination via a riboswitch or attenuator.

36. The method of claim 33, wherein said bacteria are comprised in a heterogeneous population of bacteria.

37. The method of claim 33, wherein said bacteria are comprised in a microbiome.

38. A method of determining whether a ligand can control premature transcription termination of a bacterial gene comprising:
(a) culturing bacteria in a medium comprising the ligand;
(b) analyzing the number of full length RNA transcripts transcribed from the bacterial gene and the number of prematurely terminated RNA transcripts transcribed from the bacterial gene according to the method of claim 30; and
(c) comparing the ratio of prematurely terminated RNA transcripts transcribed from the bacterial gene: full length RNA transcripts transcribed from the bacterial gene in the presence of the ligand to the ratio of prematurely terminated RNA transcripts transcribed from the bacterial gene: full length RNA transcripts transcribed from the bacterial gene in the absence of the ligand, wherein a statistically significant change in said ratio is indicative that the ligand can control premature transcription termination of the bacterial gene.
